SEQUENCE LISTING (33 sequences)

SEQ ID NO: 1 - protein, 1086 aa, Plasmodium falciparum
MFRIFFTLLIIILIKKTSAIDLIEGIFYEKNEIDKLTFSLDHRVRDNLKTDLILNNNGENDYAYLNKYVYTILNRDSTEKIKTFFSHNKDMKSCDYFISKEYNSSDKTNQICYKKTFCGVVIPNSEEIKTNKITNDKLYCAHFNSTHIIIYYISQPLLLEPHVVYEETFFEKGKNDQINCQGMYISLRSVHVHTHNAILQQETLTYIKNLCDGKNNCKFDFDSIKYENKSLTHYLFFINIQYQCISPLNLQENEMCDVYNDDTHKATCKYGFNKIELLKNVCEENYRCTQDICSVNQFCDGENETCTCKTSLLPSAKNNCEYNDLCTVLNCPENSTCEQIGNGKKAECKCENGKYYHNNKCYTKNDLELAIKIEPHKKEKFYKNNLYQGKALKPEYIFMQCENGFSIEVINAYVSCYRVSFNLNKLKYVTESLKKMCDGKTKCAYGNTIDPIDDLNHHNICNNFNTIFKYDYLCVFNNQNITSDKNSHLHSNIPSLYNSSILPDINKSKFHLISRNSRTNQYPHNNISMLEIQNEISSHNSNQFSTDPHTNSNNINNMNIKKVEIFRSRFSSKLQCQGGKINIDKAILKGGEGCNDLLLTNSLKSYCNDLSECDIGLIYHFDTYCINDQYLFVSYSCSNLCNKCHNNSTCYGNRFNYDCFCDNPYISKYGNKLCERPNDCESVLCSQNQVCQILPNDKLICQCEEGYKNVKGKCVPDNKCDLSCPSNKVCVIENGKQTCKCSERFVLENGVCICANDYKMEDGINCIAKNKCKRKEYENICTNPNEMCAYNEETDIVKCECKEHYYRSSRGECILNDYCKDINCKENEECSIVNFKPECVCKENLKKNNKGECIYENSCLINEGNCPKDSKCIYREYKPHECVCNKQGHVAVNGKCVLEDKCVHNKKCSENSICVNVMNKEPICVCTYNYYKKDGVCLIQNPCLKDNGGCSRNSECTFKYSKINCTCKENYKNKDDSCVPNTNEYDESFTFQYNDDASIILGACGMIEFSYIYNQIIWKINNSKESYVFYYDYPTAGNIEVQIKNEIFHTIIYLKKKIGNSVIYDDFQVDHQTCIYENVFYYSNQN

SEQ ID NO: 2 - protein, 185 aa, Plasmodium falciparum
PDNKCDLSCPSNKVCVIENGKQTCKCSERFVLENGVCICANDYKMEDGINCIAKNKCKRKEYENICTNPNEMCAYNEETDIVKCECKEHYYRSSRGECILNDYCKDINCKENEECSIVNFKPECVCKENLKKNNKGECIYENSCLINEGNCPKDSKCIYREYKPHECVCNKQGHVAVNGKCVLED

SEQ ID NO: 3 - protein, 81 aa, Plasmodium falciparum
DYCKDINCKENEECSIVNFKPECVCKENLKKNNKGECIYENSCLINEGNCPKDSKCIYREYKPHECVCNKQGHVAVNGKCV

SEQ ID NO: 4 - protein, 107 aa, Plasmodium falciparum
PNTNEYDESFTFQYNDDASIILGACGMIEFSYIYNQIIWKIQNSKESYVFYYDYPTAGNIEVQIKNEIFHTIIYLKKKIGNSVIYDDFQVDHQTCIYENVFYYSNQN

SEQ ID NO: 5 - protein, 297 aa, synthetic construct
PDNKCDLSCPSNKVCVIENGKQTCKCSERFVLENGVCICANDYKMEDGINCIAKNKCKRKEYENICTNPNEMCAYNEETDIVKCECKEHYYRSSRGECILNDYCKDINCKENEECSIVNFKPECVCKENLKKNNKGECIYENSCLINEGNCPKDSKCIYREYKPHECVCNKQGHVAVNGKCVLEDGGGGTPNTNEYDESFTFQYNDDASIILGACGMIEFSYIYNQIIWKIQNSKESYVFYYDYPTAGNIEVQIKNEIFHTIIYLKKKIGNSVIYDDFQVDHQTCIYENVFYYSNQN

SEQ ID NO: 6 - protein, 192 aa, synthetic construct
DYCKDINCKENEECSIVNFKPECVCKENLKKNNKGECIYENSCLINEGNCPKDSKCIYREYKPHECVCNKQGHVAVNGKCVGGGTPNTNEYDESFTFQYNDDASIILGACGMIEFSYIYNQIIWKIQNSKESYVFYYDYPTAGNIEVQIKNEIFHTIIYLKKKIGNSVIYDDFQVDHQTCIYENVFYYSNQN

SEQ ID NO: 7 - protein, 334 aa, Plasmodium falciparum
DSRHVFIRTELSFIKNNVPCIRDMFFIYKRELYNICLDDLKGEEDETHIYVQKKVKDSWITLNDLFKETDLTGRPHIFAYVDVEEIIILLCEDEEFSNRKKDMTCHRFYSNDGKEYNNAEITISDYILKDKLLSSYVSLPLKIENREYFLICGVSPYKFKDDNKKDDILCMASHDKGETWGTKIVIKYDNYKLGVQYFFLRPYISKNDLSFHFYVGDNINNVKNVNFIECTHEKDLEFVCSNRDFLKDNKVLQDVSTLNDEYIVSYGNDNNFAECYIFFNNENSILIKPEKYGNTAAGCYGGTFVKIDENRALFIYSSSQGIYNIHTIYYANYE

SEQ ID NO: 8 - protein, 13 aa, synthetic construct
AHIVMVDAYKPTK

SEQ ID NO: 9 - protein, 33 aa, synthetic construct
GGGGSGGGGSAHIVMVDAYKPTKGGGGSGGGGS

SEQ ID NO: 10 - protein, 552 aa, synthetic construct
PDNKCDLSCPSNKVCVIENGKQTCKCSERFVLENGVCICANDYKMEDGINCIAKNKCKRKEYENICTNPNEMCAYNEETDIVKCECKEHYYRSSRGECILNDYCKDINCKENEECSIVNFKPECVCKENLKKNNKGECIYENSCLINEGNCPKDSKCIYREYKPHECVCNKQGHVAVNGKCVLEDGGGGSGGGGSAHIVMVDAYKPTKGGGGSGGGGSDSRHVFIRTELSFIKNNVPCIRDMFFIYKRELYNICLDDLKGEEDETHIYVQKKVKDSWITLNDLFKETDLTGRPHIFAYVDVEEIIILLCEDEEFSNRKKDMTCHRFYSNDGKEYNNAEITISDYILKDKLLSSYVSLPLKIENREYFLICGVSPYKFKDDNKKDDILCMASHDKGETWGTKIVIKYDNYKLGVQYFFLRPYISKNDLSFHFYVGDNINNVKNVNFIECTHEKDLEFVCSNRDFLKDNKVLQDVSTLNDEYIVSYGNDNNFAECYIFFNNENSILIKPEKYGNTAAGCYGGTFVKIDENRALFIYSSSQGIYNIHTIYYANYE

SEQ ID NO: 11 - protein, 448 aa, Synthetic construct
DYCKDINCKENEECSIVNFKPECVCKENLKKNNKGECIYENSCLINEGNCPKDSKCIYREYKPHECVCNKQGHVAVNGKCVGGGGSGGGGSAHIVMVDAYKPTKGGGGSGGGGSDSRHVFIRTELSFIKNNVPCIRDMFFIYKRELYNICLDDLKGEEDETHIYVQKKVKDSWITLNDLFKETDLTGRPHIFAYVDVEEIIILLCEDEEFSNRKKDMTCHRFYSNDGKEYNNAEITISDYILKDKLLSSYVSLPLKIENREYFLICGVSPYKFKDDNKKDDILCMASHDKGETWGTKIVIKYDNYKLGVQYFFLRPYISKNDLSFHFYVGDNINNVKNVNFIECTHEKDLEFVCSNRDFLKDNKVLQDVSTLNDEYIVSYGNDNNFAECYIFFNNENSILIKPEKYGNTAAGCYGGTFVKIDENRALFIYSSSQGIYNIHTIYYANYE

SEQ ID NO: 12 - protein, 580 aa, synthetic construct
MGWSCIILFLVATATGVHSPDNKCDLSCPSNKVCVIENGKQTCKCSERFVLENGVCICANDYKMEDGINCIAKNKCKRKEYENICTNPNEMCAYNEETDIVKCECKEHYYRSSRGECILNDYCKDINCKENEECSIVNFKPECVCKENLKKNNKGECIYENSCLINEGNCPKDSKCIYREYKPHECVCNKQGHVAVNGKCVLEDGGGGSGGGGSAHIVMVDAYKPTKGGGGSGGGGSDSRHVFIRTELSFIKNNVPCIRDMFFIYKRELYNICLDDLKGEEDETHIYVQKKVKDSWITLNDLFKETDLTGRPHIFAYVDVEEIIILLCEDEEFSNRKKDMTCHRFYSNDGKEYNNAEITISDYILKDKLLSSYVSLPLKIENREYFLICGVSPYKFKDDNKKDDILCMASHDKGETWGTKIVIKYDNYKLGVQYFFLRPYISKNDLSFHFYVGDNINNVKNVNFIECTHEKDLEFVCSNRDFLKDNKVLQDVSTLNDEYIVSYGNDNNFAECYIFFNNENSILIKPEKYGNTAAGCYGGTFVKIDENRALFIYSSSQGIYNIHTIYYANYEGGGGSEPEA

SEQ ID NO: 13 - protein, 476 aa, synthetic construct
MGWSCIILFLVATATGVHSDYCKDINCKENEECSIVNFKPECVCKENLKKNNKGECIYENSCLINEGNCPKDSKCIYREYKPHECVCNKQGHVAVNGKCVGGGGSGGGGSAHIVMVDAYKPTKGGGGSGGGGSDSRHVFIRTELSFIKNNVPCIRDMFFIYKRELYNICLDDLKGEEDETHIYVQKKVKDSWITLNDLFKETDLTGRPHIFAYVDVEEIIILLCEDEEFSNRKKDMTCHRFYSNDGKEYNNAEITISDYILKDKLLSSYVSLPLKIENREYFLICGVSPYKFKDDNKKDDILCMASHDKGETWGTKIVIKYDNYKLGVQYFFLRPYISKNDLSFHFYVGDNINNVKNVNFIECTHEKDLEFVCSNRDFLKDNKVLQDVSTLNDEYIVSYGNDNNFAECYIFFNNENSILIKPEKYGNTAAGCYGGTFVKIDENRALFIYSSSQGIYNIHTIYYANYEGGGGSEPEA

SEQ ID NO: 14 - protein, 330 aa, synthetic construct
PDNKCDLSCPSNKVCVIENGKQTCKCSERFVLENGVCICANDYKMEDGINCIAKNKCKRKEYENICTNPNEMCAYNEETDIVKCECKEHYYRSSRGECILNDYCKDINCKENEECSIVNFKPECVCKENLKKNNKGECIYENSCLINEGNCPKDSKCIYREYKPHECVCNKQGHVAVNGKCVLEDGGGGTPNTNEYDESFTFQYNDDASIILGACGMIEFSYIYNQIIWKIQNSKESYVFYYDYPTAGNIEVQIKNEIFHTIIYLKKKIGNSVIYDDFQVDHQTCIYENVFYYSNQNGGGGSGGGGSAHIVMVDAYKPTKGGGGSGGGGS

SEQ ID NO: 15 - protein, 664 aa, synthetic construct
PDNKCDLSCPSNKVCVIENGKQTCKCSERFVLENGVCICANDYKMEDGINCIAKNKCKRKEYENICTNPNEMCAYNEETDIVKCECKEHYYRSSRGECILNDYCKDINCKENEECSIVNFKPECVCKENLKKNNKGECIYENSCLINEGNCPKDSKCIYREYKPHECVCNKQGHVAVNGKCVLEDGGGGTPNTNEYDESFTFQYNDDASIILGACGMIEFSYIYNQIIWKIQNSKESYVFYYDYPTAGNIEVQIKNEIFHTIIYLKKKIGNSVIYDDFQVDHQTCIYENVFYYSNQNGGGGSGGGGSAHIVMVDAYKPTKGGGGSGGGGSDSRHVFIRTELSFIKNNVPCIRDMFFIYKRELYNICLDDLKGEEDETHIYVQKKVKDSWITLNDLFKETDLTGRPHIFAYVDVEEIIILLCEDEEFSNRKKDMTCHRFYSNDGKEYNNAEITISDYILKDKLLSSYVSLPLKIENREYFLICGVSPYKFKDDNKKDDILCMASHDKGETWGTKIVIKYDNYKLGVQYFFLRPYISKNDLSFHFYVGDNINNVKNVNFIECTHEKDLEFVCSNRDFLKDNKVLQDVSTLNDEYIVSYGNDNNFAECYIFFNNENSILIKPEKYGNTAAGCYGGTFVKIDENRALFIYSSSQGIYNIHTIYYANYE

SEQ ID NO: 16 - protein, 694 aa, synthetic construct
MGWSCIILFLVATATGVHSGTPDNKCDLSCPSNKVCVIENGKQTCKCSERFVLENGVCICANDYKMEDGINCIAKNKCKRKEYENICTNPNEMCAYNEETDIVKCECKEHYYRSSRGECILNDYCKDINCKENEECSIVNFKPECVCKENLKKNNKGECIYENSCLINEGNCPKDSKCIYREYKPHECVCNKQGHVAVNGKCVLEDGGGGTPNTNEYDESFTFQYNDDASIILGACGMIEFSYIYNQIIWKIQNSKESYVFYYDYPTAGNIEVQIKNEIFHTIIYLKKKIGNSVIYDDFQVDHQTCIYENVFYYSNQNGGGGSGGGGSAHIVMVDAYKPTKGGGGSGGGGSDSRHVFIRTELSFIKNNVPCIRDMFFIYKRELYNICLDDLKGEEDETHIYVQKKVKDSWITLNDLFKETDLTGRPHIFAYVDVEEIIILLCEDEEFSNRKKDMTCHRFYSNDGKEYNNAEITISDYILKDKLLSSYVSLPLKIENREYFLICGVSPYKFKDDNKKDDILCMASHDKGETWGTKIVIKYDNYKLGVQYFFLRPYISKNDLSFHFYVGDNINNVKNVNFIECTHEKDLEFVCSNRDFLKDNKVLQDVSTLNDEYIVSYGNDNNFAECYIFFNNENSILIKPEKYGNTAAGCYGGTFVKIDENRALFIYSSSQGIYNIHTIYYANYEGGGGSEPEA

SEQ ID NO: 17 - protein, 225 aa, synthetic construct
DYCKDINCKENEECSIVNFKPECVCKENLKKNNKGECIYENSCLINEGNCPKDSKCIYREYKPHECVCNKQGHVAVNGKCVGGGTPNTNEYDESFTFQYNDDASIILGACGMIEFSYIYNQIIWKIQNSKESYVFYYDYPTAGNIEVQIKNEIFHTIIYLKKKIGNSVIYDDFQVDHQTCIYENVFYYSNQNGGGGSGGGGSAHIVMVDAYKPTKGGGGSGGGGS

SEQ ID NO: 18 - protein, 559 aa, synthetic construct
DYCKDINCKENEECSIVNFKPECVCKENLKKNNKGECIYENSCLINEGNCPKDSKCIYREYKPHECVCNKQGHVAVNGKCVGGGTPNTNEYDESFTFQYNDDASIILGACGMIEFSYIYNQIIWKIQNSKESYVFYYDYPTAGNIEVQIKNEIFHTIIYLKKKIGNSVIYDDFQVDHQTCIYENVFYYSNQNGGGGSGGGGSAHIVMVDAYKPTKGGGGSGGGGSDSRHVFIRTELSFIKNNVPCIRDMFFIYKRELYNICLDDLKGEEDETHIYVQKKVKDSWITLNDLFKETDLTGRPHIFAYVDVEEIIILLCEDEEFSNRKKDMTCHRFYSNDGKEYNNAEITISDYILKDKLLSSYVSLPLKIENREYFLICGVSPYKFKDDNKKDDILCMASHDKGETWGTKIVIKYDNYKLGVQYFFLRPYISKNDLSFHFYVGDNINNVKNVNFIECTHEKDLEFVCSNRDFLKDNKVLQDVSTLNDEYIVSYGNDNNFAECYIFFNNENSILIKPEKYGNTAAGCYGGTFVKIDENRALFIYSSSQGIYNIHTIYYANYE

SEQ ID NO: 19 - protein, 587 aa, synthetic construct
MGWSCIILFLVATATGVHSDYCKDINCKENEECSIVNFKPECVCKENLKKNNKGECIYENSCLINEGNCPKDSKCIYREYKPHECVCNKQGHVAVNGKCVGGGTPNTNEYDESFTFQYNDDASIILGACGMIEFSYIYNQIIWKIQNSKESYVFYYDYPTAGNIEVQIKNEIFHTIIYLKKKIGNSVIYDDFQVDHQTCIYENVFYYSNQNGGGGSGGGGSAHIVMVDAYKPTKGGGGSGGGGSDSRHVFIRTELSFIKNNVPCIRDMFFIYKRELYNICLDDLKGEEDETHIYVQKKVKDSWITLNDLFKETDLTGRPHIFAYVDVEEIIILLCEDEEFSNRKKDMTCHRFYSNDGKEYNNAEITISDYILKDKLLSSYVSLPLKIENREYFLICGVSPYKFKDDNKKDDILCMASHDKGETWGTKIVIKYDNYKLGVQYFFLRPYISKNDLSFHFYVGDNINNVKNVNFIECTHEKDLEFVCSNRDFLKDNKVLQDVSTLNDEYIVSYGNDNNFAECYIFFNNENSILIKPEKYGNTAAGCYGGTFVKIDENRALFIYSSSQGIYNIHTIYYANYEGGGGSEPEA

SEQ ID NO: 20 - protein, 526 aa, Plasmodium falciparum
MIRIKKKLILTIIYIHLFILNRLSFENAIKKTKNQENNLTLLPIKSTEEEKDDIKNGKDIKKEIDNDKENIKTNNAKDHSTYIKSYLNTNVNDGLKYLFIPSHNSFIKKYSVFNQINDGMLLNEKNDVKNNEDYKNVDYKNVNFLQYHFKELSNYNIANSIDILQEKEGHLDFVIIPHYTFLDYYKHLSYNSIYHKSSTYGKCIAVDAFIKKINETYDKVKSKCNDIKNDLIATIKKLEHPYDINNKNDDSYRYDISEEIDDKSEETDDETEEVEDSIQDTDSNHTPSNKKKNDLMNRTFKKMMDEYNTKKKKLIKCIKNHENDFNKICMDMKNYGTNLFEQLSCYNNNFCNTNGIRYHYDEYIHKLILSVKSKNLNKDLSDMTNILQQSELLLTNLNKKMGSYIYIDTIKFIHKEMKHIFNRIEYHTKIINDKTKIIQDKIKLNIWRTFQKDELLKRILDMSNEYSLFITSDHLRQMLYNTFYSKEKHLNNIFHHLIYVLQMKFNDVPIKMEYFQTYKKNKPLTQ

SEQ ID NO: 21 - protein, 526 aa, Plasmodium falciparum
MIRIKKKLILTIIYIHLFILNRLSFENAIKKTKNQENNLTLLPIKSTEEEKDDIKNGKDIKKEIDNDKENIKTNNAKDHSTYIKSYLNTNVNDGLKYLFIPSHNSFIKKYSVFNQINDGMLLNEKNDVKNNEDYKNVDYKNVNFLQYHFKELSNYNIANSIDILQEKEGHLDFVIIPHYTFLDYYKHLSYNSIYHKSSTYGKYIAVDAFIKKINETYDKVKSKCNDIKNDLIATIKKLEHPYDINNKNDDSYRYDISEEIDDKSEETDDETEEVEDSIQDTDSNHTPSNKKKNDLMNRTFKKMMDEYNTKKKKLIKCIKNHENDFNKICMDMKNYGTNLFEQLSCYNNNFCNTNGIRYHYDEYIHKLILSVKSKNLNKDLSDMTNILQQSELLLTNLNKKMGSYIYIDTIKFIHKEMKHIFNRIEYHTKIINDKTKIIQDKIKLNIWRTFQKDELLKRILDMSNEYSLFITSDHLRQMLYNTFYSKEKHLNNIFHHLIYVLQMKFNDVPIKMEYFQTYKKNKPLTQ

SEQ ID NO: 22 - protein, 387 aa, Plasmodium falciparum
KNVNFLQYHFKELSNYNIANSIDILQEKEGHLDFVIIPHYTFLDYYKHLSYNSIYHKSSTYGKCIAVDAFIKKINETYDKVKSKCNDIKNDLIATIKKLEHPYDINNKNDDSYRYDISEEIDDKSEETDDETEEVEDSIQDTDSNHTPSNKKKNDLMNRTFKKMMDEYNTKKKKLIKCIKNHENDFNKICMDMKNYGTNLFEQLSCYNNNFCNTNGIRYHYDEYIHKLILSVKSKNLNKDLSDMTNILQQSELLLTNLNKKMGSYIYIDTIKFIHKEMKHIFNRIEYHTKIINDKTKIIQDKIKLNIWRTFQKDELLKRILDMSNEYSLFITSDHLRQMLYNTFYSKEKHLNNIFHHLIYVLQMKFNDVPIKMEYFQTYKKNKPLTQ

SEQ ID NO: 23 - protein, 387 aa, Plasmodium falciparum
KNVNFLQYHFKELSNYNIANSIDILQEKEGHLDFVIIPHYTFLDYYKHLSYNSIYHKSSTYGKYIAVDAFIKKINETYDKVKSKCNDIKNDLIATIKKLEHPYDINNKNDDSYRYDISEEIDDKSEETDDETEEVEDSIQDTDSNHTPSNKKKNDLMNRTFKKMMDEYNTKKKKLIKCIKNHENDFNKICMDMKNYGTNLFEQLSCYNNNFCNTNGIRYHYDEYIHKLILSVKSKNLNKDLSDMTNILQQSELLLTNLNKKMGSYIYIDTIKFIHKEMKHIFNRIEYHTKIINDKTKIIQDKIKLNIWRTFQKDELLKRILDMSNEYSLFITSDHLRQMLYNTFYSKEKHLNNIFHHLIYVLQMKFNDVPIKMEYFQTYKKNKPLTQ

SEQ ID NO: 24 - protein, 367 aa, Plasmodium falciparum
SIDILQEKEGHLDFVIIPHYTFLDYYKHLSYNSIYHKSSTYGKCIAVDAFIKKINETYDKVKSKCNDIKNDLIATIKKLEHPYDINNKNDDSYRYDISEEIDDKSEETDDETEEVEDSIQDTDSNHTPSNKKKNDLMNRTFKKMMDEYNTKKKKLIKCIKNHENDFNKICMDMKNYGTNLFEQLSCYNNNFCNTNGIRYHYDEYIHKLILSVKSKNLNKDLSDMTNILQQSELLLTNLNKKMGSYIYIDTIKFIHKEMKHIFNRIEYHTKIINDKTKIIQDKIKLNIWRTFQKDELLKRILDMSNEYSLFITSDHLRQMLYNTFYSKEKHLNNIFHHLIYVLQMKFNDVPIKMEYFQTYKKNKPLTQ

SEQ ID NO: 25 - protein, 367 aa, Plasmodium falciparum
SIDILQEKEGHLDFVIIPHYTFLDYYKHLSYNSIYHKSSTYGKYIAVDAFIKKINETYDKVKSKCNDIKNDLIATIKKLEHPYDINNKNDDSYRYDISEEIDDKSEETDDETEEVEDSIQDTDSNHTPSNKKKNDLMNRTFKKMMDEYNTKKKKLIKCIKNHENDFNKICMDMKNYGTNLFEQLSCYNNNFCNTNGIRYHYDEYIHKLILSVKSKNLNKDLSDMTNILQQSELLLTNLNKKMGSYIYIDTIKFIHKEMKHIFNRIEYHTKIINDKTKIIQDKIKLNIWRTFQKDELLKRILDMSNEYSLFITSDHLRQMLYNTFYSKEKHLNNIFHHLIYVLQMKFNDVPIKMEYFQTYKKNKPLTQ

SEQ ID NO: 26 - protein, 338 aa, synthetic construct
KNVNFLQYHFKELSNYNIANSIDILQEKEGHLDFVIIPHYTFLDYYKHLSYNSIYHKSSTYGKCIAVDAFIKKINETYDKVKSKCNDIKNDLIATIKKLEHPYDINNKNRTFKKMMDEYNTKKKKLIKCIKNHENDFNKICMDMKNYGTNLFEQLSCYNNNFCNTNGIRYHYDEYIHKLILSVKSKNLNKDLSDMTNILQQSELLLTNLNKKMGSYIYIDTIKFIHKEMKHIFNRIEYHTKIINDKTKIIQDKIKLNIWRTFQKDELLKRILDMSNEYSLFITSDHLRQMLYNTFYSKEKHLNNIFHHLIYVLQMKFNDVPIKMEYFQTYKKNKPLTQ

SEQ ID NO: 27 - protein, 338 aa, synthetic construct
KNVNFLQYHFKELSNYNIANSIDILQEKEGHLDFVIIPHYTFLDYYKHLSYNSIYHKSSTYGKYIAVDAFIKKINETYDKVKSKCNDIKNDLIATIKKLEHPYDINNKNRTFKKMMDEYNTKKKKLIKCIKNHENDFNKICMDMKNYGTNLFEQLSCYNNNFCNTNGIRYHYDEYIHKLILSVKSKNLNKDLSDMTNILQQSELLLTNLNKKMGSYIYIDTIKFIHKEMKHIFNRIEYHTKIINDKTKIIQDKIKLNIWRTFQKDELLKRILDMSNEYSLFITSDHLRQMLYNTFYSKEKHLNNIFHHLIYVLQMKFNDVPIKMEYFQTYKKNKPLTQ

SEQ ID NO: 28 - protein, 318 aa, synthetic construct
SIDILQEKEGHLDFVIIPHYTFLDYYKHLSYNSIYHKSSTYGKCIAVDAFIKKINETYDKVKSKCNDIKNDLIATIKKLEHPYDINNKNRTFKKMMDEYNTKKKKLIKCIKNHENDFNKICMDMKNYGTNLFEQLSCYNNNFCNTNGIRYHYDEYIHKLILSVKSKNLNKDLSDMTNILQQSELLLTNLNKKMGSYIYIDTIKFIHKEMKHIFNRIEYHTKIINDKTKIIQDKIKLNIWRTFQKDELLKRILDMSNEYSLFITSDHLRQMLYNTFYSKEKHLNNIFHHLIYVLQMKFNDVPIKMEYFQTYKKNKPLTQ

SEQ ID NO: 29 - protein, 318 aa, synthetic construct
SIDILQEKEGHLDFVIIPHYTFLDYYKHLSYNSIYHKSSTYGKYIAVDAFIKKINETYDKVKSKCNDIKNDLIATIKKLEHPYDINNKNRTFKKMMDEYNTKKKKLIKCIKNHENDFNKICMDMKNYGTNLFEQLSCYNNNFCNTNGIRYHYDEYIHKLILSVKSKNLNKDLSDMTNILQQSELLLTNLNKKMGSYIYIDTIKFIHKEMKHIFNRIEYHTKIINDKTKIIQDKIKLNIWRTFQKDELLKRILDMSNEYSLFITSDHLRQMLYNTFYSKEKHLNNIFHHLIYVLQMKFNDVPIKMEYFQTYKKNKPLTQ

SEQ ID NO: 30 - protein, 501 aa, synthetic construct
ENAIKKTKNQENNLALLPIKSTEEEKDDIKNGKDIKKEIDNDKENIKTNNAKDHSTYIKSYLNTNVNDGLKYLFIPSHNSFIKKYSVFNQINDGMLLNEKNDVKNNEDYKNVDYKNVNFLQYHFKELSNYNIANSIDILQEKEGHLDFVIIPHYTFLDYYKHLSYNSIYHKSSTYGKCIAVDAFIKKINEAYDKVKSKCNDIKNDLIATIKKLEHPYDINNKNDDSYRYDISEEIDDKSEETDDETEEVEDSIQDTDSNHAPSNKKKNDLMNRAFKKMMDEYNTKKKKLIKCIKNHENDFNKICMDMKNYGTNLFEQLSCYNNNFCNTNGIRYHYDEYIHKLILSVKSKNLNKDLSDMTNILQQSELLLTNLNKKMGSYIYIDTIKFIHKEMKHIFNRIEYHTKIINDKTKIIQDKIKLNIWRTFQKDELLKRILDMSNEYSLFITSDHLRQMLYNTFYSKEKHLNNIFHHLIYVLQMKFNDVPIKMEYFQTYKKNKPLTQ

SEQ ID NO: 31 - protein, 523 aa, synthetic construct
MKLCILLAVVAFVGLSLGENAIKKTKNQENNLALLPIKSTEEEKDDIKNGKDIKKEIDNDKENIKTNNAKDHSTYIKSYLNTNVNDGLKYLFIPSHNSFIKKYSVFNQINDGMLLNEKNDVKNNEDYKNVDYKNVNFLQYHFKELSNYNIANSIDILQEKEGHLDFVIIPHYTFLDYYKHLSYNSIYHKSSTYGKCIAVDAFIKKINEAYDKVKSKCNDIKNDLIATIKKLEHPYDINNKNDDSYRYDISEEIDDKSEETDDETEEVEDSIQDTDSNHAPSNKKKNDLMNRAFKKMMDEYNTKKKKLIKCIKNHENDFNKICMDMKNYGTNLFEQLSCYNNNFCNTNGIRYHYDEYIHKLILSVKSKNLNKDLSDMTNILQQSELLLTNLNKKMGSYIYIDTIKFIHKEMKHIFNRIEYHTKIINDKTKIIQDKIKLNIWRTFQKDELLKRILDMSNEYSLFITSDHLRQMLYNTFYSKEKHLNNIFHHLIYVLQMKFNDVPIKMEYFQTYKKNKPLTQEPEA

SEQ ID NO: 32 - protein, 528 aa, synthetic construct
ENAIKKTKNQENQLTLLPIKSTEEEKDDIKNGKDIKKEIDNDKENIKTNNAKDHSTYIKSYLNTNVNDGLKYLFIPSHNSFIKKYSVFNQINDGMLLNEKNDVKNNEDYKNVDYKNVNFLQYHFKELSNYNIANSIDILQEKEGHLDFVIIPHYTFLDYYKHLSYNSIYHKSSTYGKCIAVDAFIKKIQETYDKVKSKCNDIKNDLIATIKKLEHPYDINNKNDDSYRYDISEEIDDKSEETDDETEEVEDSIQDTDSNHTPSNKKKNDLMNRTFKKMMDEYNTKKKKLIKCIKNHENDFNKICMDMKNYGTNLFEQLSCYNNNFCNTNGIRYHYDEYIHKLILSVKSKNLNKDLSDMTNILQQSELLLTNLNKKMGSYIYIDTIKFIHKEMKHIFNRIEYHTKIINDKTKIIQDKIKLNIWRTFQKDELLKRILDMSNEYSLFITSDHLRQMLYNTFYSKEKHLNNIFHHLIYVLQMKFNDVPIKMEYFQTYKKNKPLTQGSASGLNDIFEAQKIEWHEWSHPQFEK

SEQ ID NO: 33 - protein, 342 aa, synthetic construct
KNVNFLQYHFKELSNYNIANSIDILQEKEGHLDFVIIPHYTFLDYYKHLSYNSIYHKSSTYGKCIAVDAFIKKINEAYDKVKSKCNDIKNDLIATIKKLEHPYDINNKNRAFKKMMDEYNTKKKKLIKCIKNHENDFNKICMDMKNYGTNLFEQLSCYNNNFCNTNGIRYHYDEYIHKLILSVKSKNLNKDLSDMTNILQQSELLLTNLNKKMGSYIYIDTIKFIHKEMKHIFNRIEYHTKIINDKTKIIQDKIKLNIWRTFQKDELLKRILDMSNEYSLFITSDHLRQMLYNTFYSKEKHLNNIFHHLIYVLQMKFNDVPIKMEYFQTYKKNKPLTQEPEA

(19) United States
(12) Patent Application Publication
(10) Pub. No.: US 2026/0224681 A1
(43) Pub. Date: Aug. 6, 2026

(54) VACCINE

(71) Applicant: OXFORD UNIVERSITY INNOVATION LIMITED, Oxford (GB)

(72) Inventors: Simon J. DRAPER, Oxford (Oxfordshire) (GB); Barnabas G. WILLIAMS, Oxford (Oxfordshire) (GB); Lloyd D W KING, Oxford (Oxfordshire) (GB)

(21) Appl. No.: 19/150,604

(22) PCT Filed: Jan. 26, 2024
(86) PCT No.: PCT/GB2024/050210

§ 371 Date: Jul. 24, 2025

(30) Foreign Application Priority Data
Jan. 27, 2023 (GB) 2301244.6

(51) Int. Cl. A61K 39/015; A61K 39/00; A61P 33/06; C07K 14/445; C07K 16/20; C12N 15/62; C12N 15/86

(52) U.S. Cl. CPC A61K 39/015; A61P 33/06; C07K 14/445; C07K 16/205; C12N 15/62; C12N 15/86; A61K 2039/5258; A61K 2039/53; A61K 2039/575; C07K 2319/00; C12N 2710/10334; C12N 2710/10343

(57) ABSTRACT

The present invention relates to antigens, antibodies and vaccines for treatment or prevention of malaria.

### FIELD OF THE INVENTION

The present invention relates to antigens, antibodies and vaccines for treatment or prevention of malaria.

### BACKGROUND OF THE INVENTION

Malaria places the gravest public-health burden of all parasitic diseases, leading to ~215 million human clinical cases and ~620,000 deaths annually, with the majority of deaths in children. The infection of red blood cells (RBCs) by the blood-stage form of the Plasmodium parasite is responsible for the clinical manifestations of malaria. Examples of Plasmodium parasite include the species P. falciparum, P. vivax, P. ovale and P. malariae. The most virulent parasite species, P. falciparum, is endemic in large parts of sub-Saharan Africa and Latin America. It causes the majority of malaria deaths. It can infect RBCs of all ages and is not limited to immature RBCs. P. falciparum, is therefore of particular interest and is a major target for vaccine development, as it would be highly desirable to develop a vaccine.

The only malarial vaccine approved by the WHO for widespread use is RTS,S/AS01 (Mosquirix™), which is based on the RTS,S protein. The RTS,S protein acts by blocking infection of P. falciparum in the liver. However, this vaccine has achieved only partial efficacy (~30-50% in phase II/III clinical trials). There is therefore an urgent need for a vaccine which can protect against the disease-causing blood-stage Plasmodium parasite, potentially by emulating natural immunity or by other immune mechanisms.

Previous studies have investigated the potential for antigens to induce antibodies which are effective against blood-stage malaria parasites in vitro, using the standard growth inhibitory activity (GIA) assay. One such antigen is apical membrane antigen 1 (PfAMA1).

GIA assay investigations into other protein families involved in blood-stage Plasmodium parasite invasion of RBCs have found them to be ineffective or less effective than PfAMA1.

PfAMA1 has therefore been a major focus of research on countering blood-stage malarial parasites, with numerous clinical trials. However, antibodies against PfAMA1 appear only to be effective at an extremely high concentration. In addition, PfAMA1 induces strain-specific antibodies which are not effective against genetically diverse strains of the Plasmodium parasite (A. L. Goodman, S. J. Draper, Ann. Trop. Med. Parasitol. 104, 189 (2010)). In addition, vaccine development has been hampered by the requirement for potentially reactogenic chemical adjuvants in addition to the antigen to induce sufficient antibody responses in human subjects.

Research has also been ongoing to identify other candidate malarial antigens for vaccines. The leading blood-stage target for vaccination is now a protein known as RH5, which is essential to the parasite invasion process, as it forms a critical interaction with basigin on the surface of red blood cells (WO 2012/114125). The inventors previously manufactured an insect cell expressed recombinant protein vaccine, known as RH5.1. This vaccine works by eliciting polyclonal antibodies that block parasite invasion of red blood cells and has demonstrated a reduction in parasite growth in vivo and a delay to the time of diagnosis following a human blood-stage malaria challenge in Phase I/IIa clinical trials in UK adults. This result is the first blood-stage malaria vaccine to demonstrate a reduction in parasite growth in humans and hence it is of significant interest to the malaria field to develop new vaccines that can improve upon RH5.1.

In recent years the field has identified two other essential parasite invasion proteins, known as cysteine rich protective antigen (CyRPA) and RH5-Interacting Protein (RIPR) antigen. These proteins form a heterotrimeric complex with RH5 that is known as the RH5-CyRPA-RIPR (RCR) complex. However, to date there are no clinical vaccine candidates against other (non-RH5) components of the RCR complex.

The present invention addresses one or more of the above needs by providing RIPR antigens and fusion proteins thereof which induce potent antibodies against Plasmodium infection, vectors encoding the RIPR antigen or fusion protein, and antibodies that bind specifically to the RIPR antigen, antibody-like molecules including aptamers and peptides raised against the same antigen, together with the use thereof (either alone or in combination) in the prevention or treatment of malaria.

### SUMMARY OF THE INVENTION

As described herein, the present inventors have shown for the first time that antibodies raised following immunisation with soluble RIPR fragments demonstrate better GIA than antibodies raised against full length RIPR. In addition, the inventors have shown that the immunogenicity of the RIPR fragments can be further improved by fusing the fragments to other components within the RH5-CyRPA-RIPR (RCR) complex. In particular, attempts to improve RH5.1 vaccines by co-formulating it with RIPR and CyRPA as either a mixture or RCR complex surprisingly resulted in no improvement in the parasite growth inhibitory activity (GIA). The present inventors have shown that this lack of improvement in GIA is due to the immunodominance of RIPR, as this results in a reduced antibody response to CyRPA and RH5. As demonstrated herein, the present inventors have identified that all growth inhibitory antibodies raised to full-length RIPR bind to RIPR EGF domains 5-8. In support of this, antibodies raised following immunisation with soluble RIPR EGF (5-8) demonstrate better GIA than full length RIPR and that further increases in GIA are obtained when these RIPR EGF (5-8) domains are fused to CyRPA.

Accordingly, the present invention provides a vaccine composition comprising a RH5-Interacting Protein (RIPR) antigen, wherein said antigen is an RIPR fragment comprising one or more RIPR epidermal-growth factor like (EGF) domains. Optionally, the RIPR antigen may comprise a contiguous sequence of no more than 400 amino acids from the C-terminal portion of RIPR; optionally comprising no more than 300 amino acids, no more than 200 amino acids or no more than 100 amino acids from the C-terminal portion of RIPR. Additionally (or alternatively), the RIPR antigen may comprise (or consist of): (a) an amino acid sequence having at least 80% sequence identity to SEQ ID NO: 2, (b) EGF domains 5-8 of RIPR (e.g., wherein full length RIPR corresponds to SEQ ID NO: 1), or (c) SEQ ID NO: 2. The RIPR antigen may alternatively comprise (or consist of): (a) an amino acid sequence having at least 80% sequence identity to SEQ ID NO: 3, (b) EGF domains 7-8 or RIPR (e.g., wherein full length RIPR corresponds to SEQ ID NO: 1), or (c) SEQ ID NO: 3.

The RIPR antigen of the composition may lack one or more of the following: (a) at least 500 amino acids; preferably 600 amino acids; more preferably 700 contiguous amino acids from SEQ ID NO: 1, EGF domains 1-4 of RIPR (e.g., wherein full length RIPR corresponds to SEQ ID NO: 1), and/or EGF domains 9-10 of RIPR (e.g., wherein full length RIPR corresponds to SEQ ID NO: 1).

The inventors have shown that the inclusion of a RIPR CTD may improve the potency of the RIPR antigen. Specifically the inventors have shown that the inclusion of a CTD, or portion thereof, may induce antibodies which, although themselves are non-neutralising antibodies, nevertheless provide unexpected benefits. In particular, these non-neutralising antibodies may potentiate the GIA of antibodies raised against the RIPR antigen (e.g., EGF domains 5-8 or EGF domains 7-8). Therefore, the composition of the invention may comprise a RIPR antigen comprising a RIPR C-terminal domain (CTD) or fragment thereof. For example, the RIPR antigen may be fused to a RIPR C-terminal domain (CTD) or fragment thereof. Preferably, the CTD comprises (or consists of): (a) at least 80 amino acids, at least 90 amino acids or at least 100 amino acid from SEQ ID NO: 4, (b) a sequence having at least 80% sequence identity to SEQ ID NO: 4, and/or (c) SEQ ID NO: 4.

The RIPR antigen of the invention may comprise (or consist of): (a) a sequence having at least 80% sequence identity to SEQ ID NO: 5, or SEQ ID NO: 6 (preferably SEQ ID NO: 6) or (b) SEQ ID NO: 5 or SEQ ID NO: 6; preferably SEQ ID NO: 6.

The superior properties of the RIPR antigens, and compositions thereof, are described herein. In particular, the RIPR antigen may induce: (a) antibodies that have reduced immunodominance compared to antibodies raised against full-length RIPR, (b) a higher proportion of inhibitory antibodies compared to full-length RIPR, (c) and/or antibodies that have increased growth inhibitory activity (GIA) against the blood-stage Plasmodium parasite compared to antibodies raised against full-length RIPR. For the RIPR antigen may induce antibodies that have increased growth inhibitory activity (GIA) of at least 50% against the blood-stage Plasmodium parasite; wherein optionally (a) the growth inhibitory activity (GIA) of at least 50% is against a plurality of genetic strains of the blood-stage Plasmodium parasite, and/or (b) the Plasmodium parasite is Plasmodium falciparum.

Also provided is a vaccine composition comprising a fusion protein, the fusion protein comprising the RH5-interacting Protein (RIPR) antigen as defined in any one of the preceding claims and a CyRPA antigen. The CyRPA antigen may: (a) comprise (or consist) of at least 200 amino acids, at least 250 amino acids, or at least 300 amino acids from SEQ ID NO: 7, (b) comprise (or consist) of a sequence having at least 80% sequence identity to SEQ ID NO: 7, (c) be N-terminal to or C-terminal to the RIPR antigen, (d) be fused directly to the RIPR antigen or attached to the RIPR antigen by a linker. The linker may be a peptide linker. Additionally (or alternatively) the linker may be capable of ligating to another polypeptide. An exemplary linker may comprise (or consist of) SEQ ID NO: 8 or SEQ ID NO: 9.

When the composition comprises a fusion protein, the sequence of the fusion protein may comprise (or consist of): (a) a sequence having at least 80% sequence identity to any one of SEQ ID NO: 10 to SEQ ID NO: 13, SEQ ID NO: 15, SEQ ID NO: 16, SEQ ID NO: 18 and SEQ ID NO: 19, or (b) any one of SEQ ID NO: 10 to SEQ ID NO: 13, SEQ ID NO: 15, SEQ ID NO: 16, SEQ ID NO: 18 and SEQ ID NO: 19.

Also provided is a vaccine composition comprising a virus-like particle (VLP), wherein the VLP comprises the RH5-interacting Protein (RIPR) antigen of the invention or the fusion protein of the invention.

Any of the vaccine compositions of the invention may further comprise one or more additional Plasmodium merozoite antigen. Optionally, the one or more additional Plasmodium merozoite antigen is Reticulocyte-binding protein Homologue 5 (PfRH5) antigen. Optionally, the one or more additional Plasmodium merozoite antigen is expressed as a fusion protein. Optionally, the one or more additional Plasmodium merozoite antigen is comprised in a VLP. Optionally, the one or more additional Plasmodium merozoite antigen is expressed as a single polypeptide with the RH5-interacting Protein (RIPR) antigen of the invention or the fusion protein of the invention.

Also provided is a viral vector, RNA vaccine or DNA plasmid comprising a polynucleotide sequence encoding a RH5-interacting Protein (RIPR) antigen of the invention or the fusion protein of the invention. The viral vector may be a human or simian adenovirus, or a pox virus, optionally wherein the viral vector is an AdHu5, ChAd63, ChAdOX1, ChAdOX2 or modified vaccinia Ankara (MVA) vector. The RNA vaccine or DNA plasmid may be capable of expression in an immunised mammalian cell. The DNA plasmid may be capable of expression in a heterologous protein expression system.

The viral vector, RNA vaccine or DNA plasmid may further express a Reticulocyte-binding protein Homologue 5 (PfRH5) antigen; optionally wherein (i) the PfRH5 antigen has at least 90% sequence identity to any one of SEQ ID NO: 20 to 33; and/or (ii) the RIPR antigen and the PfRH5 antigen are expressed as a fusion protein; and/or (iii) the viral vector, RNA vaccine or DNA plasmid further expresses one or more antigens selected from PfAMA1, PfCSS, PAPTRAMP, PfEBA175, PfRH1, PfRH2a, PfRH2b, PfRH4 and/or PfAARP, or a fragment thereof.

The viral vector, RNA vaccine or DNA plasmid of the invention may be combined with: (a) (i) a viral vector, RNA vaccine or DNA plasmid that expresses a Reticulocyte-binding protein Homologue 5 (PfRH5) antigen; (ii) a PfRH5 antigen or a fusion protein comprising a PfRH5 antigen; or (iii) a VLP comprising a PfRH5 antigen or PfRH5 fusion protein; and/or (b) (i) a viral vector, RNA vaccine or DNA plasmid that expresses one or more Plasmodium merozoite antigens selected from PfRH5, PfAMA1, PfCSS, PHPTRAMP, PfEBA175, PfRH1, PfRH2a, PfRH2b, PfRH4 and/or PfAARP, or a fragment thereof; (ii) said Plasmodium merozoite antigen or a fusion protein comprising said Plasmodium merozoite antigen; or (iii) a VLP comprising a Plasmodium merozoite antigen or Plasmodium merozoite antigen fusion protein.

Also provided is an antibody, or binding fragment thereof, that specifically binds to a RIPR antigen of the invention, a fusion protein of the invention or a VLP of the invention. Optionally the antibody may be: (a) a monoclonal or polyclonal antibody, and/or (b) Fab, F(ab′)2, Fv, scFv, Fd or dAb.

Also provided is a vaccine composition comprising: (a) the viral vector, and/or RNA vaccine and/or DNA plasmid of the invention, and/or (b) the antibody of the invention.

Also provided is a vaccine composition of the invention, and/or the viral vector and/or RNA vaccine and/or DNA plasmid of the invention and/or the antibody of the invention for use in a method of treating and/or preventing of malaria. Optionally, the treatment and/or prevention comprises priming a subject with a human or simian adenovirus, for example AdHu5, ChAd63, ChAdOX1 or ChAdOX2. Further optionally, the treatment and/or prevention further comprises boosting a subject with a pox virus, for example MVA.

Also provided is the use of the vaccine composition of the invention and/or the viral vector and/or RNA vaccine and/or DNA plasmid of the invention and/or the antibody of the invention in the manufacture of a medicament for the prevention and/or treatment of malaria. Optionally, the treatment and/or prevention comprises priming a subject with a human or simian adenovirus, for example AdHu5, ChAd63, ChAdOX1 or ChAdOX2. Further optionally, the treatment and/or prevention further comprises boosting a subject with a pox virus, for example MVA.

Also provided is the vaccine composition of the invention for use in immunising a subject, wherein the RIPR antigen or fusion protein results in antibodies with a growth inhibitory activity (GIA) of at least 50% against the blood-stage Plasmodium parasite; wherein optionally: (a) the PfRH5 antigen or fusion protein results in antibodies with a growth inhibitory activity (GIA) of at least 50% against a plurality of genetic strains of the blood-stage Plasmodium parasite; and/or (b) the Plasmodium parasite is Plasmodium falciparum.

### DESCRIPTION OF FIGURES

FIG. 1: A schematic depicting the structure of RIPR (A). Specifically, RIPR is a large 120 kDa complex protein containing ten epidermal-growth factor like (EGF) domains. RIPR is highly conserved with only 19 SNPs above 0.5% frequency identified in analysed populations. The crystal structure of Cysteine rich protective antigen (CyRPA) (B). CyRPA is a smaller 40 kDa protein comprising 6 “blades”. RH5, CyRPA and RIPR form a complex (RCR complex) with CyRPA acting as the central adaptor protein.

FIG. 2: Vaccine-induced rat IgG was tested in an assay of growth inhibition activity (GIA) against the 3D7 strain of Plasmodium falciparum. (A) Anti-RH5.1, CyRPA and RIPR antibodies show GIA. (B) Quantity of GIA per unit of vaccine-specific antibody demonstrates; RH5>CyRPA>RIPR specific antibodies at inhibiting parasite growth (C). Dotted line represents 50% GIA.

FIG. 3: Vaccination with the pre-formed RCR Complex or a mixture of RH5, CyRPA and RIPR (mixed in equimolar amounts) elicits antibody responses in rats that show no improvement in the GIA compared to RH5.1 alone.

FIG. 4: Immune interference is observed following co-formulation of RH5, CyRPA and RIPR. Vaccine-induced antibody titres were measured by ELISA to (A) RH5.1 (B) CyRPA and (C) RIPR. Vaccination with RCR complex or a mixture of RH5, CyRPA and RIPR shows a significant reduction in RH5 (most potent antibodies) and CyRPA (moderately potent antibodies) antibody titres but has no impact on RIPR (least potent antibodies) titres.

FIG. 5: Rabbits immunised with full length RIPR (circle and triangle) show GIA; square=unimmunised control (A). Antigen reversal” data. Depleting antibodies against RIPR EGF (7-8) led to a significant reduction in GIA (B). Antigen reversal titrations. Inventors determined that all of the neutralising epitopes on RIPR are located in EGF (5-8) as shown by the complete reversal of GIA with this protein fragment (C). GIA from immunisations with RIPR EGF (5-8) alone is equal to immunising with full length RIPR demonstrating that only EGF (5-8) is needed for a neutralising response (D). This means the immunodominant full length RIPR protein does not need to be used to obtain the benefits of targeting the RIPR protein. Although a small reduction in overall titre is observed (E), improvement in IgG quality compensates (F).

FIG. 6: Soluble RIPR EGF domains are poorly immunogenic unless conjugated to a Virus-Like Particle. Vaccine-induced anti-RIPR rat IgG titres were measured by ELISA following vaccination with RIPR, RIPR EGF domains or Hepatitis B Surface Antigen (HBsAg) virus-like particles displaying RIPR EGF domains 5-6, 7-8 or 5-8.

FIG. 7: Model of R78C (top) and R58C (below) (A) and a protein gel (B) of the two new vaccines. R78C and R58C were designed to genetically fuse the functional section of the RIPR to CyRPA to produce a fusion combination vaccine and promote dual display of these antigens when conjugated to a SpyCatcher expressing vaccine platform. The sequence of R78C and R58C are shown (C).

FIG. 8: Vaccination with an R78C+RH5.1 co-formulation results in reduced immune interference and elicits three fold more growth inhibitory activity than RH5.1.
(A) Co-formulation of R78C with RH5.1 does not impair the anti-RH5 response.
(B) Anti-RIPR titres show R78C is more immunogenic than RIPR EGF domains (7-8).
(C) R78C induces a similar level of antibody titres as CyRPA.
(D) GIA of vaccine-induced rat IgG demonstrates that a combination of R78C with RH5.1 as a mixture or complex (RCR mini) enhances GIA compared to RH5.1.

FIG. 9: R58C is immunogenic and induces GIA both alone and in-combination with RH5.1.
(A) Anti-CyRPA ELISA Titres
(B) Anti-RIPR ELISA Titres
(C) GIA of vaccine-induced rat IgG

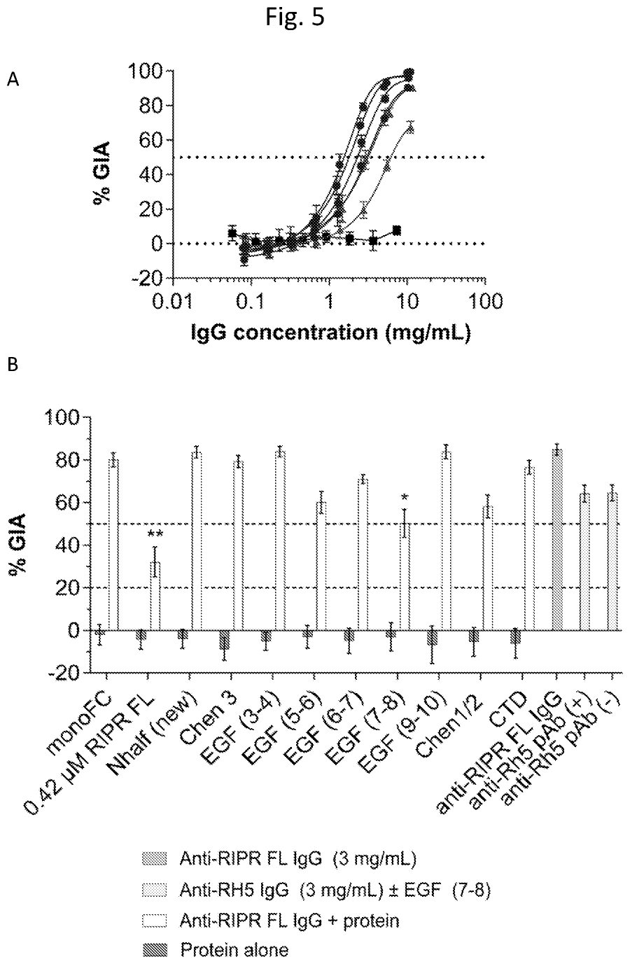

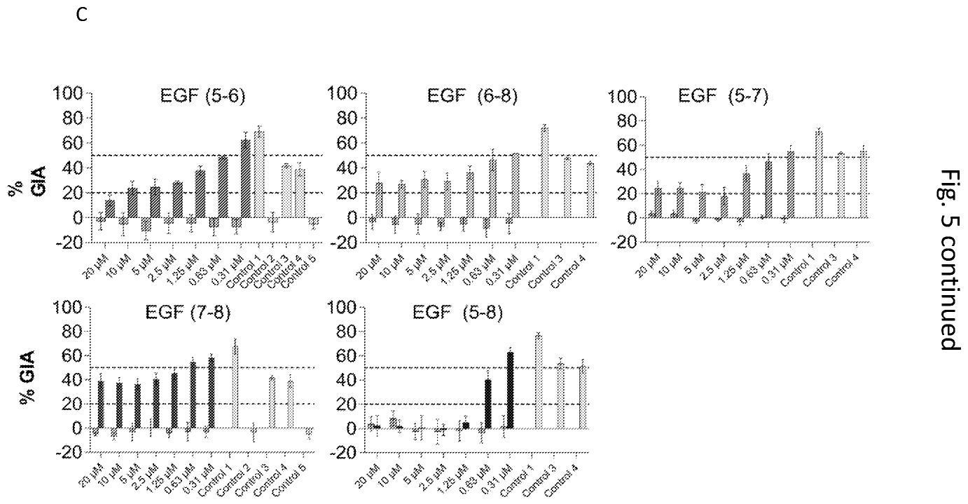

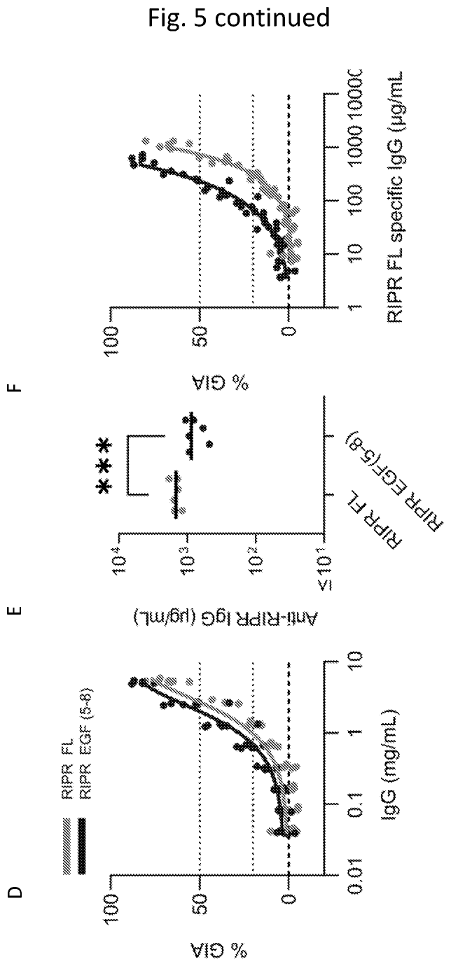

FIG. 10: The C-terminal domain (CTD) of RIPR was previously shown to have no neutralising activity (FIG. 5B). However using RIPR EGF (5-8) depleted anti-RIPR full-length polyclonal IgG (RIPR delta EGF (5-8) pAb) that has no neutralising GIA activity, the inventors discovered that the non-neutralising IgG could potentiate the activity of an anti-RIPR monoclonal antibody (mRP012) (A). The sequence of the RIPR CTD is shown in (B). The potentiating effect was due to the action of antibodies against the CTD of RIPR. A variety of protein fragments were tested to determine whether they could reverse the potentiating GIA (Nhalf, Chen3, EGF (3-4), EGF (9-10) and CTD). Only CTD reversed the GIA (arrow). Fusion of the CTD to R78C or R58C (to make R78CC or R58CC) is likely to improve the potency of the vaccines.

### DETAILED DESCRIPTION OF THE INVENTION

### Definitions

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide the skilled person with a general dictionary of many of the terms used in this disclosure. The meaning and scope of the terms should be clear; however, in the event of any latent ambiguity, definitions provided herein take precedent over any dictionary or extrinsic definition. It should be understood that this invention is not limited to the particular methodology, protocols, and reagents, etc., described herein and as such can vary.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts described herein to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

The headings provided herein are not limitations of the various aspects or embodiments of this disclosure.

As used herein, the term “capable of” when used with a verb, encompasses or means the action of the corresponding verb. For example, “capable of interacting” also means interacting, “capable of cleaving” also means cleaves, “capable of binding” also means binds and “capable of specifically targeting . . . ” also means specifically targets.

Numeric ranges are inclusive of the numbers defining the range. Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

As used herein, the articles “a” and “an” may refer to one or to more than one (e.g. to at least one) of the grammatical object of the article. Further, unless otherwise required by context, singular terms shall include pluralities and plural terms shall include the singular. In this application, the use of “or” means “and/or” unless stated otherwise. Furthermore, the use of the term “including”, as well as other forms, such as “includes” and “included”, is not limiting.

“About” may generally mean an acceptable degree of error for the quantity measured given the nature or precision of the measurements. Exemplary degrees of error are within 20 percent (%), typically, within 10%, and more typically, within 5% of a given value or range of values. Preferably, the term “about” shall be understood herein as plus or minus (±) 5%, preferably ±4%, ±3%, ±2%, ±1%, ±0.5%, ±0.1%, of the numerical value of the number with which it is being used.

The term “consisting of” refers to compositions, methods, and respective components thereof as described herein, which are exclusive of any element not recited in that description of the invention.

As used herein the term “consisting essentially of” refers to those elements required for a given invention. The term permits the presence of elements that do not materially affect the basic and novel or functional characteristic(s) of that invention (i.e. inactive or non-immunogenic ingredients).

Embodiments described herein as “comprising” one or more features may also be considered as disclosure of the corresponding embodiments “consisting of” and/or “consisting essentially of” such features.

Concentrations, amounts, volumes, percentages and other numerical values may be presented herein in a range format. It is also to be understood that such range format is used merely for convenience and brevity and should be interpreted flexibly to include not only the numerical values explicitly recited as the limits of the range but also to include all the individual numerical values or sub-ranges encompassed within that range as if each numerical value and sub-range is explicitly recited.

A “vector” or “construct” (sometimes referred to as gene delivery or gene transfer “vehicle”) refers to a macromolecule or complex of molecules comprising a polynucleotide to be delivered to a host cell, either in vitro or in vivo. A vector can be a linear or a circular molecule. A vector of the invention may be viral or non-viral. All disclosure herein in relation vectors of the invention applies equally to viral and non-viral vectors unless otherwise stated. All disclosure in relation to viral vectors of the invention applies equally and without reservation to adenovirus vectors or pox virus vectors.

As used herein, the term “plasmid”, refers to a common type of non-viral vector. A plasmid is an extra-chromosomal DNA molecule separate from the chromosomal DNA which is capable of replicating independently of the chromosomal DNA. Preferably a plasmid is circular and may be double-stranded.

The terms “nucleic acid cassette”, “nucleic acid construct”, “expression cassette” and “nucleic acid expression cassette” are used interchangeably to mean a nucleic acid molecule that is capable of directing transcription. A nucleic acid cassette includes, at the least, a promoter or a structure functionally equivalent to a promoter and a nucleic acid sequence to be transcribed. Thus, a nucleic acid cassette includes, at the least, a promoter or a structure functionally equivalent to a promoter and a nucleic acid sequence encoding a protein of interest. In the present invention, a nucleic acid cassette includes, at the least, a promoter or a structure functionally equivalent to a promoter, and a nucleic acid encoding a therapeutic protein. A nucleic acid cassette may include additional elements, such as an enhancer, and/or a transcription termination signal.

As used herein, the term “virus-like particle” (VLP) refers to a particle which resembles a virus but which does not contain viral nucleic acid and is therefore non-infectious. VLPs commonly contain one or more virus capsid or envelope proteins which are capable of self-assembly to form the VLP. VLPs have been produced from components of a wide variety of virus families (Noad and Roy (2003), Trends in Microbiology, 11:438-444; Grgacic et al., (2006), Methods, 40:60-65). Some VLPs have been approved as prophylactic vaccines, for example Engerix-B (for hepatitis B), Cervarix and Gardasil (for human papilloma viruses).

As used herein, the terms “transduced” and “modified” are used interchangeably to describe cells which have been modified to express a transgene of interest, particularly an antigen as defined herein. Typically the modification occurs through transduction of the cells.

As used herein, the terms “titre” and “yield” are used interchangeably to mean the amount of viral vector produced by a method of the invention.

Amino acids are referred to herein using the name of the amino acid, the three-letter abbreviation or the single letter abbreviation.

Unless otherwise indicated, any nucleic acid sequences are written left to right in 5′ to 3′ orientation; amino acid sequences are written left to right in amino to carboxy orientation, respectively.

As used herein, the terms “protein” and “polypeptide” are used interchangeably herein to designate a series of amino acid residues, connected to each other by peptide bonds between the alpha-amino and carboxyl groups of adjacent residues. The terms “protein”, and “polypeptide” refer to a polymer of amino acids, including modified amino acids (e.g., phosphorylated, glycated, glycosylated, etc.) and amino acid analogues, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogues of the foregoing.

As used herein, the terms “polynucleotides”, “nucleic acid” and “nucleic acid sequence” refers to any molecule, preferably a polymeric molecule, incorporating units of ribonucleic acid, deoxyribonucleic acid or an analogue thereof. The nucleic acid can be either single-stranded or double-stranded. A single-stranded nucleic acid can be one nucleic acid strand of a denatured double-stranded DNA Alternatively, it can be a single-stranded nucleic acid not derived from any double-stranded DNA. In one aspect, the nucleic acid can be DNA. In another aspect, the nucleic acid can be RNA Suitable nucleic acid molecules are DNA, including genomic DNA or cDNA. Other suitable nucleic acid molecules are RNA, including siRNA, shRNA, and antisense oligonucleotides. The terms “transgene” and “gene” are also used interchangeably and both terms encompass fragments or variants thereof encoding the target protein, specifically an antigen of the invention.

The transgenes of the present invention include nucleic acid sequences that have been removed from their naturally occurring environment, recombinant or cloned DNA isolates, and chemically synthesized analogues or analogues biologically synthesized by heterologous systems.

Minor variations in the amino acid sequences of the invention are contemplated as being encompassed by the present invention, providing that the variations in the amino acid sequence(s) maintain at least 60%, at least 70%, more preferably at least 80%, at least 85%, at least 90%, at least 95%, and most preferably at least 97% or at least 99% sequence identity to the amino acid sequence of the invention or a fragment thereof as defined anywhere herein. The term homology is used herein to mean identity. As such, the sequence of a variant or analogue sequence of an amino acid sequence of the invention may differ on the basis of substitution (typically conservative substitution) deletion or insertion. Proteins comprising such variations are referred to herein as variants.

Proteins of the invention may include variants in which amino acid residues from one species are substituted for the corresponding residue in another species, either at the conserved or non-conserved positions. Variants of protein molecules disclosed herein may be produced and used in the present invention. Following the lead of computational chemistry in applying multivariate data analysis techniques to the structure/property-activity relationships [see for example, Wold, et al. Multivariate data analysis in chemistry. Chemometrics-Mathematics and Statistics in Chemistry (Ed.: B. Kowalski); D. Reidel Publishing Company, Dordrecht, Holland, 1984 (ISBN 90-277-1846-6] quantitative activity-property relationships of proteins can be derived using well-known mathematical techniques, such as statistical regression, pattern recognition and classification [see for example Norman et al. Applied Regression Analysis. Wiley-Interscience; 3rd edition (April 1998) ISBN: 0471170828; Kandel, Abraham et al. Computer-Assisted Reasoning in Cluster Analysis. Prentice Hall PTR, (May 11, 1995), ISBN: 0133418847; Krzanowski, Wojtek. Principles of Multivariate Analysis: A User's Perspective (Oxford Statistical Science Series, No 22 (Paper)). Oxford University Press; (December 2000), ISBN: 0198507089; Witten, Ian H. et al Data Mining: Practical Machine Learning Tools and Techniques with Java Implementations. Morgan Kaufmann; (Oct. 11, 1999), ISBN: 1558605525; Denison David G. T. (Editor) et al Bayesian Methods for Nonlinear Classification and Regression (Wiley Series in Probability and Statistics). John Wiley & Sons; (July 2002), ISBN: 0471490369; Ghose, Arup K. et al. Combinatorial Library Design and Evaluation Principles, Software, Tools, and Applications in Drug Discovery. ISBN: 0-8247-0487-8]. The properties of proteins can be derived from empirical and theoretical models (for example, analysis of likely contact residues or calculated physicochemical property) of proteins sequence, functional and three-dimensional structures and these properties can be considered individually and in combination.

Amino acids are referred to herein using the name of the amino acid, the three-letter abbreviation or the single letter abbreviation. The term “protein”, as used herein, includes proteins, polypeptides, and peptides. As used herein, the term “amino acid sequence” is synonymous with the term “polypeptide” and/or the term “protein”. In some instances, the term “amino acid sequence” is synonymous with the term “peptide”. The terms “protein” and “polypeptide” are used interchangeably herein. In the present disclosure and claims, the conventional one-letter and three-letter codes for amino acid residues may be used. The 3-letter code for amino acids as defined in conformity with the IUPACIUB Joint Commission on Biochemical Nomenclature (JCBN). It is also understood that a polypeptide may be coded for by more than one nucleotide sequence due to the degeneracy of the genetic code.

Amino acid residues at non-conserved positions may be substituted with conservative or non-conservative residues. In particular, conservative amino acid replacements are contemplated.

A “conservative amino acid substitution” is one in which the amino acid residue is replaced with an amino acid residue having a similar side chain. Families of amino acid residues having similar side chains have been defined in the art, including basic side chains (e.g., lysine, arginine, or histidine), acidic side chains (e.g., aspartic acid or glutamic acid), uncharged polar side chains (e.g., glycine, asparagine, glutamine, serine, threonine, tyrosine, or cysteine), nonpolar side chains (e.g., alanine, valine, leucine, isoleucine, proline, phenylalanine, methionine, or tryptophan), beta-branched side chains (e.g., threonine, valine, isoleucine) and aromatic side chains (e.g., tyrosine, phenylalanine, tryptophan, or histidine). Thus, if an amino acid in a polypeptide is replaced with another amino acid from the same side chain family, the amino acid substitution is considered to be conservative. The inclusion of conservatively modified variants in a protein of the invention does not exclude other forms of variant, for example polymorphic variants, interspecies homologs, and alleles.

“Non-conservative amino acid substitutions” include those in which (i) a residue having an electropositive side chain (e.g., Arg, His or Lys) is substituted for, or by, an electronegative residue (e.g., Glu or Asp), (ii) a hydrophilic residue (e.g., Ser or Thr) is substituted for, or by, a hydrophobic residue (e.g., Ala, Leu, Ile, Phe or Val), (iii) a cysteine or proline is substituted for, or by, any other residue, or (iv) a residue having a bulky hydrophobic or aromatic side chain (e.g., Val, His, Ile or Trp) is substituted for, or by, one having a smaller side chain (e.g., Ala or Ser) or no side chain (e.g., Gly).

“Insertions” or “deletions” are typically in the range of about 1, 2, or 3 amino acids. The variation allowed may be experimentally determined by systematically introducing insertions or deletions of amino acids in a protein using recombinant DNA techniques and assaying the resulting recombinant variants for activity. This does not require more than routine experiments for a skilled person.

A “fragment” of a polypeptide comprises at least 50%, at least 60%, at least 70%, at least 80%, at least 90%, at least 95%, at least 97% or more of the original polypeptide. For example, a fragment may comprise at least 5, at least 10, at least 11, at least 12, at least 13, at least 14, at least 15, at least 16, at least 17, at least 18, at least 19, at least 20, at least 30, at least 40, at least 50, at least 60, at least 70, at least 80, at least 90, at least 100, at least 150, at least 200, at least 250, at least 300, at least 350, at least 400 or more amino acids of the protein from which it is derived. Preferably a fragment may comprise no more than 50, no more than 60, no more than 70, no more than 80, no more than 90, no more than 100, no more than 150, no more than 200, no more than 250, no more than 300, no more than 350, or no more than 400 amino acids of the protein from which it is derived. A fragment may be continuous or discontinuous, preferably continuous.

The polynucleotides of the present invention may be prepared by any means known in the art. For example, large amounts of the polynucleotides may be produced by replication in a suitable host cell. The natural or synthetic DNA fragments coding for a desired fragment will be incorporated into recombinant nucleic acid constructs, typically DNA constructs, capable of introduction into and replication in a prokaryotic or eukaryotic cell. Usually the DNA constructs will be suitable for autonomous replication in a unicellular host, such as yeast or bacteria, but may also be intended for introduction to and integration within the genome of a cultured insect, mammalian, plant or other eukaryotic cell lines.

The polynucleotides of the present invention may also be produced by chemical synthesis, e.g. by the phosphoramidite method or the tri-ester method, and may be performed on commercial automated oligonucleotide synthesizers. A double-stranded fragment may be obtained from the single stranded product of chemical synthesis either by synthesizing the complementary strand and annealing the strand together under appropriate conditions or by adding the complementary strand using DNA polymerase with an appropriate primer sequence.

When applied to a nucleic acid sequence, the term “isolated” in the context of the present invention denotes that the polynucleotide sequence has been removed from its natural genetic milieu and is thus free of other extraneous or unwanted coding sequences (but may include naturally occurring 5′ and 3′ untranslated regions such as promoters and terminators), and is in a form suitable for use within genetically engineered protein production systems. Such isolated molecules are those that are separated from their natural environment.

In view of the degeneracy of the genetic code, considerable sequence variation is possible among the polynucleotides of the present invention. Degenerate codons encompassing all possible codons for a given amino acid are set forth below:

| Amino Acid | Codons | Degenerate Codon |
|---|---|---|
| Cys | TGC TGT | TGY |
| Ser | AGC AGT TCA TCC TCG TCT | WSN |
| Thr | ACA ACC ACG ACT | ACN |
| Pro | CCA CCC CCG CCT | CCN |
| Ala | GCA GCC GCG GCT | GCN |
| Gly | GGA GGC GGG GGT | GGN |
| Asn | AAC AAT | AAY |
| Asp | GAC GAT | GAY |
| Glu | GAA GAG | GAR |
| Gln | CAA CAG | CAR |
| His | CAC CAT | CAY |
| Arg | AGA AGG CGA CGC CGG CGT | MGN |
| Lys | AAA AAG | AAR |
| Met | ATG | ATG |
| Ile | ATA ATC ATT | ATH |
| Leu | CTA CTC CTG CTT TTA TTG | YTN |
| Val | GTA GTC GTG GTT | GTN |
| Phe | TTC TTT | TTY |
| Tyr | TAC TAT | TAY |
| Trp | TGG | TGG |
| Ter | TAA TAG TGA | TRR |
| Asn/Asp | | RAY |
| Glu/Gln | | SAR |
| Any | | NNN |

One of ordinary skill in the art will appreciate that flexibility exists when determining a degenerate codon, representative of all possible codons encoding each amino acid. For example, some polynucleotides encompassed by the degenerate sequence may encode variant amino acid sequences, but one of ordinary skill in the art can easily identify such variant sequences by reference to the amino acid sequences of the present invention.

A “variant” nucleic acid sequence has substantial homology or substantial similarity to a reference nucleic acid sequence (or a fragment thereof). A nucleic acid sequence or fragment thereof is “substantially homologous” (or “substantially identical”) to a reference sequence if, when optimally aligned (with appropriate nucleotide insertions or deletions) with the other nucleic acid (or its complementary strand), there is nucleotide sequence identity in at least about 70%, 75%, 80%, 85, 90, 91, 92, 93, 94, 95, 96, 97, 98, 99 or more % of the nucleotide bases. Methods for homology determination of nucleic acid sequences are known in the art.

Alternatively, a “variant” nucleic acid sequence is substantially homologous with (or substantially identical to) a reference sequence (or a fragment thereof) if the “variant” and the reference sequence they are capable of hybridizing under stringent (e.g. highly stringent) hybridization conditions. Nucleic acid sequence hybridization will be affected by such conditions as salt concentration (e.g. NaCl), temperature, or organic solvents, in addition to the base composition, length of the complementary strands, and the number of nucleotide base mismatches between the hybridizing nucleic acids, as will be readily appreciated by those skilled in the art. Stringent temperature conditions are preferably employed, and generally include temperatures in excess of 30° C., typically in excess of 37° C. and preferably in excess of 45° C. Stringent salt conditions will ordinarily be less than 1000 mM, typically less than 500 mM, and preferably less than 200 mM. The pH is typically between 7.0 and 8.3. The combination of parameters is much more important than any single parameter.

Methods of determining nucleic acid percentage sequence identity are known in the art. By way of example, when assessing nucleic acid sequence identity, a sequence having a defined number of contiguous nucleotides may be aligned with a nucleic acid sequence (having the same number of contiguous nucleotides) from the corresponding portion of a nucleic acid sequence of the present invention. Tools known in the art for determining nucleic acid percentage sequence identity include Nucleotide BLAST (as described below).

One of ordinary skill in the art appreciates that different species exhibit “preferential codon usage”. As used herein, the term “preferential codon usage” refers to codons that are most frequently used in cells of a certain species, thus favouring one or a few representatives of the possible codons encoding each amino acid. For example, the amino acid threonine (Thr) may be encoded by ACA, ACC, ACG, or ACT, but in mammalian host cells ACC is the most commonly used codon; in other species, different codons may be preferential. Preferential codons for a particular host cell species can be introduced into the polynucleotides of the present invention by a variety of methods known in the art. Introduction of preferential codon sequences into recombinant DNA can, for example, enhance production of the protein by making protein translation more efficient within a particular cell type or species.

A “fragment” of a polynucleotide of interest comprises a series of consecutive nucleotides from the sequence of said full-length polynucleotide. By way of example, a “fragment” of a polynucleotide of interest may comprise (or consist of) at least 600 consecutive nucleotides from the sequence of said polynucleotide (e.g. at least 600, 650, 700, 750, 800 850, 900, or 950 consecutive nucleic acid residues of said polynucleotide). Typically, a fragment as defined herein retains the same function as the full-length polynucleotide.

The terms “decrease”, “reduced”, “reduction”, or “inhibit” are all used herein to mean a decrease by a statistically significant amount. The terms “reduce,” “reduction” or “decrease” or “inhibit” typically means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” encompasses a complete inhibition or reduction as compared to a reference level. “Complete inhibition” is a 100% inhibition (i.e. abrogation) as compared to a reference level.

The terms “increased”, “increase”, “enhance”, or “activate” are all used herein to mean an increase by a statically significant amount. The terms “increased”, “increase”, “enhance”, or “activate” can mean an increase of at least 25%, at least 50% as compared to a reference level, for example an increase of at least about 50%, or at least about 75%, or at least about 80%, or at least about 90%, at least about 95%, or at least about 98%, or at least about 99%, or at least about 100%, or at least about 250% or more compared with a reference level, or at least about a 1.5-fold, or at least about a 2-fold, or at least about a 2.5-fold, or at least about a 3-fold, or at least about a 4-fold, or at least about a 5-fold or at least about a 10-fold increase, or any increase between 1.5-fold and 10-fold or greater as compared to a reference level. In the context of a yield or titre, an “increase” is an observable or statistically significant increase in such level.

The terms “individual”, “subject”, and “patient”, are used interchangeably herein to refer to a mammalian subject for whom diagnosis, prognosis, disease monitoring, treatment, therapy, and/or therapy optimisation is desired. The mammal can be (without limitation) a human, non-human primate, mouse, rat, dog, cat, horse, or cow. In a preferred embodiment, the individual, subject, or patient is a human. An “individual” may be an adult, juvenile or infant. An “individual” may be male or female.

A “subject in need” of treatment for a particular condition can be an individual having that condition, diagnosed as having that condition, or at risk of developing that condition.

A subject can be one who has been previously diagnosed with or identified as suffering from or having a condition in need of treatment or one or more complications or symptoms related to such a condition, and optionally, have already undergone treatment for a condition as defined herein or the one or more complications or symptoms related to said condition. Alternatively, a subject can also be one who has not been previously diagnosed as having a condition as defined herein or one or more or symptoms or complications related to said condition. For example, a subject can be one who exhibits one or more risk factors for a condition, or one or more or symptoms or complications related to said condition or a subject who does not exhibit risk factors.

As used herein, the term “healthy individual” refers to an individual or group of individuals who are in a healthy state, e.g. individuals who have not shown any symptoms of the disease, have not been diagnosed with the disease and/or are not likely to develop the disease e.g. malaria. Preferably said healthy individual(s) is not on medication affecting malaria and has not been diagnosed with any other disease. The one or more healthy individuals may have a similar sex, age, and/or body mass index (BMI) as compared with the test individual. Application of standard statistical methods used in medicine permits determination of normal levels of expression in healthy individuals, and significant deviations from such normal levels.

Herein the terms “control” and “reference population” are used interchangeably.

The term “pharmaceutically acceptable” as used herein means approved by a regulatory agency of the Federal or a state government, or listed in the U.S. Pharmacopeia, European Pharmacopeia or other generally recognized pharmacopeia

The publications discussed herein are provided solely for their disclosure prior to the filing date of the present application. Nothing herein is to be construed as an admission that such publications constitute prior art to the claims appended hereto.

Disclosure related to the various methods of the invention are intended to be applied equally to other methods, therapeutic uses or methods, the data storage medium or device, the computer program product, and vice versa.

### RH5-Interacting Protein (RIPR) Antigens

The present invention provides a vaccine composition comprising a Plasmodium RH5-Interacting Protein (RIPR) antigen. The term antigen or fragment thereof, means any peptide-based sequence that can be recognised by the immune system and/or that stimulates a cell-mediated immune response and/or stimulates the generation of antibodies.

Preferably the RIPR antigen is a fragment of the full length RIPR protein. The RIPR antigen typically induces an immune response (e.g., an antibody response) against the blood-stage Plasmodium falciparum parasite. Preferably, the RIPR antigen induces an increased immune response (e.g., an antibody response) against the blood-stage Plasmodium falciparum parasite compared with the full-length RIPR (e.g., SEQ ID NO: 1). All disclosure herein in relation to RIPR antigens of the invention applies equally and without reservation to RIPR fragments of the invention, and vice versa, unless expressly stated to the contrary.

RIPR is highly conserved, essential for invasion, and capable of inducing cross-strain neutralizing antibodies in assays of GIA. However, RIPR is not a target of naturally acquired immunity. RIPR is an approximately 120 kDa protein, first identified when gel filtration chromatography of what was presumed to be ion-exchange chromatography purified PfRH5 showed a 150-200 kDa compound, instead of the expected 45 kDa size of the processed fragment. Mass spectrometry revealed the presence of another protein, complexed with PfRH5, corresponding to RIPR. This interaction was confirmed by immunoprecipitation. Independently, RIPR was identified as a member of the Plasmodium falciparum ‘invadome’, a collection of proteins that were hypothesized to be involved in invasion. RIPR is composed of putatively unstructured regions, 10 epidermal growth factor-like (EGF) domains and has 87 cysteines. The global structure of RIPR is divided into an N-terminal portion and a C-terminal portion of approximately equal sizes. In the exemplary RIPR amino acid sequence of SEQ ID NO: 1, residues 716-900 (inclusive) correspond to EGF domains 5-8, residues 817-897 (inclusive) correspond to EGF domains 7-8 and residues 980-1086 (inclusive) correspond to the C-terminal domain (CTD). In view of the high level of sequence conservation between RIPR proteins from different plasmodium species, it is within the routine practice of one of ordinary skill in the art to identify the regions of other RIPR proteins which correspond to the EGF domains and C-terminal domain of the P. falciparum RIPR protein of SEQ ID NO: 1.

The RIPR may be any plasmodium RIPR. Typically, the RIPR is selected from any Plasmodium that infects humans. By way of non-limiting example, the RIPR may be a Plasmodium falciparum RIPR, a Plasmodium vivax RIPR, a Plasmodium malariae RIPR, a Plasmodium ovale curtisi RIPR, a Plasmodium ovale wallikeri RIPR or a Plasmodium knowlesi RIPR. Thus, when the RIPR antigen is a fragment of the full-length RIPR protein, the fragment may be a fragment of the full-length P. falciparum RIPR, P. vivax RIPR, P. malariae RIPR, P. ovale curtisi RIPR, P. ovale wallikeri RIPR or P. knowlesi RIPR. Preferably, the fragment is a fragment of P. falciparum RIPR. An exemplary P. falciparum RIPR amino acid sequence is defined by SEQ ID NO: 1 (Genbank XP_001351305, version 1, accessed 26 January 2023). Variants of this RIPR amino acid sequence are also encompassed, as described herein. A reference herein to a RIPR fragment of the invention may preferably refer to a fragment of SEQ ID NO: 1, or a variant thereof.

The RIPR antigen may comprise (or consist of) one or more RIPR epidermal-growth factor like (EGF) domain. Typically, the RIPR antigen comprises (or consists of) one, two, three, or four RIPR EGF domains. The RIPR EGF domains are preferably selected from the fifth, sixth, seventh, and eighth RIPR EGF domains. For example, the RIPR antigen may comprise (or consist of) the eighth RIPR EGF domain. The RIPR antigen may comprise (or consist of) the seventh RIPR EGF domain. The RIPR antigen may comprise (or consist of) the sixth RIPR EGF domain. The RIPR antigen may comprise (or consist of) the fifth RIPR EGF domain. The RIPR antigen may comprise (or consist of) the seventh and eighth RIPR EGF domains. The RIPR antigen may comprise (or consist of) the sixth, seventh and eighth RIPR EGF domains. The RIPR antigen may comprise (or consist of) the fifth, sixth, seventh and eighth RIPR EGF domains. A RIPR antigen of the invention may preferably comprise or consist of one or more RIPR EGF domains of SEQ ID NO: 1, or a variant thereof.

The wild type full-length 120 kDa RIPR protein is processed into two fragments of similar size, an N-terminal fragment (including EGF domains 1 and 2) and a C-terminal fragment (including EGF domains 3-10). The RIPR antigen may comprise (or consist of) a contiguous sequence from the C-terminal portion of the RIPR protein. Preferably, the RIPR antigen comprises (or consists of) a contiguous sequence from the C-terminal portion of the RIPR protein.

The RIPR antigen may comprise (or consist of) a contiguous sequence of no more than 400 amino acids from RIPR (e.g., SEQ ID NO: 1). For example, RIPR antigen may comprise (or consist of) no more than 300 amino acids, no more than 200 amino acids or no more than 100 amino acids from RIPR (e.g., SEQ ID NO: 1). Preferably, the contiguous sequence is from the C-terminal portion of RIPR.

The RIPR antigen may comprise (or consist of) a contiguous sequence of at least 50 amino acids from RIPR (e.g., SEQ ID NO: 1). For example, the RIPR antigen may comprise (or consist of) a contiguous sequence of at least 50, at least 60, at least 70, at least 80, at least 90, at least 100, at least 120, at least 140, at least 160, at least 180, at least 200, at least 220, at least 240, at least 260, at least 280 or at least 300 amino acids from RIPR (e.g., SEQ ID NO: 1). Preferably, the RIPR antigen comprises (or consists of) a contiguous sequence of at least 80, at least 160, at least 180, at least 260, or at least 280 amino acids from RIPR (e.g., SEQ ID NO: 1). The RIPR antigen may comprise (or consist of) a contiguous sequence from the C-terminal portion of the RIPR protein. Preferably, the RIPR antigen comprises (or consists of) a contiguous sequence from the C-terminal portion of the RIPR protein.

The RIPR antigen may comprise (or consist of) a contiguous sequence of from 50 to 400 amino acids from RIPR, from 60 to 350 amino acids from RIPR, or from 80 to 300 amino acids from RIPR (e.g., SEQ ID NO: 1). The RIPR antigen may comprise (or consist of) a contiguous sequence from the C-terminal portion of the RIPR protein. Preferably, the RIPR antigen comprises (or consists of) a contiguous sequence from the C-terminal portion of the RIPR protein.

The RIPR antigen may alternatively comprise a sequence having at least 80% sequence identity to a fragment of RIPR, as defined above. For example, the RIPR antigen may comprise a sequence having at least 85% sequence identity, at least 90% sequence identity, at least 95% sequence identity, at least 98% sequence identity, at least 99% sequence identity or 100% sequence identity to a fragment of RIPR, as defined above.

The RIPR antigen may comprise (or consist of) an amino acid sequence having at least 80% sequence identity to SEQ ID NO: 2. For example, the RIPR antigen may comprise a sequence having at least 85% sequence identity, at least 90% sequence identity, at least 95% sequence identity, at least 98% sequence identity, at least 99% sequence identity or 100% sequence identity to SEQ ID NO: 2.

The RIPR antigen may comprise (or consist of) amino acids corresponding to P716 to D900 of SEQ ID NO: 1. For example, the RIPR antigen may comprise a sequence having at least 85% sequence identity, at least 90% sequence identity, at least 95% sequence identity, at least 98% sequence identity, at least 99% sequence identity or 100% sequence identity to amino acids corresponding to P716 to D900 of SEQ ID NO: 1.

The RIPR antigen may comprise (or consist of) RIPR EGF domains 5-8, such as EGF domains 5-8 within SEQ ID NO:1.

The RIPR antigen may comprise (or consist of) an amino acid sequence having at least 80% sequence identity to SEQ ID NO: 3. For example, the RIPR antigen may comprise a sequence having at least 85% sequence identity, at least 90% sequence identity, at least 95% sequence identity, at least 98% sequence identity, at least 99% sequence identity or 100% sequence identity to SEQ ID NO: 3.

The RIPR antigen may comprise (or consist of) amino acids corresponding to D817 to V897 of SEQ ID NO: 1. For example, the RIPR antigen may comprise a sequence having at least 85% sequence identity, at least 90% sequence identity, at least 95% sequence identity, at least 98% sequence identity, at least 99% sequence identity or 100% sequence identity to amino acids corresponding to D817 to V897 of SEQ ID NO: 1. The RIPR antigen may comprises (or consists of) RIPR EGF domains 7-8, such as EGF domains 7-8 within SEQ ID NO:1.

The invention provides a RIPR antigen which is a fragment of the full-length RIPR protein and which comprises the C-terminal domain (CTD) of RIPR or a portion thereof. In some preferred embodiments, the RIPR antigen is a RIPR antigen of the invention as described herein which additionally comprises the C-terminal domain (CTD) of RIPR or a portion thereof.

As described herein, the RIPR CTD domain corresponds to residues 980-1086 (inclusive) of RIPR, the sequence of which is shown in SEQ ID NO: 4. Therefore, in some preferred embodiments, a RIPR antigen of the invention may comprise a CTD corresponding to SEQ ID NO: 4 or a fragment or variant thereof. For example, the RIPR antigen may comprise at least 80 amino acids, at least 90 amino acids or at least 100 amino acids from SEQ ID NO: 4. The inclusion of a CTD, or portion thereof, may induce non-neutralising antibodies. Preferably, the non-neutralising antibodies potentiate the GIA of antibodies raised against the RIPR antigen (e.g., EGF domains 5-8 or EGF domains 7-8).

The RIPR antigen may alternatively comprise a sequence having at least 80% sequence identity to SEQ ID NO: 4 or a portion thereof. For example, the RIPR antigen may comprise a sequence having at least 85% sequence identity, at least 90% sequence identity, at least 95% sequence identity, at least 98% sequence identity, at least 99% sequence identity or 100% sequence identity to SEQ ID NO: 4 or a portion thereof. Where the RIPR antigen comprises a sequence having sequence identity to a portion of SEQ ID NO: 4, the portion may be at least 80 amino acids, at least 90 amino acids or at least 100 amino acids from SEQ ID NO: 4.

The RIPR antigen may comprise (or consist of) an amino acid sequence having at least 80% sequence identity to SEQ ID NO: 5. For example, the RIPR antigen may comprise a sequence having at least 85% sequence identity, at least 90% sequence identity, at least 95% sequence identity, at least 98% sequence identity, at least 99% sequence identity or 100% sequence identity to SEQ ID NO: 5.

The RIPR antigen of the invention may comprise (or consist of): (a) a sequence having at least 80% sequence identity to SEQ ID NO: 14, or SEQ ID NO: 17 (preferably SEQ ID NO: 17) or (b) SEQ ID NO: 14 or SEQ ID NO: 17; preferably SEQ ID NO: 17.

The RIPR antigen of the invention may comprise (or consist of) a sequence having at least 80% sequence identity to SEQ ID NO: 2 and a sequence having at least 80% sequence identity SEQ ID NO: 4. The RIPR antigen of the invention may comprise (or consist of) SEQ ID NO: 2 and SEQ ID NO: 4. The sequences may be directly fused or connected by a linker.

The RIPR antigen of the invention may comprise (or consist of) a sequence having at least 80% sequence identity to SEQ ID NO: 3 and a sequence having at least 80% sequence identity SEQ ID NO: 4. The RIPR antigen of the invention may comprise (or consist of) SEQ ID NO: 3 and SEQ ID NO: 4. The sequences may be directly fused or connected by a linker.

The RIPR antigen may comprise (or consist of) an amino acid sequence having at least 80% sequence identity to SEQ ID NO: 6. For example, the RIPR antigen may comprise a sequence having at least 85% sequence identity, at least 90% sequence identity, at least 95% sequence identity, at least 98% sequence identity, at least 99% sequence identity or 100% sequence identity to SEQ ID NO: 6.

The RIPR antigen may lack one or more of RIPR EGF domain 1, 2, 3, 4, 9 or 10. The RIPR antigen may lack two or more of RIPR EGF domain 1, 2, 3, 4, 9 or 10. The RIPR antigen may lack three or more of RIPR EGF domain 1, 2, 3, 4, 9 or 10. The RIPR antigen may lack four or more of RIPR EGF domain 1, 2, 3, 4, 9 or 10. The RIPR antigen may lack five or more of RIPR EGF domain 1, 2, 3, 4, 9 or 10. The RIPR antigen may lack each of RIPR EGF domain 1, 2, 3, 4, 9 and 10. The RIPR antigen may lack RIPR EGF domains 1, 2, 3 and 4 and/or the RIPR antigen may lack RIPR EGF domains 9 and 10.

The RIPR antigen may lack an N-terminal portion of RIPR. For example, the RIPR antigen may lack the N-terminal 500 amino acids, the N-terminal 600 amino acids, the N-terminal 700 amino acids or the N-terminal 800 amino acids.

A RIPR antigen of the invention may be modified relative to the RIPR antigen from which it is derived, provided that the epitope defined herein is retained and the antigen still gives rise to a neutralising antibody.

Vaccine compositions comprising a RIPR antigen may induce antibodies that have reduced immunodominance compared to antibodies raised against full-length RIPR. Thus, the RIPR antigens of the invention may have a reduced inhibitory effect on the generation of antibodies to other components of the RCR complex. In other words, when combined with other antigens from the RCR complex, an individual vaccinated with a RIPR antigen of the invention may have an increased antibody response to other antigens from the RCR complex (e.g. CyRPA and/or RH5, or fragments thereof) compared with the antibody response of an individual who is vaccinated with a RIPR antigen of the prior art in combination with the same other antigens from the RCR complex. When combined with other antigens from the RCR complex, an individual vaccinated with a RIPR antigen of the invention may have a statistically significant increased antibody response to other antigens from the RCR complex (e.g. CyRPA and/or RH5, or fragments thereof) compared with the antibody response of an individual who is vaccinated with a RIPR antigen of the prior art in combination with the same other antigens from the RCR complex. For example, the vaccine compositions comprising a RIPR antigen may induce an antibody response to other antigens from the RCR complex (e.g. CyRPA and/or RH5, or fragments thereof) that is 2-fold, 3-fold, 4-fold, or 5-fold increased response compared with the antibody response of an individual who is vaccinated with a RIPR antigen of the prior art in combination with the same other antigens from the RCR complex.

Alternatively or in addition, vaccine compositions comprising a RIPR antigen may induce antibodies that have a higher proportion of inhibitory antibodies compared to full-length RIPR. For example, the vaccine compositions comprising a RIPR antigen may induce antibodies that have 2-fold, 2.5-fold, 3-fold, 4-fold, or 5-fold increased or more response relative to other RIPR antibodies. Increased response can be assessed by GIA per unit of total anti-RIPR antibody.

Alternatively or in addition, vaccine compositions comprising a RIPR antigen of the invention may induce antibodies that have increased growth inhibitory activity (GIA) against the blood-stage Plasmodium parasite compared to antibodies raised against full-length RIPR (e.g., relative to the full-length RIPR protein).

Typically, the RIPR antigen of the invention may induce antibodies which have a GIA of at least 40%, at least 50%, at least 60%, at least 70%, at least 80%, at least 90% or more against Plasmodium parasites. In a preferred embodiment, the RIPR antigens of the invention induce antibodies that have a growth inhibitory activity (GIA) of at least 70%, at least 75%, at least 80%, at least 90% or more against Plasmodium parasites.

The growth inhibitory activity (GIA) may be measured at any appropriate concentration of the antibodies, for example the GIA may be measured at 0.1 mg/ml, 0.2 mg/ml, 0.3 mg/ml, 0.4 mg/ml, 0.5 mg/ml, 0.6 mg/ml, 0.7 mg/ml, 0.8 mg/ml, 0.9 mg/ml, 1 mg/ml, 2 mg/ml, 3 mg/ml, 4 mg/ml, 5 mg/ml, 6 mg/ml, 7 mg/ml, 8 mg/ml, 9 mg/ml or 10 mg/ml of purified IgG antibody. The RIPR antigen of the invention may induce antibodies which give a GIA of at least 40%, at least 50% and preferably at least 70% or more against the blood-stage Plasmodium parasite, at an IgG concentration of 1 mg/ml IgG, of the purified antibody. Alternatively, the RIPR antigen of the invention may induce antibodies which give a GIA of at least 40%, at least 50% and preferably at least 70% or more against the blood-stage Plasmodium parasite, at an IgG concentration of 10 mg/ml IgG, of the purified antibody.

Vaccine compositions comprising a RIPR antigen of the invention (e.g. vaccine compositions comprising a fusion protein of the invention which comprises said RIPR antigen) may induce antibodies that have a growth inhibitory activity (GIA) of at least 50% against the blood-stage Plasmodium parasite. Any appropriate technique may be used to determine the GIA. Exemplary techniques are described in the examples and conventional techniques are known in the art.

Vaccine compositions comprising a RIPR antigen may induce antibodies that have a growth inhibitory activity (GIA) of at least 50% against a plurality of genetic strains of the blood-stage Plasmodium parasite. This is likely to be of importance in achieving protection against the variety of strains circulating in the natural environment. Accordingly, preferably, RIPR antigens of the invention induce antibodies that have a GIA of at least 50%, at least 60%, at least 70%, at least 80%, at least 90% or more against a plurality of genetic strains of the blood-stage Plasmodium parasite. In a preferred embodiment, the neutralising antibodies of the invention will result in a GIA of at least 70% or more against a plurality of genetic strains of the blood-stage Plasmodium parasite. Preferably, the genetic strain of the blood-stage Plasmodium parasite is Plasmodium falciparum.

A RIPR antigen of the invention may be provided in any appropriate form, e.g. as a (recombinant) protein, fusion protein, vector, DNA plasmid, RNA vaccine or other form, as described herein. In addition a RIPR antigen, and in particular a RIPR fusion protein (preferably a RIPR-CyRPA fusion protein) may be used in combination with one or more additional Plasmodium merozoite antigens which is independently provided in any appropriate form, e.g. as a (recombinant) protein, fusion protein, vector, DNA plasmid, RNA vaccine or other form, as described herein. Any and all disclosure in relation to RIPR antigens and RIPR fusion proteins of the invention applies equally and without reservation to any combination of a RIPR antigen, or a RIPR fusion protein of the invention and one or more additional Plasmodium merozoite antigens, regardless of the form of the RIPR antigen, RIPR fusion protein and/or the one or more additional Plasmodium merozoite antigens.

### Cysteine-Rich Protective Antigen (CyRPA) Antigen

CyRPA is a 43 kDa protein that was identified as a blood-stage vaccine candidate antigen through reverse vaccinology based on a predicted N-terminal secretion peptide, upregulation during the blood stage of infection, and a putative role in erythrocyte invasion owing to proximity to other invasion genes such as PfRH5. CyRPA has also been shown to coimmunoprecipitate with PfRH5 and RIPR. An exemplary P. falciparum CyRPA amino acid sequence is defined by SEQ ID NO: 7. Variants of this CyRPA amino acid sequence are also encompassed, as described herein. A reference herein to a CyRPA fragment of the invention may preferably refer to a fragment of SEQ ID NO: 7, or a variant thereof.

The CyRPA may be any Plasmodium CyRPA. Typically, the CyRPA is selected from any Plasmodium that infects humans. For example, the CyRPA may be a P. falciparum CyRPA, a P. vivax CyRPA, a P. malariae CyRPA, a P. ovale curtisi CyRPA, a P. ovale wallikeri CyRPA or a P. knowlesi CyRPA. Thus, when the CyRPA antigen is a fragment of the full length CyRPA protein, the fragment may be a fragment of the full-length P. falciparum CyRPA, P. vivax CyRPA, P. malariae CyRPA, P. ovale curtisi CyRPA, P. ovale wallikeri CyRPA or P. knowlesi CyRPA. Preferably, the fragment is a P. falciparum CyRPA.

The CyRPA antigen may comprises (or consists) of at least 200 amino acids, at least 225 amino acids, at least 250 amino acid, at least 275 amino acids, at least 300 amino acids, or at least 325 amino acids from CyRPA (e.g., SEQ ID NO: 7).

Preferably, the CyRPA antigen comprises or consists of SEQ ID NO: 7.

The CyRPA antigen may comprise (or consist of) a sequence having at least 80% sequence identity to SEQ ID NO: 7. For example, the CyRPA antigen may comprise a sequence having at least 85% sequence identity, at least 90% sequence identity, at least 95% sequence identity, at least 98% sequence identity, at least 99% sequence identity or 100% sequence identity to SEQ ID NO: 7 or a portion thereof. Where the CyRPA antigen comprises a sequence having sequence identity to a portion of SEQ ID NO: 7, the portion may be at least 200 amino acids, at least 225 amino acids, at least 250 amino acid, at least 275 amino acids, at least 300 amino acids, or at least 325 amino acids from SEQ ID NO: 7.

Vaccine compositions comprising a CyRPA antigen of the invention may induce antibodies against the blood-stage Plasmodium parasite.

Vaccine compositions comprising a CyRPA antigen may induce antibodies against a plurality of genetic strains of the blood-stage Plasmodium parasite. This is likely to be of importance in achieving protection against the variety of strains circulating in the natural environment.

A CyRPA antigen of the invention may be provided in any appropriate form, e.g. as a (recombinant) protein, fusion protein, vector, DNA plasmid, RNA vaccine or other form, as described herein. Preferably, the invention provides fusion proteins comprising a RIPR antigen of the invention and a CyRPA antigen of the invention, as described herein. Alternatively, a CyRPA antigen of the invention may be co-administered with a RIPR antigen of the invention, as described herein.

A CyRPA antigen, and in particular CyRPA fusion protein (preferably a RIPR-CyRPA fusion protein) may be used in combination with one or more additional Plasmodium merozoite antigens which is independently provided in any appropriate form, e.g. as a (recombinant) protein, fusion protein, vector, DNA plasmid, RNA vaccine or other form, as described herein.

Any and all disclosure in relation to CyRPA antigens and CyRPA fusion proteins of the invention applies equally and without reservation to any combination of a CyRPA antigen, or a CyRPA fusion protein of the invention and one or more additional Plasmodium merozoite antigens, regardless of the form of the CyRPA antigen, CyRPA fusion protein and/or the one or more additional Plasmodium merozoite antigens.

### Fusion Proteins

The invention also provides a vaccine composition comprising a fusion protein. The terms “fusion protein” and “fusion molecule” are used interchangeably and may refer to a polypeptide comprising a first immunogenic polypeptide (antigen or fragment thereof) linked to a one or more additional immunogenic polypeptide (antigen or fragment thereof). The first immunogenic polypeptide may be any RIPR antigen, such as a RIPR antigen of the invention as described herein, or a full-length RIPR protein. Preferably, the first immunogenic polypeptide is a RIPR antigen of the invention as described above. The one or more additional immunogenic polypeptide may preferably comprise or consist of another antigen or antigenic fragment from the RCR complex. In some particularly preferred embodiments, the one or more additional immunogenic polypeptide comprises or consists of a CyRPA antigen or fragment thereof. Thus, a fusion protein of the invention may comprise or consist of a RIPR antigen and a CyRPA antigen. Preferably, a fusion protein of the invention comprises or consists of a RIPR antigen as described herein and a CyRPA antigen as described herein. The one or more additional immunogenic polypeptide may comprise or consist of an RH5 antigen or fragment thereof. Further non-limiting examples of Plasmodium merozoite antigens that may be present in a fusion protein of the invention as one or more additional immunogenic polypeptide include PfAMA1, PfRON2, PfEBA175, PfRH1, PfRH2a, PfRH2b, PfRH4, PfRAP1, PfRAP3, PfMSRP5, PARAMA, PfSERA9, PfEBA181, PfCSS, PfPTRAMP and PfAARP, or fragments thereof. In particularly preferred embodiments, the CyRPA antigen induces an immune response (e.g., an antibody response) against the blood-stage P. falciparum parasite.

The polypeptide sequence of the first immunogenic polypeptide and the one or more additional immunogenic polypeptide may be covalently linked (e.g., fused) by recombinant, chemical or other suitable methods. The fusion molecule can be fused at one or several sites through a linker, particularly a peptide linker sequence, examples of which are described herein. Alternatively, the peptide linker may be used to assist in construction of the fusion molecule. Specifically preferred fusion molecules are fusion proteins. Generally fusion molecules also can be comprised of conjugate molecules.

Different orientations of the antigens are envisaged. Thus, the RIPR antigen may be at the N-terminus of the fusion protein and the one or more additional immunogenic polypeptide at the C-terminus. Alternatively, the RIPR antigen may be at the C-terminus of the fusion protein and the one or more additional immunogenic polypeptide at the N-terminus. Where there are two or more additional immunogenic polypeptides, the RIPR antigen may be positioned between the two or more additional immunogenic polypeptides. For example, the C-terminus of the CyRPA antigen may be N-terminal to the N-terminus of the RIPR antigen. Alternatively, the C-terminus of the RIPR antigen is N-terminal to the N-terminus of the CyRPA antigen.

The fusion protein may comprise (or consist of) a sequence having at least 80% sequence identity to any one of SEQ ID NO: 10 to SEQ ID NO: 13, SEQ ID NO: 15, SEQ ID NO: 16, SEQ ID NO: 18 and SEQ ID NO: 19. For example, the fusion protein antigen may comprise (or consist) of a sequence having at least 85% sequence identity, at least 90% sequence identity, at least 95% sequence identity, at least 98% sequence identity, at least 99% sequence identity or 100% sequence identity to any one of SEQ ID NO: 10 to SEQ ID NO: 13, SEQ ID NO: 15, SEQ ID NO: 16, SEQ ID NO: 18 and SEQ ID NO: 19.

The immunogenic polypeptides (e.g., the RIPR antigen and the CyRPA antigen) may be directly fused. Alternatively, the fusion protein may comprise a linker between the immunogenic polypeptides (e.g., the RIPR antigen and the CyRPA antigen). Preferably, the linker is a flexible linker (e.g., allowing effective positioning of the two immunogenic polypeptides). The flexible linker may, for example, be a peptide linker. The peptide linker may comprise (or consist of) 10, 11, 12, 13, 14, 15, 16, 17, 18, 19 or 20 amino acids. The peptide linker may comprise (or consist of) from 10 to 20 amino acids; preferably from 12 to 18 amino acids.

Additionally (or alternatively) the linker may be modified to facilitate the ligation to another polypeptide. Preferably, the linker comprises a 13 amino acid SPYTAG (e.g., SEQ ID NO: 8). For example, the linker comprising the SPYTAG may comprise the sequence of SEQ ID NO: 9.

The fusion protein may additionally comprise one or more further antigens. For example, the fusion protein may additionally comprise an antigen from the Reticulocyte binding Homologue family.

The Reticulocyte binding Homologue family comprises six members (PfRH1, PfRH2a, PfRH2b, PfRH3, PfRH4 and PfRH5), each of which is involved in the binding of the Plasmodium parasite to RBCs, with the possible exception of PfRH3 which may be a non-expressed pseudogene. The PfRH family has been identified as adhesins on the surface of the merozoite form of the Plasmodium parasite, which bind to receptors on the surface of the erythrocyte and hence permit invasion of RBCs by the parasite in its blood-stage. The PfRH5 antigen has an approximate molecular weight of 63 KDa. In vitro cleaved fragments of approximately 45 KDa and 28 KDa have been reported.

Fusion proteins of the invention may comprise a Reticulocyte binding protein Homologue 5 (PfRH5) antigen or a fragment thereof that induces antibodies which are highly effective in the GIA assay against the blood-stage Plasmodium parasite.

Vaccine compositions comprising the fusion protein of the invention may induce antibodies that have reduced immunodominance compared to antibodies raised against full-length RIPR and/or antibodies raised against the RIPR antigen of the invention. Vaccine compositions comprising the fusion protein of the invention may induce antibodies that have a higher proportion of inhibitory antibodies compared to full-length RIPR and/or antibodies raised against the RIPR antigen of the invention. Vaccine compositions comprising the fusion protein of the invention may induce antibodies that have increased growth inhibitory activity (GIA) against the blood-stage Plasmodium parasite compared to antibodies raised against full-length RIPR (e.g., relative to the full-length RIPR protein and/or relative to the RIPR antigen of the invention).

Vaccine compositions comprising the fusion protein of the invention may induce antibodies that have a growth inhibitory activity (GIA) of at least 50% against the blood-stage Plasmodium parasite. Vaccine compositions comprising the fusion protein of the invention may induce antibodies that have a growth inhibitory activity (GIA) of at least 50% is against a plurality of genetic strains of the blood-stage Plasmodium parasite. Preferably, the genetic strain of the blood-stage Plasmodium parasite is P. falciparum. Vaccine compositions comprising the fusion protein of the invention may induce antibodies that have a higher growth inhibitory activity (GIA) than the vaccine compositions comprising the RIPR antigen.

Co-Administration with a RH5 Antigen

The above-described vaccine composition (e.g., comprising the RIPR antigen or fusion protein of the invention, particularly a fusion protein of the invention comprising a RIPR antigen and a CyRPA antigen) may be co-administered with a Plasmodium falciparum RH5 antigen (PfRH5). The RIPR antigen/fusion protein and the PfRH5 antigen may be formulated in the same or separate vaccine compositions. When formulated as separate vaccine compositions, the vaccine composition comprising the RIPR antigen/fusion protein may be administered simultaneously or sequentially to the vaccine composition comprising the PfRH5 antigen.

Suitable PfRH5 antigens are described in detail in WO2012/114125, WO 2016/016651, WO 2018/055331, WO 2020/074908, each of which is incorporated by reference.

The PfRH5 antigens of the invention which give rise to anti-PfRH5 antibodies of the invention may be derived from unmodified PfRH5 fragments, from rationally designed PfRH5 fragments, or from optimised PfRH5 fragments.

The PfRH5 antigens of the invention may have variant sequences compared with the PfRH5 sequence (be it a wild-type/unmodified PfRH5 sequence, a rationally designed fragment or optimised PfRH5 sequence) from which it is derived. Such variants may exhibit at least 80%, at least 85%, at least 90%, at least 91%, at least 92%, at least 93%, at least 94%, at least 95%, at least 96%, at least 97% at least 98%, at least 99% or more identity with the PfRH5 sequence from which they are derived.

The PfRH5 sequence may comprise (or consist of) SEQ ID NO: 20. The PfRH5 sequence may comprise (or consist of) SEQ ID NO: 21. The PfRH5 sequence may comprise (or consist of) SEQ ID NO: 22. The PfRH5 sequence may comprise (or consist of) SEQ ID NO: 23. The PfRH5 sequence may comprise (or consist of) SEQ ID NO: 24. The PfRH5 sequence may comprise (or consist of) SEQ ID NO: 25. The PfRH5 sequence may comprise (or consist of) SEQ ID NO: 26. The PfRH5 sequence may comprise (or consist of) SEQ ID NO: 27. The PfRH5 sequence may comprise (or consist of) SEQ ID NO: 28. The PfRH5 sequence may comprise (or consist of) SEQ ID NO: 29. The PfRH5 sequence may comprise (or consist of) SEQ ID NO: 30. The PfRH5 sequence may comprise (or consist of) SEQ ID NO: 31. The PfRH5 sequence may comprise (or consist of) SEQ ID NO: 32. The PfRH5 sequence may comprise (or consist of) SEQ ID NO: 33. Preferably, the PfRH5 sequence comprises (or consists of) SEQ ID NO: 30 or SEQ ID NO: 31.

The PfRH5 antigen may comprise (or consists of) a sequence having at least 80% sequence identity to a PfRH5 sequence described herein. For example, the PfRH5 antigen may comprise a sequence having at least 85% sequence identity, at least 90% sequence identity, at least 95% sequence identity, at least 98% sequence identity, at least 99% sequence identity or 100% sequence identity to a PfRH5 sequence described herein. For example, the PfRH5 antigen may comprise a sequence having at least 85% sequence identity, at least 90% sequence identity, at least 95% sequence identity, at least 98% sequence identity, at least 99% sequence identity or 100% sequence identity to SEQ ID NO: 30 or SEQ ID NO: 31.

The above-described vaccine composition may additionally (or alternatively) be co-administered with a further P. falciparum antigen. For example, the antigens may be selected from the group consisting of PfAMA1, PfCSS, PHPTRAMP, PfEBA175, PfRH2a, PfRH2b or PfRH4, or a fragment thereof.

Co-Administration with a CyRPA Antigen

The above-described vaccine compositions comprising a RIPR antigen of the invention may be co-administered with a CyRPA antigen. The RIPR antigen and the CyRPA antigen may be formulated in the same or separate vaccine compositions. When formulated as separate vaccine compositions, the vaccine composition comprising the RIPA antigen may be administered simultaneously or sequentially to the vaccine composition comprising the CyRPA antigen.

### Suitable CyRPA Antigens are as Described Herein.

Additionally or alternatively, the above-described vaccine compositions comprising a RIPR antigen of the invention and a CyRPA antigen of the invention may be co-administered with a Plasmodium falciparum RH5 antigen (PfRH5). The RIPR antigen, CyRPA antigen and the PfRH5 antigen may be formulated in the same or separate vaccine compositions. For example, the RIPR antigen and the CyRPA antigen may be formulated in the same composition and the PfRH5 antigen may be formulated in a separate composition; the RIPR antigen and the PfRH5 antigen may be formulated in the same composition and the CyRPA antigen may be formulated in a separate composition; the CyRPA antigen and the PfRH5 antigen may be formulated in the same composition and the RIPR antigen may be formulated in a separate composition; each of the RIPR, CyRPA and PfRH5 antigens may be formulated in a separate composition; or the RIPR, CyRPA and PfRH5 antigens may be formulated in the same composition. When one or more of the RIPR, CyRPA and/or PfRH5 antigens is formulated as a separate vaccine composition, the individual vaccine compositions may be administered simultaneously or sequentially. By way of non-limiting example, the composition or compositions comprising the RIPR antigen and CyRPA antigen may be administered simultaneously or sequentially to the vaccine composition comprising the PfRH5 antigen.

Suitable PfRH5 antigens are as described herein.

The above-described vaccine composition may additionally (or alternatively) be co-administered with a further P. falciparum antigen. For example, the antigens may be selected from the group consisting of PfAMA1, PfCSS, PHPTRAMP, PfEBA175, PfRH2a, PfRH2b or PfRH4, or a fragment thereof.

### Antibodies

The present invention provides antibodies that bind specifically to the RIPR antigen or fusion proteins disclosed herein. Also provided are antibodies obtained following immunisation with the RIPR antigen or fusion proteins disclosed herein.

The following definitions are generally applicable to all antibodies of the invention.

The term “antibody”, as used herein, broadly refers to any immunoglobulin (Ig) molecule comprised of four polypeptide chains, two heavy (H) chains and two light (L) chains, or any functional fragment, mutant, variant, or derivation thereof, which retains the essential epitope binding features of an Ig molecule. Such mutant, variant, or derivative antibody entities are known in the art, non-limiting embodiments of which are discussed below.

In a full-length antibody, each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4.

Antibodies may be polyclonal (pAb) or monoclonal (mAb). Typically the antibodies of the invention are mAbs.

Antibodies of the invention can be of any type (e.g., IgG, IgE, IgM, IgD, IgA and IgY), class (e.g., IgG1, IgG2, IgG3, IgG4, IgAQ1 and IgA2) or subclass and may be from any species (e.g., mouse, human, chicken, rat, rabbit, sheep, shark and camelid).

The term “antigen-binding fragment” of an antibody (or simply “binding fragment”), as used herein, refers to one or more fragments of an antibody that retain the ability to specifically bind to an antigen. It has been shown that the antigen-binding function of an antibody can be performed by one or more fragments of a full-length antibody. Single chain antibodies are also encompassed. Such antigen-binding fragments may also be bispecific, dual specific, or multi-specific, specifically binding to two or more different antigens. Thus, examples of binding fragments encompassed within the term “antigen-binding fragment” of an antibody include Fab, Fv, scFv, dAb, Fd, Fab′ or F(ab′)2, tandem scFv and diabodies.

Also encompassed are antibody constructs, defined as a polypeptide comprising one or more the antigen binding fragment of the invention linked to a linker polypeptide or an immunoglobulin constant domain. Linker polypeptides comprise two or more amino acid residues joined by peptide bonds and are used to link one or more antigen binding portions.

An antibody of the invention may be a “human antibody”; defined as an antibody having variable and constant regions derived from human germline immunoglobulin sequences, but which may include amino acid residues not encoded by human germline immunoglobulin sequences (e.g., mutations introduced by random or site-specific mutagenesis in vitro or by somatic mutation in vivo), for example in the CDRs and in particular CDR3. Recombinant human antibodies are also encompassed by the present invention.

An antibody of the invention may be a “chimeric antibody”; defined as an antibody which comprises heavy and light chain variable region sequences from one species and constant region sequences from another species. The present invention encompasses chimeric antibodies having, for example, murine heavy and light chain variable regions linked to human constant regions.

An antibody of the invention may be a “CDR-grafted antibody”; defined as an antibody which comprise heavy and light chain variable region sequences from one species but in which the sequences of one or more of the CDR regions of VH and/or VL are replaced with CDR sequences of another species, such as antibodies having murine heavy and light chain variable regions in which one or more of the murine CDRs (e.g., CDR3 or all three CDRs) has been replaced with human CDR sequences.

An antibody of the invention may be a “humanized antibody”; defined as an antibody which comprise heavy and light chain variable region sequences from a non-human species (e.g., a mouse) but in which at least a portion of the VH and/or VL sequence has been altered to be more “human-like”, i.e., more similar to human germline variable sequences. One type of humanized antibody is a CDR-grafted antibody, in which human CDR sequences are introduced into non-human VH and VL sequences to replace the corresponding nonhuman CDR sequences.

The terms “Kabat numbering”, “Kabat definitions and “Kabat labeling” are used interchangeably herein. These terms, which are recognized in the art, refer to a system of numbering amino acid residues which are more variable (i.e. hypervariable) than other amino acid residues in the heavy and light chain variable regions of an antibody, or an antigen binding portion thereof (Kabat et al. (1971) Ann. NY Acad, Sci. 190:382-391 and Kabat, E. A., et al. (1991) Sequences of Proteins of Immunological Interest, Fifth Edition, U.S. Department of Health and Human Services, NIH Publication No. 91-3242).

Antibodies of the invention are not limited to a particular method of generation or production. Thus, the invention provides antibodies which have been manufactured from a hybridoma that secretes the antibody, as well as antibodies produced from a recombinantly produced cell that has been transformed or transfected with a nucleic acid or nucleic acids encoding the antibody. Such hybridomas, recombinantly produced cells, and nucleic acids form part of the invention.

An antibody (including full length antibodies or antigen-binding fragments thereof) of the invention may bind specifically to the RIPR antigen. By specifically, it will be understood that the antibody binds to the molecule of interest, in this case the RIPR antigen/epitope of the invention, with no significant cross-reactivity to any other molecule, particularly any other protein. For example, an antibody of the invention that is specific for a particular RIPR antigen/epitope of the invention will show no significant cross-reactivity with other epitopes.

As another example, an antibody of the invention that is specific for RIPR will show no significant cross-reactivity with other malarial invasion proteins. Cross-reactivity may be assessed by any suitable method. Cross-reactivity of an antibody of the invention for a RIPR epitope with another RIPR epitope or malarial invasion protein may be considered significant if the antibody binds to the other molecule at least 5%, 10%, 15%, 20%, 25%, 30%, 35%, 40%, 45%, 50%, 55%, 60%, 65%, 70%, 75%, 80%, 85%, 90% or 100% as strongly as it binds to the RIPR epitope. An antibody that is specific for the RIPR fragment may bind to another molecule such as PfRH5 or PfAMA1 at less than 90%, 85%, 80%, 75%, 70%, 65%, 60%, 55%, 50%, 45%, 40%, 35%, 30%, 25% or 20% the strength that it binds to the RIPR epitope. Preferably, the antibody binds to the other molecule at less than 20%, less than 15%, less than 10% or less than 5%, less than 2% or less than 1% the strength that it binds to the RIPR epitope. Binding affinity may be quantified in any suitable way, e.g. by K_{D}.

The binding affinity of a RIPR antibody of the invention for RIPR may be quantified in terms of dissociation constant (K_{D}). K_{D} may be determined using any appropriate technique, but SPR is generally preferred in the context of the present invention. A RIPR antibody of the invention may bind to RIPR with a K_{D} of less than 1 μM, less than 100 nM, less than 50 nM, less than 25 nM, less than 10 nM, less than 1 nM, less than 900 μM, less than 800 μM, less than 700 μM, less than 600 μM, less than 500 μM, less than 400 μM, less than 300 μM, less than 200 μM, less than 100 μM, less than 50 μM, less than 25 μM, less than 10 μM, less than 5 μM, or less. Typically a RIPR antibody of the invention binds to RIPR with a K_{D} of less 50 nM, less than 10 nM or less than 1 nM.

Any disclosure relating to RIPR antibodies equally applies to antibodies that bind to isolated RIPR antigen antibodies and antibodies that bind to the fusion protein of the invention (e.g., a RIPR antigen within the context of the fusion protein). Thus, by way of non-limiting example, an antibody of the invention may bind to a RIPR fusion protein with a K_{D} of less than 1 μM, less than 100 nM, less than 50 nM, less than 25 nM, less than 10 nM, less than 1 nM, less than 900 μM, less than 800 μM, less than 700 μM, less than 600 μM, less than 500 μM, less than 400 μM, less than 300 μM, less than 200 μM, less than 100 μM, less than 50 μM, less than 25 μM, less than 10 μM, less than 5 μM, or less. Typically a RIPR antibody of the invention binds to RIPR with a K_{D} of less 50 nM, less than 10 nM or less than 1 nM.

Further antibodies which bind to the epitopes/antigens of the invention may be generated by producing variants of the antibodies of the invention. Such variants may have CDRs sharing a high level of identity with the CDRs of an antibody of the invention, for example may have CDRs each of which independently may differ by one or two amino acids from the antibody of the invention from which the variant antibody is derived, and wherein the variant retains the binding and functional properties of the antibody of the invention. Additionally, such antibodies may have one or more variations (e.g. a conservative amino acid substitution) in the framework regions. Conservative amino acid substitutions are particularly contemplated (see the disclosure herein in relation to RIPR antigen variants, which applies equally to variants of antibodies of the invention). The variations in the amino acid sequences of the antibodies of the invention should maintain at least 75%, at least 80%, at least 85%, at least 90%, at least 95% and up to 99% sequence identity. Variants having at least 90% sequence identity with the antibodies of the invention, particularly the specific antibodies exemplified herein are specifically contemplated. Variation may or may not be limited to the framework regions and not present in the CDRs.

DNA oligonucleotide aptamers, RNA oligonucleotide aptamers, and other engineered biopolymers against a RIPR antigen of the invention may also be able to replicate the activity of the antibodies and combinations thereof described here. As a vaccine, RIPR antigens of the invention are likely amenable to expression by recombinant viral vectored vaccines, as well as nucleic acid-based vaccines such as RNA or DNA; and recombinant protein or virus-like particles (VLPs) expressed in mammalian expression systems or insect cell systems. It is also possible to express the RIPR antigens of the invention as proteins or VLPs in bacteria or yeast, as well as plant/algae systems.

Preferably, antibodies of the invention inhibit the growth of P. falciparum parasites, and more preferably across a plurality of strains of blood-stage P. falciparum parasites. This activity may be quantified using any appropriate technique and measured in any appropriate units.

Typically, antibodies of the invention have a GIA of at least at least 40%, at least 50%, at least 60%, at least 70%, at least 80%, at least 90% or more against Plasmodium parasites. In a preferred embodiment, the antigens of the invention induce antibodies that have a growth inhibitory activity (GIA) of at least 70%, at least 75%, at least 80%, at least 90% or more against Plasmodium parasites.

The growth inhibitory activity (GIA) may be measured at any appropriate concentration of the antibodies. For example, the antibodies of the invention may give a GIA of at least 40%, at least 50% and preferably at least 70% or more against the blood-stage Plasmodium parasite, at an IgG concentration of 1 mg/ml IgG, of the purified antibody.

An antibody of the invention typically has an GIA EC₅₀ which is at least comparable to total IgG against full length RIPR. Typically an antibody of the invention has an EC₅₀ value of less than 500 μg/ml, less than 400 μg/ml, less than 300 μg/ml, less than 200 μg/ml, less than 150 μg/ml, less than 100 μg/ml, less than 90 μg/ml, less than 80 μg/ml, less than 70 μg/ml, less than 60 μg/ml, less than 50 μg/ml, less than 40 μg/ml 1, less than 30 μg/ml, less than 25 μg/ml, less than 20 μg/ml, less than 15 μg/ml, less than 10 μg/ml, less than 9 μg/ml, less than 8 μg/ml, less than 7 μg/ml, less than 6 μg/ml, less than 5 μg/ml, less than 4 μg/ml, less than 3 μg/ml, less than 2 μg/ml, less than 1 μg/ml, or less.

Typically the antibodies of the invention result in a GIA of at least 50%, at least 60%, at least 70%, at least 80%, at least 90% or more against the blood-stage Plasmodium parasite. In a preferred embodiment, the antibodies of the invention will raise antibodies that result in a GIA of at least 70% against the blood-stage Plasmodium parasite measured at any appropriate concentration as described herein.

Typically the antibodies of the invention are effective against genetically diverse strains of the Plasmodium parasite. This is likely to be of importance in achieving protection against the variety of strains circulating in the natural environment. Accordingly, in a preferred embodiment, the antibodies of the invention will result in a GIA of at least at least 50%, at least 60%, at least 70%, at least 80%, at least 90% or more against a plurality of genetic strains of the blood-stage Plasmodium parasite. In a preferred embodiment, the antibodies of the invention will result in a GIA of at least 70% or more against a plurality of genetic strains of the blood-stage Plasmodium parasite.

### Vectors and Plasmids

The present invention provides vectors that express a RIPR antigen or fusion polypeptide of the invention. Typically the vector is present in the form of a vaccine formulation.

The vector encoding RIPR antigen or fusion polypeptide of the invention may be co-administered with one or more vector encoding one or more additional antigens. Preferably the one or more additional antigens is a Plasmodium merozoite antigen. For example, the one or more additional antigens, typically one or more additional Plasmodium merozoite antigen, may be selected from PfRH5, PfCyRPA, PfP113, PfRhopH3, PfRAP2, PfAMA1, PfRON2, PfEBA175, PfRH1, PfRH2a, PfRH2b, PfRH4, PfRAP1, PfRAP3, PfMSRP5, PfRAMA, PfSERA9, PfEBA181, PfCSS, PHPTRAMP and PfAARP, or a fragment thereof. The vector(s) may be present in the form of a vaccine formulation.

Also provided is a kit comprising a first vector that expresses a RIPR antigen or fusion polypeptide of the invention and a second vector that expresses one or more additional antigens, which may be selected from PfRH5, PfCyRPA, PfP113, PfRhopH3, PfRAP2, PfAMA1, PfRON2, PfEBA175, PfRH1, PfRH2a, PfRH2b, PfRH4, PfRAP1, PfRAP3, PfMSRP5, PfRAMA, PfSERA9, PfEBA181, PfCSS, PfPTRAMP and PfAARP.

The vector encoding RIPR antigen or fusion polypeptide of the invention may further express one or more additional antigens. Preferably the one or more additional antigens is a Plasmodium merozoite antigen. For example, the one or more additional antigens, typically one or more additional Plasmodium merozoite antigen, may be selected from PfRH5, PfCyRPA, PfP113, PfRhopH3, PfRAP2, PfAMA1, PfRON2, PfEBA175, PfRH1, PfRH2a, PfRH2b, PfRH4, PfRAP1, PfRAP3, PfMSRP5, PARAMA, PfSERA9, PfEBA181, PfCSS, PfPTRAMP and PfAARP, or a fragment thereof. The vector may be present in the form of a vaccine formulation. In some preferred embodiments, the vector further expresses a PfRH5 antigen, such as a PfRH5 antigen as described herein.

The vector(s) may be a viral vector. Such a viral vector may be an adenovirus (of a human serotype such as AdHu5, a simian serotype such as ChAd63, ChAdOX1 or ChAdOX2, or another form), an adeno-associated virus (AAV), or poxvirus vector (such as a modified vaccinia Ankara (MVA)). ChAdOX1 and ChAdOX2 are disclosed in WO2012/172277 (herein incorporated by reference in its entirety). ChAdOX2 is a BAC-derived and E4 modified AdC68-based viral vector. Preferably said viral vector is an AAV vector.

Viral vectors are usually non-replicating or replication impaired vectors, which means that the viral vector cannot replicate to any significant extent in normal cells (e.g. normal human cells), as measured by conventional means—e.g. via measuring DNA synthesis and/or viral titre. Non-replicating or replication impaired vectors may have become so naturally (i.e. they have been isolated as such from nature) or artificially (e.g. by breeding in vitro or by genetic manipulation). There will generally be at least one cell-type in which the replication-impaired viral vector can be grown—for example, modified vaccinia Ankara (MVA) can be grown in CEF cells. In one embodiment, the vector is selected from a human or simian adenovirus or a poxvirus vector.

Typically, the viral vector is incapable of causing a significant infection in an animal subject, typically in a mammalian subject such as a human or other primate.

The vector(s) may be a DNA vector, such as a DNA plasmid. The DNA vector(s) is typically capable of expression in a mammalian cell expression system, such as an immunised cell. The vector may be suitable for expression in a bacterial and/or insect host cell or expression system, such as any of those exemplified herein. A non-limiting example of a suitable expression vector is a pET15b vector, which may be optionally modified to encode an N-terminal tag, such as a hexa-histidine tag and/or a protease cleavage site, such as a TEV protease cleavage site.

The vector(s) may be a RNA vector, such as a self-amplifying RNA vaccine (Geall, A. J. et al., Proc Natl Acad Sci USA 2012; 109 (36) pp. 14604-9; incorporated herein by reference).

The present invention may be a phage vector, such as an AAV/phage hybrid vector as described in Hajitou et al., Cell 2006; 125 (2) pp. 385-398; herein incorporated by reference.

Vectors of the present invention also include virus-like particles (VLP), soluble proteins and/or fusion proteins comprising the antigens as described herein. Methods for generating VLPs are known in the art (see, for example, Brune et al. Sci. Rep. (2016), 19 (6): 19234, which is incorporated by reference in its entirety) and can readily be applied to the present invention. References herein to vectors of the invention may apply equally to VLP, soluble proteins and/or fusion proteins of the invention.

The epitopes/antigens of the invention may be expressed by in a single vector. Wherein the vector of the invention expresses a RIPR antigen or fusion polypeptide of the invention, the expressed RIPR antigen or fusion polypeptide is capable of inducing both neutralising antibodies (or other binding proteins) against RIPR, as described herein. Alternatively, the RIPR antigen or fusion polypeptide of the invention and the one or more additional antigen or fragment thereof may be delivered using a mixture of vectors expressing the individual epitopes/antigens (Forbes, E. K., et al., J Immunol, 2011. 187 (7): p. 3738-50; and Sheehy, S. H., et al., Mol Ther, 2012. 20 (12): p. 2355-68; both of which are incorporated herein by reference). Where the RIPR antigen or fusion polypeptide and one or more additional antigen or fragment thereof are co-expressed, this may be in the form of a fusion protein (Porter, D. W., et al., Vaccine, 2011. 29 (43): p. 7514-22; incorporated herein by reference), or the RIPR antigen or fusion polypeptide and one or more additional antigen or fragment thereof expressed as separate transcripts under the control of separate promoters (Bruder, J. T., et al., Vaccine, 2010. 28 (18): p. 3201-10; and Tine, J. A., et al., Infect Immun, 1996. 64 (9): p. 3833-44; both of which are incorporated herein by reference), or the RIPR antigen or fusion polypeptide and one or more additional antigen or fragment thereof may be translated as a single polypeptide which undergoes cleavage to yield two separate antigens (Ibrahimi, A., et al., Hum Gene Ther, 2009. 20 (8): p. 845-60; incorporated herein by reference).

The epitopes/antigens and fusion proteins of the invention may include a leader sequence, for example to assist in recombinant production and/or secretion. Any suitable leader sequence may be used, including conventional leader sequences known in the art. Suitable leader sequences include Bip leader sequences, which are commonly used in the art to aid secretion from insect cells and human tissue plasminogen activator leader sequence (tPA), which is routinely used in viral and DNA based vaccines and for protein vaccines to aid secretion from mammalian cell expression platforms. By way of a non-limiting example, the epitopes/antigens and fusion proteins of the invention may include the secretory signal from bovine tissue plasminogen activator, or may include another signal to direct the subcellular trafficking of the epitopes/antigens and fusion proteins. Alternatively, the epitopes/antigens and fusion proteins of the invention may be the mature form in which the N-terminal signal peptide has been removed.

The epitopes/antigens and fusion proteins of the invention may additionally comprise an N- or C-terminal tag, for example to assist in recombinant production and/or purification. Any N- or C-terminal tag may be used, including conventional tags known in the art. Suitable tags sequences include C-terminal hexa-histidine tags and the “C-tag” (the four amino acids EPEA at the C-terminus), which are commonly used in the art to aid purification from heterologous expression systems, e.g. insect cells, mammalian cells, bacteria, or yeast. Other examples of suitable tags include GST and MBP tags, or any other conventional tag which may be used to facilitate increased expression. In other embodiments, the epitopes/antigens and fusion proteins of the invention are purified from heterologous expression systems without the need to use a purification tag.

### Expression Systems

The RIPR antigen, fusion protein or antibody of the invention may be expressed using any suitable systems.

Generation of antibodies once an epitope has been identified can be carried out using known methods in the art. The antibodies per se can be generated using such methods as mouse hybridomas, phage display or ribosome display. Other methods known to the person skilled in the art may also be used. The resultant functional antibodies from a mouse immunisation or a phage display screen can then have their binding confirmed by epitope mapping. Accordingly, the invention provides a hybridoma expressing any antibody (non-neutralising or neutralising) of the invention.

For example, one could readily determine whether an antibody binds to the epitope of the invention via a reference anti-RIPR antibody. Such methods are a matter of routine in the art. Such experiments can be carried out using ELISA, RIA, Biacore, flow cytometry or other known antibody binding assays.

Antigens of the invention may be expressed using conventional systems. Such a system may be a prokaryotic or a eukaryotic system. Examples of such systems are well-known in the art. Non-limiting examples of suitable host systems include Escherichia coli, Saccharomyces cerevisiae, Pichia pastoris, non-lytic insect cell expression systems such as Schneider 2 (S2) and Schneider 3 (S3) cells from Drosophila melanogaster and Sf9 and Sf21 cells from Spodoptera frugiperda, and mammalian expression systems such as CHO cells and human embryonic kidney (HEK/HEK293) cells.

Accordingly, the invention provides a host cell containing a recombinant expression vector which encodes for an antigen or fusion protein of the invention. In a preferred embodiment the host cell is an insect cell, preferably a Drosophila melanogaster cell, or a Pichia yeast cell, or an Escherichia coli cell.

The term antigen as used herein refers to any peptide-based sequence that can be recognised by the immune system and/or that stimulates a cell-mediated immune response and/or stimulates the generation of antibodies. The antigens of the invention may be present in the form of a vaccine composition or vaccine formulation.

As described herein, the antigens of the invention raise antibodies as described herein. The antibodies raised by antigens of the invention inhibit the growth of malarial parasites, i.e. Plasmodium parasites, preferably across a plurality of strains of blood-stage Plasmodium parasites. In a more preferred embodiment, the antigens of the invention raise antibodies that inhibit the growth of Plasmodium falciparum parasites, and more preferably across a plurality of strains of blood-stage P. falciparum parasites. The effectiveness of the antigens of the invention may be quantified using any appropriate technique and measured in any appropriate units. For example, the effectiveness of the antigens of the invention may be given in terms of their growth inhibitory activity (GIA), half maximal effective concentration (EC₅₀), antibody titre stimulated (in terms of antibody units, AU) and/or EC₅₀ in terms of AU (described herein in relation to antibodies of the invention). The latter of these gives an indication of the quality of the antibody response stimulated by the antigen of the invention. Any appropriate technique may be used to determine the GIA, EC₅₀, AU or EC₅₀/AU. Exemplary techniques are described in the examples and conventional techniques are known in the art. The disclosure herein in relation to quantifying the effectiveness of the antibodies of the invention applies equally to antibodies raised by antigens or epitopes of the invention.

The antigen (e.g., RIPR antigen) or fusion protein of the invention may be expressed by a single vector. The antigens (e.g., RIPR antigen) or fusion protein may alternatively be delivered using vaccine platforms such as particle-based protein vaccine delivery (Bachmann, M. F., et al., Nat Rev Immunol, 2010. 10 (11): p. 787-96; incorporated herein by reference), or virus-like particles (VLP). Preferably the RIPR antigen is fused to a CyRPA antigen, thereby forming a particle and/or enhanced immunogenicity.

### Therapeutic Indications

The present invention also provides a method of stimulating or inducing an immune response in a subject comprising administering to the subject one or more composition, antigen (e.g., RIPR antigen), fusion protein (e.g., RIPR-CyRPA fusion), antibody or vector (e.g., a viral vector, RNA vaccine or DNA plasmid) of the invention (as described above).

Thus, the invention provides a vaccine composition comprising one or more antigen (e.g., RIPR antigen), fusion protein (e.g., RIPR-CyRPA fusion), antibody or vector (e.g., a viral vector, RNA vaccine or DNA plasmid) of the invention.

The method of stimulating or inducing an immune response in a subject may comprise administering one or more composition, antigen (e.g., RIPR antigen), fusion protein (e.g., RIPR-CyRPA fusion), antibody or vector (e.g., a viral vector, RNA vaccine or DNA plasmid) of the invention to a subject.

In the context of the therapeutic uses and methods, a “subject” is any animal subject that would benefit from stimulation or induction of an immune response against a Plasmodium parasite. Typical animal subjects are mammals, such as primates, for example, humans.

Thus, the present invention provides a method for treating or preventing malaria.

The present invention also provides a composition, antigen (e.g., RIPR antigen), fusion protein (e.g., RIPR-CyRPA fusion), antibody or vector (e.g., a viral vector, RNA vaccine or DNA plasmid) of the invention for use in the prevention or treatment of malaria.

The present invention also provides the use of a composition, antigen (e.g., RIPR antigen), fusion protein (e.g., RIPR-CyRPA fusion), antibody or vector (e.g., a viral vector, RNA vaccine or DNA plasmid) of the invention in the manufacture of a medicament for the prevention and/or treatment of malaria.

When used in a therapeutic application as described herein, a RIPR antigen of the invention may be provided in any appropriate form, e.g. as a (recombinant) protein, fusion protein, vector, DNA plasmid, RNA vaccine or other form, as described herein. In addition, when used in a therapeutic application as described herein, a RIPR antigen, and in particular a RIPR fusion protein (preferably a RIPR-CyRPA fusion protein) may be used in combination with one or more additional Plasmodium merozoite antigens which is independently provided in any appropriate form, e.g. as a (recombinant) protein, fusion protein, vector, DNA plasmid, RNA vaccine or other form, as described herein. Any and all disclosure in relation to therapeutic applications of RIPR antigens and RIPR fusion proteins of the invention applies equally and without reservation to any combination of a RIPR antigen, or a RIPR fusion protein of the invention and one or more additional Plasmodium merozoite antigens, regardless of the form of the RIPR antigen, RIPR fusion protein and/or the one or more additional Plasmodium merozoite antigens.

Thus, the one or more antigens of the invention (e.g., RIPR and optionally also CyRPA) may be in the form of a recombinant protein, a protein particle, a virus-like particle, a fusion protein, or a combination thereof as described herein. In addition, the one or more antigens of the invention may be used in combination with one or more further antigens selected from the group consisting of PfCyRPA, PfRH5, PfP113, PfRhopH3, PfRAP2, PfAMA1, PfRON2, PfEBA175, PfRH1, PfRH2a, PfRH2b, PfRH4, PfRAP1, PfRAP3, PfMSRP5, PfRAMA, PfSERA9, PfEBA181, PfCSS, PAPTRAMP and PfAARP, or a fragment thereof; for use in prevention or treatment of malaria.

The present invention provides the use of a composition, antigen (e.g., RIPR antigen), fusion protein (e.g., RIPR-CyRPA fusion), antibody or vector (e.g., a viral vector, RNA vaccine or DNA plasmid) of the invention either alone or in combination in the prevention or treatment of malaria.

The method for treating or preventing malaria may comprise administering a therapeutically effective amount of a composition, antigen (e.g., RIPR antigen), fusion protein (e.g., RIPR-CyRPA fusion), antibody or vector (e.g., a viral vector, RNA vaccine or DNA plasmid) of the invention either alone or in combination, to a subject.

As used herein, the term “treatment” or “treating” embraces therapeutic or preventative/prophylactic measures, and includes post-infection therapy and amelioration of malaria.

As used herein, the term “preventing” includes preventing the initiation of malaria and/or reducing the severity or intensity of malaria. The term “preventing” includes inducing or providing protective immunity against malaria. Immunity to malaria may be quantified using any appropriate technique, examples of which are known in the art.

A composition comprising an antigen (e.g., RIPR antigen), fusion protein (e.g., RIPR-CyRPA fusion), antibody or vector (e.g., a viral vector, RNA vaccine or DNA plasmid) of the invention may be administered to a subject (typically a mammalian subject such as a human or other primate) already having malaria, a condition or symptoms associated with malaria, to treat or prevent malaria. For example, the subject may be suspected of having come in contact with Plasmodium parasite, or has had known contact with Plasmodium parasite, but is not yet showing symptoms of exposure.

When administered to a subject (e.g. a mammal such as a human or other primate) that already has malaria, or is showing symptoms associated with Plasmodium parasite infection, a composition, antigen (e.g., RIPR antigen), fusion protein (e.g., RIPR-CyRPA fusion), antibody or vector (e.g., a viral vector, RNA vaccine or DNA plasmid) of the invention can cure, delay, reduce the severity of, or ameliorate one or more symptoms, and/or prolong the survival of a subject beyond that expected in the absence of such treatment.

Alternatively, a composition comprising an antigen (e.g., RIPR antigen), fusion protein (e.g., RIPR-CyRPA fusion), antibody or vector (e.g., a viral vector, RNA vaccine or DNA plasmid) of the invention may be administered to a subject (e.g. a mammal such as a human or other primate) who ultimately may be infected with Plasmodium parasite, in order to prevent, cure, delay, reduce the severity of, or ameliorate one or more symptoms of malaria, or in order to prolong the survival of a subject beyond that expected in the absence of such treatment, or to help prevent that subject from transmitting malaria.

The treatments and preventative therapies of the present invention are applicable to a variety of different subjects of different ages. In the context of humans, the therapies are applicable to children (e.g. infants, children under 5 years old, older children or teenagers) and adults. In the context of other animal subjects (e.g. mammals such as primates), the therapies are applicable to immature subjects and mature/adult subjects.

The present invention provides vaccine compositions comprising an antigen (e.g., RIPR antigen), fusion protein (e.g., RIPR-CyRPA fusion), antibody or vector (e.g., a viral vector, RNA vaccine or DNA plasmid) of the invention. Said vaccine compositions may further comprise one or more additional malarial antigens (Plasmodium merozoite antigen) as described herein, and/or any further components as described herein. The one or more additional Plasmodium merozoite antigens which is independently provided in any appropriate form, e.g. as a (recombinant) protein, fusion protein, vector, DNA plasmid, RNA vaccine or other form, as described herein.

A vaccine composition of the invention may further comprise one or more additional antigens selected from the group consisting of PfCyRPA, PfRH5, PfP113, PfRhopH3, PfRAP2, PfAMA1, PfRON2, PfEBA175, PfRH1, PfRH2a, PfRH2b, PfRH4, PfRAP1, PfRAP3, PfMSRP5, PfRAMA, PfSERA9, PfEBA181, PfCSS, PHPTRAMP and PfAARP, or a fragment thereof. A vaccine composition of the invention may further comprise one or more vectors expressing one or more additional antigen selected from the group consisting of PfCyRPA, PfRH5, PfP113, PfRhopH3, PfRAP2, PfAMA1, PfRON2, PfEBA175, PfRH1, PfRH2a, PfRH2b, PfRH4, PfRAP1, PfRAP3, PfMSRP5, PARAMA, PfSERA9, PfEBA181, PfCSS, PfPTRAMP and PfAARP, or a fragment thereof. These additional antigens may be expressed in any suitable form. As a non-limiting example, expression may be as a virus like particle (VLP). Recombinant particulate vaccines are well known in the art. They may be, for example, either fusion proteins or proteins chemically conjugated to particles. Examples of fusion proteins are hepatitis B surface antigen fusions (e.g. as in the RTS,S malaria vaccine candidate), hepatitis B core antigen fusions, or Ty-virus like particles. Examples of chemical fusion particles are the Q-beta particles under development by the biotechnology company Cytos (Zurich, Switzerland) and as in Brune et al. Sci. Rep. (2016), 19 (6): 19234.

The composition comprising an antigen (e.g., RIPR antigen), fusion protein (e.g., RIPR-CyRPA fusion), antibody or vector (e.g., a viral vector, RNA vaccine or DNA plasmid) of the invention may be combined with a composition comprising an additional antigen, e.g. by mixing two separate vaccines, or by co-delivery using vaccine platforms such as particle-based protein vaccine delivery, or by using a mixture of viral vectors expressing the individual components, or viral vectors co-expressing both components.

As used, herein, a “vaccine” is a formulation that, when administered to an animal subject such as a mammal (e.g. a human or other primate) stimulates or provides a protective immune response against Plasmodium parasitic infection. The immune response may be a humoral and/or cell-mediated immune response. A vaccine of the invention can be used, for example, to protect a subject from the effects of P. falciparum infection (i.e. malaria).

Thus, the composition comprising an antigen (e.g., RIPR antigen), fusion protein (e.g., RIPR-CyRPA fusion), antibody or vector (e.g., a viral vector, RNA vaccine or DNA plasmid) of the invention typically provide a highly effective cross-strain GIA against the Plasmodium parasite. Thus, in one embodiment, the invention provides protection (such as long term protection) against disease caused by Plasmodium parasites. Typically, a composition comprising an antigen (e.g., RIPR antigen), fusion protein (e.g., RIPR-CyRPA fusion), antibody or vector (e.g., a viral vector, RNA vaccine or DNA plasmid) of the invention provides an antibody response (e.g. a neutralising antibody response) to Plasmodium parasitic infection. The composition comprising an antigen (e.g., RIPR antigen), fusion protein (e.g., RIPR-CyRPA fusion), antibody or vector (e.g., a viral vector, RNA vaccine or DNA plasmid) of the invention may be used to confer pre-erythrocytic or transmission-blocking protection against Plasmodium parasites.

The treatment and/or prevention of malaria according to the present invention may further comprise boosting a subject. Such “boosting” may comprise the administration of a pox virus, such as MVA.

### Pharmaceutical Compositions and Formulations

The term “vaccine” is herein used interchangeably with the terms “therapeutic/prophylactic composition”, “formulation” or “medicament”.

The vaccine of the invention (as defined above) can be combined or administered in addition to a pharmaceutically acceptable carrier. Alternatively or in addition the vaccine of the invention can further be combined with one or more of a salt, excipient, diluent, adjuvant, immunoregulatory agent and/or antimicrobial compound.

Pharmaceutically acceptable salts include acid addition salts formed with inorganic acids such as, for example, hydrochloric or phosphoric acids, or with organic acids such as acetic, oxalic, tartaric, maleic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Administration of immunogenic compositions, therapeutic formulations, medicaments and prophylactic formulations (e.g. vaccines) is generally by conventional routes e.g. intravenous, subcutaneous, intraperitoneal, or mucosal routes. The administration may be by parenteral injection, for example, a subcutaneous, intradermal or intramuscular injection. Formulations comprising antibodies may be particularly suited to administration intravenously, intramuscularly, intradermally, or subcutaneously.

Accordingly, immunogenic compositions, therapeutic formulations, medicaments and prophylactic formulations (e.g. vaccines) of the invention are typically prepared as injectables, either as liquid solutions or suspensions. Solid forms suitable for solution in, or suspension in, liquid prior to injection may alternatively be prepared. The preparation may also be emulsified, or the peptide encapsulated in liposomes or microcapsules.

The active immunogenic ingredients (such as the antigens, antibodies, or binding fragments thereof, fusion proteins, viral vectors, RNA vaccines or DNA plasmids) are often mixed with excipients which are pharmaceutically acceptable and compatible with the active ingredient. Suitable excipients are, for example, water, saline, dextrose, glycerol, ethanol, or the like and combinations thereof. In addition, if desired, the vaccine may contain minor amounts of auxiliary substances such as wetting or emulsifying agents, pH buffering agents, and/or adjuvants which enhance the effectiveness of the vaccine.

Generally, the carrier is a pharmaceutically-acceptable carrier. Non-limiting examples of pharmaceutically acceptable carriers include water, saline, and phosphate-buffered saline. In some embodiments, however, the composition is in lyophilized form, in which case it may include a stabilizer, such as BSA. In some embodiments, it may be desirable to formulate the composition with a preservative, such as thiomersal or sodium azide, to facilitate long term storage.

Examples of additional adjuvants which may be effective include but are not limited to: complete Freund's adjuvant (CFA), Incomplete Freund's adjuvant (IFA), Saponin, a purified extract fraction of Saponin such as Quil A, a derivative of Saponin such as QS-21, lipid particles based on Saponin such as ISCOM/ISCOMATRIX/Matrix-M™, E. coli heat labile toxin (LT) mutants such as LTK63 and/or LTK72, aluminium hydroxide, N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryl oxy)-ethylamine (CGP 19835A, referred to as MTP-PE), and RIBI, which contains three components extracted from bacteria, monophosphoryl lipid A, trehalose dimycolate and cell wall skeleton (MPL+TDM+CWS) in a 2% squalene/Tween 80 emulsion, the MF59 formulation developed by Novartis, and the AS02, AS01, AS03 and AS04 adjuvant formulations developed by GSK Biologicals (Rixensart, Belgium).

Examples of buffering agents include, but are not limited to, sodium succinate (pH 6.5), and phosphate buffered saline (PBS; pH 6.5 and 7.5).

Additional formulations which are suitable for other modes of administration include suppositories and, in some cases, oral formulations or formulations suitable for distribution as aerosols. For suppositories, traditional binders and carriers may include, for example, polyalkylene glycols or triglycerides; such suppositories may be formed from mixtures containing the active ingredient in the range of 0.5% to 10%, preferably 1%-2%.

Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders.

It is within the routine practice of a clinician to determine an effective amount of a vaccine composition of the invention. An effective amount is an amount sufficient to elicit a protective immune response against malaria. A clinician will also be able to determine appropriate dosage interval using routine skill.

### Sequence Homology

An amino acid modification according to the invention may be a substitution, deletion, addition or other modification, including post-translational modification, unless the relevant disclosure explicitly says otherwise. Preferably said modifications are amino acid substitutions. In other words, the amino acid at a specified position within the antigen of the invention is substituted by a naturally occurring or non-naturally occurring amino acid that is different to the amino acid present at that position in the sequence from which the antigen of the invention is derived. Alternatively, the amino acid at a specified position within the antigen of the invention may be modified post-translationally. Post-translational modifications include glycosylations, acetylations, acylations, de-aminations, phosphorylisations, isoprenylisations, glycosyl phosphatidyl inositolisations and further modifications known to a person skilled in the art.

The modification of one or more amino acid position as described herein may be performed, for example, by specific mutagenesis, or any other method known in the art.

In embodiments in which one or more amino acid position is substituted relative to the corresponding antigen from which the antigen of the invention is derived, the substitution may be a conservative substitution or a non-conservative substitution. A conservative substitution is defined as substitution by an amino acid pertaining to the same physiochemical group to the amino acid present in the antigen from which the antigen of the invention is derived. A non-conservative amino acid substitution is defined as substitution by an amino acid pertaining to a different physiochemical group to the amino acid present in the antigen from which the antigen of the invention is derived. In more detail, amino acids are, in principle, divided into different physiochemical groups. Aspartate and glutamate belong to the negatively-charged amino acids. Histidine, arginine and lysine belong to the positively-charged amino acids. Asparagine, glutamine, serine, threonine, cysteine and tyrosine belong to the polar amino acids. Glycine, alanine, valine, leucine, isoleucine, methionine, proline, phenylalanine and tryptophan belong to the non-polar amino acids. Aromatic side groups are to be found among the amino acids histidine, phenylalanine, tyrosine and tryptophan. Thus, as a non-limiting example, a conservative substation may involve the substitution of a non-polar amino acid by another non-polar amino acid, such as substituting leucine with isoleucine. As another non-limiting example, a non-conservative substitution may involve the substation of a non-polar amino acid (e.g. leucine) with a negatively-charged amino acid (e.g. aspartate), a positively-charged amino acid (e.g. arginine), or a polar amino acid (e.g. asparagine).

Conventional methods for determining amino acid sequence identity are known in the art. The terms “sequence identity” and “sequence homology” are considered synonymous in this specification.

By way of example, a polypeptide of interest may comprise an amino acid sequence having at least 70, 75, 80, 82, 84, 86, 88, 90, 92, 94, 96, 98, 99 or 100% amino acid sequence identity with the amino acid sequence of a reference polypeptide.

There are many established algorithms available to align two amino acid sequences. Typically, one sequence acts as a reference sequence, to which test sequences may be compared. The sequence comparison algorithm calculates the percentage sequence identity for the test sequence(s) relative to the reference sequence, based on the designated program parameters. Alignment of amino acid sequences for comparison may be conducted, for example, by computer implemented algorithms (e.g. GAP, BESTFIT, FASTA or TFASTA), or BLAST and BLAST 2.0 algorithms.

The BLOSUM62 table shown below is an amino acid substitution matrix derived from about 2,000 local multiple alignments of protein sequence segments, representing highly conserved regions of more than 500 groups of related proteins (Henikoff & Henikoff, Proc. Natl. Acad. Sci. USA 89:10915-10919, 1992; incorporated herein by reference). Amino acids are indicated by the standard one-letter codes. The percent identity is calculated as:

$$\frac{Total number of identical matches}{[ length of the longer sequence plus the number of gaps} \times 100$$

Introduced into the longer sequence in order to align the two sequences]

BLOSUM62 table

A R N D C Q E G H I L K M F P S T W Y V
A 4
R −1 5
N −2 0 6
D −2 −2 1 6
C 0−3 −3 −3 9
Q −1 1 0 0 −3 5
E −1 0 0 2 −4 2 5
G 0 −2 0 −1 −3 −2 −2 6
H −2 0 1 −1 −3 0 0 −2 8
I −1 −3 −3 −3 −1 −3 −3 −4 −3 4
L −1 −2 −3 −4 −1 −2 −3 −4 −3 2 4
K −1 2 0 −1 −3 1 1 −2 −1 −3 −2 5
M −1 −1 −2 −3 −1 0 −2 −3 −2 1 2 −1 5
F −2 −3 −3 −3 −2 −3 −3 −3 −1 0 0 −3 0 6
P −1 −2 −2 −1 −3 −1 −1 −2 −2 −3 −3 −1 −2 −4 7
S 1 −1 1 0 −1 0 0 0 −1 −2 −2 0 −1 −2 −1 4
T 0 −1 0 −1 −1 −1 −1 −2 −2 −1 −1 −1 −1 −2 −1 1 5
W −3 −3 −4 −4 −2 −2 −3 −2 −2 −3 −2 −3 −1 1 −4 −3 −2 1 1
Y −2 −2 −2 −3 −2 −1 −2 −3 2 −1 −1 −2 −1 3 −3 −2 −2 2 7
V 0 −3 −3 −3 −1 −2 −2 −3 −3 3 1 −2 1 −1 −2 −2 0 −3 −1 4

In a homology comparison, the identity may exist over a region of the sequences that is at least 10 amino acid residues in length (e.g. at least 15, 20, 30, 40, 50, 75, 100, 150, 200, 250, 300, 350, 400, 450, 500 or 520 amino acid residues in length)—e.g. up to the entire length of the reference sequence.

Substantially homologous polypeptides have one or more amino acid substitutions, deletions, or additions. In many embodiments, those changes are of a minor nature, for example, involving only conservative amino acid substitutions. Conservative substitutions are those made by replacing one amino acid with another amino acid within the following groups: Basic: arginine, lysine, histidine; Acidic: glutamic acid, aspartic acid; Polar: glutamine, asparagine; Hydrophobic: leucine, isoleucine, valine; Aromatic: phenylalanine, tryptophan, tyrosine; Small: glycine, alanine, serine, threonine, methionine. Substantially homologous polypeptides also encompass those comprising other substitutions that do not significantly affect the folding or activity of the polypeptide; small deletions, typically of 1 to about 30 amino acids (such as 1-10, or 1-5 amino acids); and small amino- or carboxyl-terminal extensions, such as an amino-terminal methionine residue, a small linker peptide of up to about 20-25 residues, or an affinity tag.

### Sequence Information

SEQ ID NO: 1: Full length RIPR (Genbank XP_001351305)
SEQ ID NO: 2: RIPR EGF domains 5-8, corresponding to P716 to D900 of SEQ ID NO: 1
SEQ ID NO: 3: RIPR EGF domains 7-8, corresponding to D817 to V897 of SEQ ID NO: 1
SEQ ID NO: 4: RIPR C-terminal domain (CTD)
SEQ ID NO: 5: RIPR EGF domains 5-8 with RIPR CTD
SEQ ID NO: 6: RIPR EGF domains 7-8 with RIPR CTD
SEQ ID NO: 7: Exemplary CyRPA sequence
SEQ ID NO: 8: SPYTAG sequence
SEQ ID NO: 9: SPYTAG with additional linker sequences
SEQ ID NO: 10: Fusion protein comprising RIPR EGF domains 5-8 and CyRPA
SEQ ID NO: 11: Fusion protein comprising RIPR EGF domains 7-8 and CyRPA
SEQ ID NO: 12: R58C
SEQ ID NO: 13: R78C
SEQ ID NO: 14: Variant RIPR EGF domains 5-8 and a RIPR CTD
SEQ ID NO: 15: Variant fusion protein comprising RIPR EGF domains 5-8, a RIPR CTD and CyRPA
SEQ ID NO: 16: R58CC, Variant fusion protein comprising RIPR EGF domains 5-8, a RIPR CTD and CyRPA
SEQ ID NO: 17: Variant RIPR EGF domains 7-8 and a RIPR CTD
SEQ ID NO: 18: Variant fusion protein comprising RIPR EGF domains 7-8, a RIPR CTD and CyRPA
SEQ ID NO: 19: R78CC, Variant fusion protein comprising RIPR EGF domains 7-8, a RIPR CTD and CyRPA
SEQ ID NO: 20: Full length PfRH5 amino acid sequence (3D7) including signal sequence SEQ ID NO: 21: Full length PfRH5 amino acid sequence (7G8) including signal sequence
SEQ ID NO: 22: PfRH5 amino acid sequence (3D7) excluding signal sequence and flexible N-terminal region (amino acids 1 to 139) (3D7 RH5AN)
SEQ ID NO: 23: PfRH5 amino acid sequence (7G8) excluding signal sequence and flexible N-terminal region (amino acids 1 to 139): (7G8 RH5AN)
SEQ ID NO: 24: PfRH5 amino acid sequence (3D7) excluding signal sequence and flexible N-terminal region (amino acids 1 to 159)
SEQ ID NO: 25: PfRH5 amino acid sequence (7G8) excluding signal sequence and flexible N-terminal region (amino acids 1 to 159)
SEQ ID NO: 26: PfRH5 amino acid sequence (3D7) excluding signal sequence, flexible N-terminal (amino acids 1 to 139) and flexible loop (amino acids 248 to 296) regions (3D7 RH5ANL)
SEQ ID NO: 27: PfRH5 amino acid sequence (7G8) excluding signal sequence, flexible N-terminal (amino acids 1 to 139) and flexible loop (amino acids 248 to 296) regions: (7G8 RH5ANL)
SEQ ID NO: 28: PfRH5 amino acid sequence (3D7) excluding signal sequence, flexible N-terminal (amino acids 1 to 159) and flexible loop (amino acids 248 to 296) regions
SEQ ID NO: 29: PfRH5 amino acid sequence (7G8) excluding signal sequence, flexible N-terminal (amino acids 1 to 159) and flexible loop (amino acids 248 to 296) regions
SEQ ID NO: 30: RH5.1
SEQ ID NO: 31: Full length RH5.1
SEQ ID NO: 32: PfRH5 (PfRH5FL)
SEQ ID NO: 33: PfRH5ANL

SEQ ID NO: 1: Full length RIPR (Genbank XP_001351305), RIPR EGF domains 5-8 are
shown in bold

| | |
|---|---|
| 1 | MFRIFFTLLI IILIKKTSAI DLIEGIFYEK NEIDKLTFSL DHRVRDNLKT DLILNNNGEN |
| 61 | DYAYLNKYVY TILNRDSTEK IKTFFSHNKD MKSCDYFISK EYNSSDKTNQ ICYKKTFCGV |
| 121 | VIPNSEEIKT NKITNDKLYC AHFNSTHIII YYISQPLLLE PHVVYEETFF EKGKNDQINC |
| 181 | QGMYISLRSV HVHTHNAILQ QETLTYIKNL CDGKNNCKFD FDSIKYENKS LTHYLFFINI |
| 241 | QYQCISPLNL QENEMCDVYN DDTHKATCKY GFNKIELLKN VCEENYRCTQ DICSVNQFCD |
| 301 | GENETCTCKT SLLPSAKNNC EYNDLCTVLN CPENSTCEQI GNGKKAECKC ENGKYYHNNK |
| 361 | CYTKNDLELA IKIEPHKKEK FYKNNLYQGK ALKPEYIFMQ CENGFSIEVI NAYVSCYRVS |
| 421 | FNLNKLKYVT ESLKKMCDGK TKCAYGNTID PIDDLNHHNI CNNFNTIFKY DYLCVFNNQN |
| 481 | ITSDKNSHLH SNIPSLYNSS ILPDINKSKF HLISRNSRTN QYPHNNISML EIQNEISSHN |
| 541 | SNQFSTDPHT NSNNINNMNI KKVEIFRSRF SSKLQCQGGK INIDKAILKG GEGCNDLLLT |
| 601 | NSLKSYCNDL SECDIGLIYH FDTYCINDQY LFVSYSCSNL CNKCHNNSTC YGNRFNYDCF |
| 661 | CDNPYISKYG NKLCERPNDC ESVLCSQNQV CQILPNDKLI CQCEEGYKNV KGKCVPDNKC |
| 721 | DLSCPSNKVC VIENGKQTCK CSERFVLENG VCICANDYKM EDGINCIAKN KCKRKEYENI |
| 781 | CTNPNEMCAY NEETDIVKCE CKEHYYRSSR GECILNDYCK DINCKENEEC SIVNFKPECV |
| 841 | CKENLKKNNK GECIYENSCL INEGNCPKDS KCIYREYKPH ECVCNKQGHV AVNGKCVLED |
| 901 | KCVHNKKCSE NSICVNVMNK EPICVCTYNY YKKDGVCLIQ NPCLKDNGGC SRNSECTFKY |
| 961 | SKINCTCKEN YKNKDDSCVP NTNEYDESFT FQYNDDASII LGACGMIEFS YIYNQIIWKI |
| 1021 | NNSKESYVFY YDYPTAGNIE VQIKNEIFHT IIYLKKKIGN SVIYDDFQVD HQTCIYENVF |
| 1081 | YYSNQN |

SEQ ID NO: 2: RIPR EGF domains 5-8, P716 to D900 of SEQ ID NO: 1
PDNKCDLSCPSNKVCVIENGKQTCKCSERFVLENGVCICANDYKMEDGINCIAKNKCKRKEYENICTNPNEMCAYN
EETDIVKCECKEHYYRSSRGECILNDYCKDINCKENEECSIVNFKPECVCKENLKKNNKGECIYENSCLINEGNCPKDS
KCIYREYKPHECVCNKQGHVAVNGKCVLED
SEQ ID NO: 3: RIPR EGF domains 7-8, D817 to V897 of SEQ ID NO: 1
DYCKDINCKENEECSIVNFKPECVCKENLKKNNKGECIYENSCLINEGNCPKDSKCIYREYKPHECVCNKQGHVAVNG
KCV
SEQ ID NO: 4: RIPR C-terminal domain (CTD)
PNTNEYDESFTFQYNDDASIILGACGMIEFSYIYNQIIWKIQNSKESYVFYYDYPTAGNIEVQIKNEIFHTIIY
LKKKIGNSVIYDDFQVDHQTCIYENVFYYSNQN
SEQ ID NO: 5: RIPR EGF domains 5-8 with RIPR CTD, Underlined = RIPR EGF
domains 5-8, Italics = CTD
PDNKCDLSCPSNKVCVIENGKQTCKCSERFVLENGVCICANDYKMEDGINCIAKNKCKRKEYENICTNPNEMCAYN
EETDIVKCECKEHYYRSSRGECILNDYCKDINCKENEECSIVNFKPECVCKENLKKNNKGECIYENSCLINEGNCPKDS
KCIYREYKPHECVCNKQGHVAVNGKCVLEDGGGGTPNTNEYDESFTFQYNDDASIILGACGMIEFSYIYNQIIWKIQ
NSKESYVFYYDYPTAGNIEVQIKNEIFHTIIYLKKKIGNSVIYDDFQVDHQTCIYENVFYYSNQN
SEQ ID NO: 6: RIPR EGF domains 7-8 with RIPR CTD, Underlined = RIPR EGF
domains 7-8, Italics = CTD
DYCKDINCKENEECSIVNFKPECVCKENLKKNNKGECIYENSCLINEGNCPKDSKCIYREYKPHECVCNKQGHVAVN
GKCVGGGTPNTNEYDESFTFQYNDDASIILGACGMIEFSYIYNQIIWKIQNSKESYVFYYDYPTAGNIEVQIKNEIFHT
IIYLKKKIGNSVIYDDFQVDHQTCIYENVFYYSNQN
SEQ ID NO: 7: CyRPA (334 amino acids)
DSRHVFIRTELSFIKNNVPCIRDMFFIYKRELYNICLDDLKGEEDETHIYVQKKVKDSWITLNDLFKETDLTGRPHIFAY
VDVEEIIILLCEDEEFSNRKKDMTCHRFYSNDGKEYNNAEITISDYILKDKLLSSYVSLPLKIENREYFLICGVSPYKFKDD
NKKDDILCMASHDKGETWGTKIVIKYDNYKLGVQYFFLRPYISKNDLSFHFYVGDNINNVKNVNFIECTHEKDLEFV
CSNRDFLKDNKVLQDVSTLNDEYIVSYGNDNNFAECYIFFNNENSILIKPEKYGNTAAGCYGGTFVKIDENRALFIYSS
SQGIYNIHTIYYANYE
SEQ ID NO: 8 SPYTAG
AHIVMVDAYKPTK
SEQ ID NO: 9: SPYTAG with linkers
GGGGSGGGGSAHIVMVDAYKPTKGGGGSGGGGS
SEQ ID NO: 10: R58C, without signal sequence or purification tag, Underlined = RIPR
component, Bold = CyRPA component
PDNKCDLSCPSNKVCVIENGKQTCKCSERFVLENGVCICANDYKMEDGINCIAKNKCKRKEYENICTNPNEMCAYN
EETDIVKCECKEHYYRSSRGECILNDYCKDINCKENEECSIVNFKPECVCKENLKKNNKGECIYENSCLINEGNCPKDS
KCIYREYKPHECVCNKQGHVAVNGKCVLEDGGGGSGGGGSAHIVMVDAYKPTKGGGGSGGGGSDSRHVFIRTEL
SFIKNNVPCIRDMFFIYKRELYNICLDDLKGEEDETHIYVQKKVKDSWITLNDLFKETDLTGRPHIFAYVDVEEIIILL
CEDEEFSNRKKDMTCHRFYSNDGKEYNNAEITISDYILKDKLLSSYVSLPLKIENREYFLICGVSPYKFKDDNKKDDI
LCMASHDKGETWGTKIVIKYDNYKLGVQYFFLRPYISKNDLSFHFYVGDNINNVKNVNFIECTHEKDLEFVCSNR
DFLKDNKVLQDVSTLNDEYIVSYGNDNNFAECYIFFNNENSILIKPEKYGNTAAGCYGGTFVKIDENRALFIYSSSQ
GIYNIHTIYYANYE
SEQ ID NO: 11: R78C, without signal sequence or purification tag, Underlined = RIPR
component, Bold = CyRPA component
DYCKDINCKENEECSIVNFKPECVCKENLKKNNKGECIYENSCLINEGNCPKDSKCIYREYKPHECVCNKQGHVAVN
GKCVGGGGSGGGGSAHIVMVDAYKPTKGGGGSGGGGSDSRHVFIRTELSFIKNNVPCIRDMFFIYKRELYNICLD
DLKGEEDETHIYVQKKVKDSWITLNDLFKETDLTGRPHIFAYVDVEEIIILLCEDEEFSNRKKDMTCHRFYSNDGKE
YNNAEITISDYILKDKLLSSYVSLPLKIENREYFLICGVSPYKFKDDNKKDDILCMASHDKGETWGTKIVIKYDNYKL
GVQYFFLRPYISKNDLSFHFYVGDNINNVKNVNFIECTHEKDLEFVCSNRDFLKDNKVLQDVSTLNDEYIVSYGN
DNNFAECYIFFNNENSILIKPEKYGNTAAGCYGGTFVKIDENRALFIYSSSQGIYNIHTIYYANYE
SEQ ID NO: 12: Full length R58C, Underlined = RIPR component, Bold = CyRPA
component
MGWSCIILFLVATATGVHSPDNKCDLSCPSNKVCVIENGKQTCKCSERFVLENGVCICANDYKMEDGINCIAKNKC
KRKEYENICTNPNEMCAYNEETDIVKCECKEHYYRSSRGECILNDYCKDINCKENEECSIVNFKPECVCKENLKKNNK
GECIYENSCLINEGNCPKDSKCIYREYKPHECVCNKQGHVAVNGKCVLEDGGGGSGGGGSAHIVMVDAYKPTKGG
GGSGGGGSDSRHVFIRTELSFIKNNVPCIRDMFFIYKRELYNICLDDLKGEEDETHIYVQKKVKDSWITLNDLFKET
DLTGRPHIFAYVDVEEIIILLCEDEEFSNRKKDMTCHRFYSNDGKEYNNAEITISDYILKDKLLSSYVSLPLKIENREYF
LICGVSPYKFKDDNKKDDILCMASHDKGETWGTKIVIKYDNYKLGVQYFFLRPYISKNDLSFHFYVGDNINNVKN
VNFIECTHEKDLEFVCSNRDFLKDNKVLQDVSTLNDEYIVSYGNDNNFAECYIFFNNENSILIKPEKYGNTAAGCY
GGTFVKIDENRALFIYSSSQGIYNIHTIYYANYEGGGGSEPEA
SEQ ID NO: 13: Full length R78C, Underlined = RIPR component, Bold = CyRPA
component
MGWSCIILFLVATATGVHSDYCKDINCKENEECSIVNFKPECVCKENLKKNNKGECIYENSCLINEGNCPKDSKCIYR
EYKPHECVCNKQGHVAVNGKCVGGGGSGGGGSAHIVMVDAYKPTKGGGGSGGGGSDSRHVFIRTELSFIKNNV
PCIRDMFFIYKRELYNICLDDLKGEEDETHIYVQKKVKDSWITLNDLFKETDLTGRPHIFAYVDVEEIIILLCEDEEFS
NRKKDMTCHRFYSNDGKEYNNAEITISDYILKDKLLSSYVSLPLKIENREYFLICGVSPYKFKDDNKKDDILCMASH
DKGETWGTKIVIKYDNYKLGVQYFFLRPYISKNDLSFHFYVGDNINNVKNVNFIECTHEKDLEFVCSNRDFLKDN
KVLQDVSTLNDEYIVSYGNDNNFAECYIFFNNENSILIKPEKYGNTAAGCYGGTFVKIDENRALFIYSSSQGIYNIH
TIYYANYEGGGGSEPEA
SEQ ID NO: 14: R58 + CTD + Linker, Underlined = EGF domains 5-8, Italics = RIPR
CTD, Italics underlined = linker
PDNKCDLSCPSNKVCVIENGKQTCKCSERFVLENGVCICANDYKMEDGINCIAKNKCKRKEYENICTNPNEMCAYN
EETDIVKCECKEHYYRSSRGECILNDYCKDINCKENEECSIVNFKPECVCKENLKKNNKGECIYENSCLINEGNCPKDS
KCIYREYKPHECVCNKQGHVAVNGKCVLEDGGGGTPNTNEYDESFTFQYNDDASIILGACGMIEFSYIYNQIIWKIQ
NSKESYVFYYDYPTAGNIEVQIKNEIFHTIIYLKKKIGNSVIYDDFQVDHQTCIYENVFYYSNQNGGGGSGGGGSAHI
VMVDAYKPTKGGGGSGGGGS
SEQ ID NO: 15: R58CC, without signal sequence or purification tag, Underlined = EGF
domains 5-8, Italics = RIPR CTD, Italics underlined = linker, Bold = CyRPA
PDNKCDLSCPSNKVCVIENGKQTCKCSERFVLENGVCICANDYKMEDGINCIAKNKCKRKEYENICTNPNEMCAYN
EETDIVKCECKEHYYRSSRGECILNDYCKDINCKENEECSIVNFKPECVCKENLKKNNKGECIYENSCLINEGNCPKDS
KCIYREYKPHECVCNKQGHVAVNGKCVLEDGGGGTPNTNEYDESFTFQYNDDASIILGACGMIEFSYIYNQIIWKIQ
NSKESYVFYYDYPTAGNIEVQIKNEIFHTIIYLKKKIGNSVIYDDFQVDHQTCIYENVFYYSNQNGGGGSGGGGSAHI
VMVDAYKPTKGGGGSGGGGSDSRHVFIRTELSFIKNNVPCIRDMFFIYKRELYNICLDDLKGEEDETHIYVQKKVK
DSWITLNDLFKETDLTGRPHIFAYVDVEEIIILLCEDEEFSNRKKDMTCHRFYSNDGKEYNNAEITISDYILKDKLLSS
YVSLPLKIENREYFLICGVSPYKFKDDNKKDDILCMASHDKGETWGTKIVIKYDNYKLGVQYFFLRPYISKNDLSFH
FYVGDNINNVKNVNFIECTHEKDLEFVCSNRDFLKDNKVLQDVSTLNDEYIVSYGNDNNFAECYIFFNNENSILIK
PEKYGNTAAGCYGGTFVKIDENRALFIYSSSQGIYNIHTIYYANYE
SEQ ID NO: 16: Full length R58CC, Underlined = EGF domains 5-8, Italics = RIPR CTD,
Italics underlined = linker, Bold = CyRPA
MGWSCIILFLVATATGVHSGTPDNKCDLSCPSNKVCVIENGKQTCKCSERFVLENGVCICANDYKMEDGINCIAKN
KCKRKEYENICTNPNEMCAYNEETDIVKCECKEHYYRSSRGECILNDYCKDINCKENEECSIVNFKPECVCKENLKKN
NKGECIYENSCLINEGNCPKDSKCIYREYKPHECVCNKQGHVAVNGKCVLEDGGGGTPNTNEYDESFTFQYNDDAS
IILGACGMIEFSYIYNQIIWKIQNSKESYVFYYDYPTAGNIEVQIKNEIFHTIIYLKKKIGNSVIYDDFQVDHQTCIYENVF
YYSNQNGGGGSGGGGSAHIVMVDAYKPTKGGGGSGGGGSDSRHVFIRTELSFIKNNVPCIRDMFFIYKRELYNIC
LDDLKGEEDETHIYVQKKVKDSWITLNDLFKETDLTGRPHIFAYVDVEEIIILLCEDEEFSNRKKDMTCHRFYSNDG
KEYNNAEITISDYILKDKLLSSYVSLPLKIENREYFLICGVSPYKFKDDNKKDDILCMASHDKGETWGTKIVIKYDNY
KLGVQYFFLRPYISKNDLSFHFYVGDNINNVKNVNFIECTHEKDLEFVCSNRDFLKDNKVLQDVSTLNDEYIVSYG
NDNNFAECYIFFNNENSILIKPEKYGNTAAGCYGGTFVKIDENRALFIYSSSQGIYNIHTIYYANYEGGGGSEPEA
SEQ ID NO: 17: R78 + CTD + Linker, Underlined = EGF domains 7-8, Italics = RIPR
CTD, Italics underlined = linker
DYCKDINCKENEECSIVNFKPECVCKENLKKNNKGECIYENSCLINEGNCPKDSKCIYREYKPHECVCNKQGHVAVN
GKCVGGGTPNTNEYDESFTFQYNDDASIILGACGMIEFSYIYNQIIWKIQNSKESYVFYYDYPTAGNIEVQIKNEIFHT
IIYLKKKIGNSVIYDDFQVDHQTCIYENVFYYSNQNGGGGSGGGGSAHIVMVDAYKPTKGGGGSGGGGS
SEQ ID NO: 18: R78CC, without signal sequence or purification tag, Underlined = EGF
domains 7-8, Italics = RIPR CTD, Italics underlined = linker, Bold = CyRPA
DYCKDINCKENEECSIVNFKPECVCKENLKKNNKGECIYENSCLINEGNCPKDSKCIYREYKPHECVCNKQGHVAVN
GKCVGGGTPNTNEYDESFTFQYNDDASIILGACGMIEFSYIYNQIIWKIQNSKESYVFYYDYPTAGNIEVQIKNEIFHT
IIYLKKKIGNSVIYDDFQVDHQTCIYENVFYYSNQNGGGGSGGGGSAHIVMVDAYKPTKGGGGSGGGGSDSRHVF
IRTELSFIKNNVPCIRDMFFIYKRELYNICLDDLKGEEDETHIYVQKKVKDSWITLNDLFKETDLTGRPHIFAYVDVE
EIIILLCEDEEFSNRKKDMTCHRFYSNDGKEYNNAEITISDYILKDKLLSSYVSLPLKIENREYFLICGVSPYKFKDDNK
KDDILCMASHDKGETWGTKIVIKYDNYKLGVQYFFLRPYISKNDLSFHFYVGDNINNVKNVNFIECTHEKDLEFV
CSNRDFLKDNKVLQDVSTLNDEYIVSYGNDNNFAECYIFFNNENSILIKPEKYGNTAAGCYGGTFVKIDENRALFI
YSSSQGIYNIHTIYYANYE
SEQ ID NO: 19: Full length R78CC, Underlined = EGF domains 7-8, Italics = RIPR CTD,
Italics underlined = linker, Bold = CyRPA
MGWSCIILFLVATATGVHSDYCKDINCKENEECSIVNFKPECVCKENLKKNNKGECIYENSCLINEGNCPKDSKCIYR
EYKPHECVCNKQGHVAVNGKCVGGGTPNTNEYDESFTFQYNDDASIILGACGMIEFSYIYNQIIWKIQNSKESYVFY
YDYPTAGNIEVQIKNEIFHTIIYLKKKIGNSVIYDDFQVDHQTCIYENVFYYSNQNGGGGSGGGGSAHIVMVDAYKP
TKGGGGSGGGGSDSRHVFIRTELSFIKNNVPCIRDMFFIYKRELYNICLDDLKGEEDETHIYVQKKVKDSWITLNDL
FKETDLTGRPHIFAYVDVEEIIILLCEDEEFSNRKKDMTCHRFYSNDGKEYNNAEITISDYILKDKLLSSYVSLPLKIEN
REYFLICGVSPYKFKDDNKKDDILCMASHDKGETWGTKIVIKYDNYKLGVQYFFLRPYISKNDLSFHFYVGDNINN
VKNVNFIECTHEKDLEFVCSNRDFLKDNKVLQDVSTLNDEYIVSYGNDNNFAECYIFFNNENSILIKPEKYGNTAA
GCYGGTFVKIDENRALFIYSSSQGIYNIHTIYYANYEGGGGSEPEA
SEQ ID NO: 20: Full length PfRH5 amino acid sequence (3D7) including signal sequence

| | |
|---|---|
| 1 | MIRIKKKLIL TIIYIHLFIL NRLSFENAIK KTKNQENNLT LLPIKSTEEE KDDIKNGKDI |
| 61 | KKEIDNDKEN IKTNNAKDHS TYIKSYLNTN VNDGLKYLFI PSHNSFIKKY SVFNQINDGM |
| 121 | LLNEKNDVKN NEDYKNVDYK NVNFLQYHFK ELSNYNIANS IDILQEKEGH LDFVIIPHYT |
| 181 | FLDYYKHLSY NSIYHKSSTY GKCIAVDAFI KKINETYDKV KSKCNDIKND LIATIKKLEH |
| 241 | PYDINNKNDD SYRYDISEEI DDKSEETDDE TEEVEDSIQD TDSNHTPSNK KKNDLMNRTF |
| 301 | KKMMDEYNTK KKKLIKCIKN HENDFNKICM DMKNYGTNLF EQLSCYNNNF CNTNGIRYHY |
| 361 | DEYIHKLILS VKSKNLNKDL SDMTNILQQS ELLLTNLNKK MGSYIYIDTI KFIHKEMKHI |
| 421 | FNRIEYHTKI INDKTKIIQD KIKLNIWRTF QKDELLKRIL DMSNEYSLFI TSDHLRQMLY |
| 481 | NTFYSKEKHL NNIFHHLIYV LQMKFNDVPI KMEYFQTYKK NKPLTQ |

SEQ ID NO: 21 Full length PfRH5 amino acid sequence (7G8) including signal sequence

| | |
|---|---|
| 1 | MIRIKKKLIL TIIYIHLFIL NRLSFENAIK KTKNQENNLT LLPIKSTEEE KDDIKNGKDI |
| 61 | KKEIDNDKEN IKTNNAKDHS TYIKSYLNTN VNDGLKYLFI PSHNSFIKKY SVFNQINDGM |
| 121 | LLNEKNDVKN NEDYKNVDYK NVNFLQYHFK ELSNYNIANS IDILQEKEGH LDFVIIPHYT |
| 181 | FLDYYKHLSY NSIYHKSSTY GKYIAVDAFI KKINETYDKV KSKCNDIKND LIATIKKLEH |
| 241 | PYDINNKNDD SYRYDISEEI DDKSEETDDE TEEVEDSIQD TDSNHTPSNK KKNDLMNRTF |
| 301 | KKMMDEYNTK KKKLIKCIKN HENDFNKICM DMKNYGTNLF EQLSCYNNNF CNTNGIRYHY |
| 361 | DEYIHKLILS VKSKNLNKDL SDMTNILQQS ELLLTNLNKK MGSYIYIDTI KFIHKEMKHI |
| 421 | FNRIEYHTKI INDKTKIIQD KIKLNIWRTF QKDELLKRIL DMSNEYSLFI TSDHLRQMLY |
| 481 | NTFYSKEKHL NNIFHHLIYV LQMKFNDVPI KMEYFQTYKK NKPLTQ |

SEQ ID NO: 22: PfRH5 amino acid sequence (3D7) excluding signal sequence and flexible
N-terminal region (amino acids 1 to 139) (3D7 RH5ΔN)

| | |
|---|---|
| 1 | KNVNFLQYHF KELSNYNIAN SIDILQEKEG HLDFVIIPHY TFLDYYKHLS YNSIYHKSST |
| 61 | YGKCIAVDAF IKKINETYDK VKSKCNDIKN DLIATIKKLE HPYDINNKND DSYRYDISEE |
| 121 | IDDKSEETDD ETEEVEDSIQ DTDSNHTPSN KKKNDLMNRT FKKMMDEYNT KKKKLIKCIK |
| 181 | NHENDFNKIC MDMKNYGTNL FEQLSCYNNN FCNTNGIRYH YDEYIHKLIL SVKSKNLNKD |
| 241 | LSDMTNILQQ SELLLTNLNK KMGSYIYIDT IKFIHKEMKH IFNRIEYHTK IINDKTKIIQ |
| 301 | DKIKLNIWRT FQKDELLKRI LDMSNEYSLF ITSDHLRQML YNTFYSKEKH LNNIFHHLIY |
| 361 | VLQMKFNDVP IKMEYFQTYK KNKPLTQ |

SEQ ID NO: 23: PfRH5 amino acid sequence (7G8) excluding signal sequence and flexible
N-terminal region (amino acids 1 to 139): (7G8 RH5ΔN)

| | |
|---|---|
| 1 | KNVNFLQYHF KELSNYNIAN SIDILQEKEG HLDFVIIPHY TFLDYYKHLS YNSIYHKSST |
| 61 | YGKYIAVDAF IKKINETYDK VKSKCNDIKN DLIATIKKLE HPYDINNKND DSYRYDISEE |
| 121 | IDDKSEETDD ETEEVEDSIQ DTDSNHTPSN KKKNDLMNRT FKKMMDEYNT KKKKLIKCIK |
| 181 | NHENDFNKIC MDMKNYGTNL FEQLSCYNNN FCNINGIRYH YDEYIHKLIL SVKSKNLNKD |
| 241 | LSDMTNILQQ SELLLTNLNK KMGSYIYIDT IKFIHKEMKH IFNRIEYHTK IINDKTKIIQ |
| 301 | DKIKLNIWRT FQKDELLKRI LDMSNEYSLF ITSDHLRQML YNTFYSKEKH LNNIFHHLIY |
| 361 | VLQMKFNDVP IKMEYFQTYK KNKPLTQ |

SEQ ID NO: 24: PfRH5 amino acid sequence (3D7) excluding signal sequence and flexible
N-terminal region (amino acids 1 to 159)

| | |
|---|---|
| 1 | SIDILQEKEG HLDFVIIPHY TFLDYYKHLS YNSIYHKSST YGKCIAVDAF IKKINETYDK |
| 61 | VKSKCNDIKN DLIATIKKLE HPYDINNKND DSYRYDISEE IDDKSEETDD ETEEVEDSIQ |
| 121 | DTDSNHTPSN KKKNDLMNRT FKKMMDEYNT KKKKLIKCIK NHENDFNKIC MDMKNYGTNL |
| 181 | FEQLSCYNNN FCNTNGIRYH YDEYIHKLIL SVKSKNLNKD LSDMTNILQQ SELLLTNLNK |
| 241 | KMGSYIYIDT IKFIHKEMKH IFNRIEYHTK IINDKTKIIQ DKIKLNIWRT FQKDELLKRI |
| 301 | LDMSNEYSLF ITSDHLRQML YNTFYSKEKH LNNIFHHLIY VLQMKFNDVP IKMEYFQTYK |
| 361 | KNKPLTQ |

SEQ ID NO: 25: PfRH5 amino acid sequence (7G8) excluding signal sequence and flexible
N-terminal region (amino acids 1 to 159)

| | |
|---|---|
| 1 | SIDILQEKEG HLDFVIIPHY TFLDYYKHLS YNSIYHKSST YGKYIAVDAF IKKINETYDK |
| 61 | VKSKCNDIKN DLIATIKKLE HPYDINNKND DSYRYDISEE IDDKSEETDD ETEEVEDSIQ |
| 121 | DTDSNHTPSN KKKNDLMNRT FKKMMDEYNT KKKKLIKCIK NHENDFNKIC MDMKNYGTNL |
| 181 | FEQLSCYNNN FCNTNGIRYH YDEYIHKLIL SVKSKNLNKD LSDMTNILQQ SELLLTNLNK |
| 241 | KMGSYIYIDT IKFIHKEMKH IFNRIEYHTK IINDKTKIIQ DKIKLNIWRT FQKDELLKRI |
| 301 | LDMSNEYSLF ITSDHLRQML YNTFYSKEKH LNNIFHHLIY VLQMKFNDVP IKMEYFQTYK |
| 361 | KNKPLTQ |

SEQ ID NO: 26: PfRH5 amino acid sequence (3D7) excluding signal sequence, flexible N-
terminal (amino acids 1 to 139) and flexible loop (amino acids 248 to 296) regions (3D7
RH5ΔNL)

| | |
|---|---|
| 1 | KNVNFLQYHF KELSNYNIAN SIDILQEKEG HLDFVIIPHY TFLDYYKHLS YNSIYHKSST |
| 61 | YGKCIAVDAF IKKINETYDK VKSKCNDIKN DLIATIKKLE HPYDINNKNR TFKKMMDEYN |
| 121 | TKKKKLIKCI KNHENDFNKI CMDMKNYGTN LFEQLSCYNN NFCNINGIRY HYDEYIHKLI |
| 181 | LSVKSKNLNK DLSDMTNILQ QSELLLTNLN KKMGSYIYID TIKFIHKEMK HIFNRIEYHT |
| 241 | KIINDKTKII QDKIKLNIWR TFQKDELLKR ILDMSNEYSL FITSDHLRQM LYNTFYSKEK |
| 301 | HLNNIFHHLI YVLQMKFNDV PIKMEYFQTY KKNKPLTQ |

SEQ ID NO: 27: PfRH5 amino acid sequence (7G8) excluding signal sequence, flexible N-
terminal (amino acids 1 to 139) and flexible loop (amino acids 248 to 296) regions: (7G8
RH5ΔNL)

| | |
|---|---|
| 1 | KNVNFLQYHF KELSNYNIAN SIDILQEKEG HLDFVIIPHY TFLDYYKHLS YNSIYHKSST |
| 61 | YGKYIAVDAF IKKINETYDK VKSKCNDIKN DLIATIKKLE HPYDINNKNR TFKKMMDEYN |
| 121 | TKKKKLIKCI KNHENDFNKI CMDMKNYGTN LFEQLSCYNN NFCNINGIRY HYDEYIHKLI |
| 181 | LSVKSKNLNK DLSDMTNILQ QSELLLTNLN KKMGSYIYID TIKFIHKEMK HIFNRIEYHT |
| 241 | KIINDKTKII QDKIKLNIWR TFQKDELLKR ILDMSNEYSL FITSDHLRQM LYNTFYSKEK |
| 301 | HLNNIFHHLI YVLQMKFNDV PIKMEYFQTY KKNKPLTQ |

SEQ ID NO: 28: PfRH5 amino acid sequence (3D7) excluding signal sequence, flexible N-
terminal (amino acids 1 to 159) and flexible loop (amino acids 248 to 296) regions

| | |
|---|---|
| 1 | SIDILQEKEG HLDFVIIPHY TFLDYYKHLS YNSIYHKSST YGKCIAVDAF IKKINETYDK |
| 61 | VKSKCNDIKN DLIATIKKLE HPYDINNKNR TFKKMMDEYN TKKKKLIKCI KNHENDFNKI |
| 121 | CMDMKNYGTN LFEQLSCYNN NFCNINGIRY HYDEYIHKLI LSVKSKNLNK DLSDMTNILQ |
| 181 | QSELLLTNLN KKMGSYIYID TIKFIHKEMK HIFNRIEYHT KIINDKTKII QDKIKLNIWR |
| 241 | TFQKDELLKR ILDMSNEYSL FITSDHLRQM LYNTFYSKEK HLNNIFHHLI YVLQMKFNDV |
| 301 | PIKMEYFQTY KKNKPLTQ |

SEQ ID NO: 29: PfRH5 amino acid sequence (7G8) excluding signal sequence, flexible N-
terminal (amino acids 1 to 159) and flexible loop (amino acids 248 to 296) regions

| | |
|---|---|
| 1 | SIDILQEKEG HLDFVIIPHY TFLDYYKHLS YNSIYHKSST YGKYIAVDAF IKKINETYDK |
| 61 | VKSKCNDIKN DLIATIKKLE HPYDINNKNR TFKKMMDEYN TKKKKLIKCI KNHENDFNKI |
| 121 | CMDMKNYGTN LFEQLSCYNN NFCNINGIRY HYDEYIHKLI LSVKSKNLNK DLSDMTNILQ |
| 181 | QSELLLTNLN KKMGSYIYID TIKFIHKEMK HIFNRIEYHT KIINDKTKII QDKIKLNIWR |
| 241 | TFQKDELLKR ILDMSNEYSL FITSDHLRQM LYNTFYSKEK HLNNIFHHLI YVLQMKFNDV |
| 301 | PIKMEYFQTY KKNKPLTQ |

SEQ ID NO: 30: RH5.1
ENAIKKTKNQENNLALLPIKSTEEEKDDIKNGKDIKKEIDNDKENIKTNNAKDHSTYIKSYLNTNVNDGLKYLFIPSHN
SFIKKYSVFNQINDGMLLNEKNDVKNNEDYKNVDYKNVNFLQYHFKELSNYNIANSIDILQEKEGHLDFVIIPHYTFL
DYYKHLSYNSIYHKSSTYGKCIAVDAFIKKINEAYDKVKSKCNDIKNDLIATIKKLEHPYDINNKNDDSYRYDISEEIDDK
SEETDDETEEVEDSIQDTDSNHAPSNKKKNDLMNRAFKKMMDEYNTKKKKLIKCIKNHENDFNKICMDMKNYGT
NLFEQLSCYNNNFCNTNGIRYHYDEYIHKLILSVKSKNLNKDLSDMTNILQQSELLLTNLNKKMGSYIYIDTIKFIHKE
MKHIFNRIEYHTKIINDKTKIIQDKIKLNIWRTFQKDELLKRILDMSNEYSLFITSDHLRQMLYNTFYSKEKHLNNIFHH
LIYVLQMKFNDVPIKMEYFQTYKKNKPLTQ
SEQ ID NO: 31: Full length RH5.1, Bold = BiP Secretion Signal Peptide for Expression
in Insect Cells, Underlined = C-Tag for affinity purification
MKLCILLAVVAFVGLSLGENAIKKTKNQENNLALLPIKSTEEEKDDIKNGKDIKKEIDNDKENIKTNNAKDHSTYIKSY
LNTNVNDGLKYLFIPSHNSFIKKYSVFNQINDGMLLNEKNDVKNNEDYKNVDYKNVNFLQYHFKELSNYNIANSIDI
LQEKEGHLDFVIIPHYTFLDYYKHLSYNSIYHKSSTYGKCIAVDAFIKKINEAYDKVKSKCNDIKNDLIATIKKLEHPYDIN
NKNDDSYRYDISEEIDDKSEETDDETEEVEDSIQDTDSNHAPSNKKKNDLMNRAFKKMMDEYNTKKKKLIKCIKNH
ENDFNKICMDMKNYGTNLFEQLSCYNNNFCNTNGIRYHYDEYIHKLILSVKSKNLNKDLSDMTNILQQSELLLTNLN
KKMGSYIYIDTIKFIHKEMKHIFNRIEYHTKIINDKTKIIQDKIKLNIWRTFQKDELLKRILDMSNEYSLFITSDHLRQML
YNTFYSKEKHLNNIFHHLIYVLQMKFNDVPIKMEYFQTYKKNKPLTQEPEA
SEQ ID NO: 32: PfRH5 (PfRH5FL) sequence of amino acids E26-Q526 of the 3D7 clone
P. falciparum reference sequence, with mutations to delete N-linked glycosylation (N38Q
and N214Q) and with an additional C-terminal AviTagTM and Strep-II ® tag in tandem

| | |
|---|---|
| ENAIKKTKNQ ENQLTLLPIK STEEEKDDIK NGKDIKKEID NDKENIKTNN AKDHSTYIKS | 60 |
| YLNTNVNDGL KYLFIPSHNS FIKKYSVFNQ INDGMLLNEK NDVKNNEDYK NVDYKNVNFL | 120 |
| QYHFKELSNY NIANSIDILQ EKEGHLDFVI IPHYTFLDYY KHLSYNSIYH KSSTYGKCIA | 180 |
| VDAFIKKIQE TYDKVKSKCN DIKNDLIATI KKLEHPYDIN NKNDDSYRYD ISEEIDDKSE | 240 |
| ETDDETEEVE DSIQDTDSNH TPSNKKKNDL MNRTFKKMMD EYNTKKKKLI KCIKNHENDF | 300 |
| NKICMDMKNY GTNLFEQLSC YNNNFCNING IRYHYDEYIH KLILSVKSKN LNKDLSDMTN | 360 |
| ILQQSELLLT NLNKKMGSYI YIDTIKFIHK EMKHIFNRIE YHTKIINDKT KIIQDKIKLN | 420 |
| IWRTFQKDEL LKRILDMSNE YSLFITSDHL RQMLYNTFYS KEKHLNNIFH HLIYVLQMKF | 480 |
| NDVPIKMEYF QTYKKNKPLT QGSASGLNDI FEAQKIEWHE WSHPQFEK | 528 |

SEQ ID NO: 33: PfRH5ΔNL

| | |
|---|---|
| KNVNFLQYHF KELSNYNIAN SIDILQEKEG HLDFVIIPHY TFLDYYKHLS YNSIYHKSST | 60 |
| YGKCIAVDAF IKKINEAYDK VKSKCNDIKN DLIATIKKLE HPYDINNKNR AFKKMMDEYN | 120 |
| TKKKKLIKCI KNHENDFNKI CMDMKNYGTN LFEQLSCYNN NFCNINGIRY HYDEYIHKLI | 180 |
| LSVKSKNLNK DLSDMTNILQ QSELLLTNLN KKMGSYIYID TIKFIHKEMK HIFNRIEYHT | 240 |
| KIINDKTKII QDKIKLNIWR TFQKDELLKR ILDMSNEYSL FITSDHLRQM LYNTFYSKEK | 300 |
| HLNNIFHHLI YVLQMKFNDV PIKMEYFQTY KKNKPLTQEP EA | 342 |

### EXAMPLES

### Example 1: Coformulation of RH5.1 Vaccines with RIPR and CyRPA does not Improve Parasite Growth Inhibitory Activity (GIA)

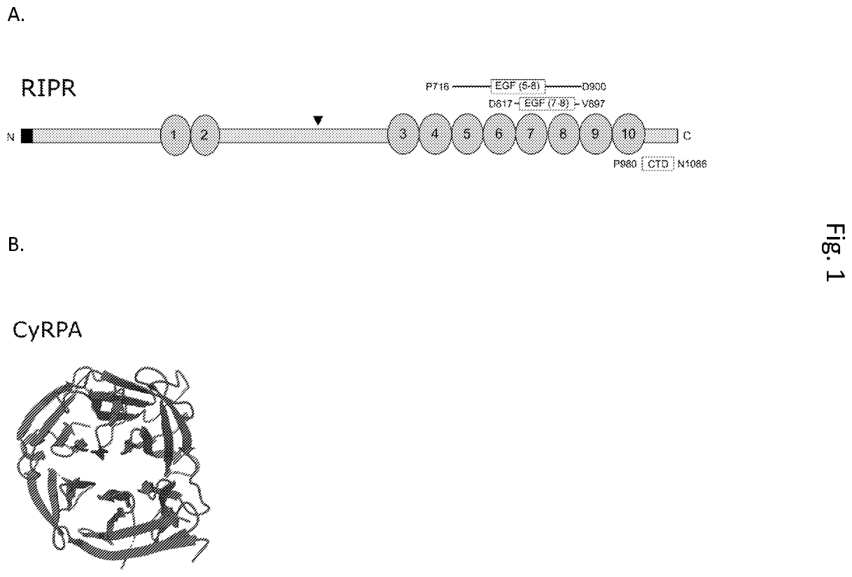

In recent years two essential parasite invasion proteins have been identified. These proteins are known as CyRPA and RIPR. The proteins form a heterotrimeric complex with RH5 that is known as the RH5-CyRPA-RIPR (RCR) complex (FIGS. 1A and B).

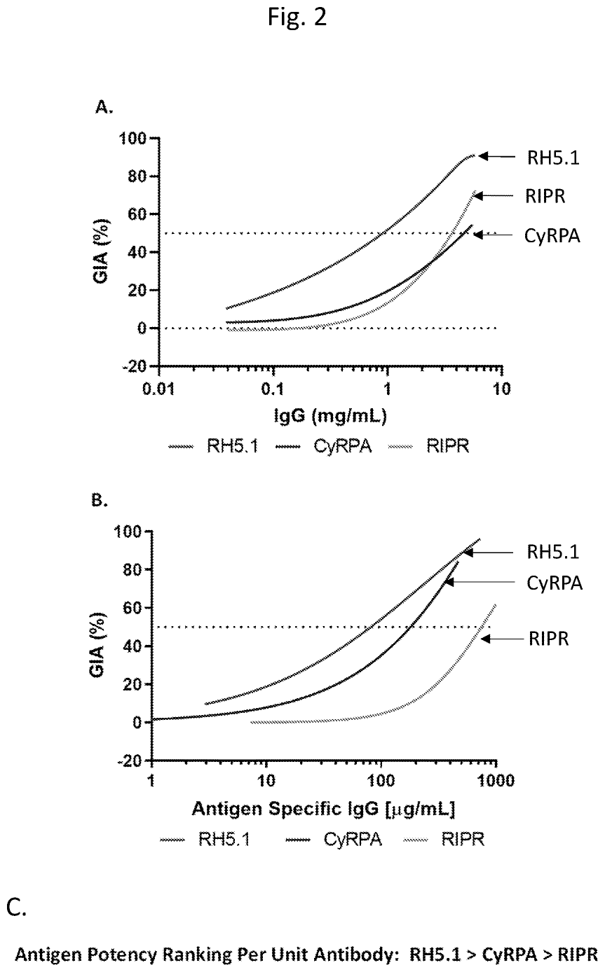

Vaccination of rats with recombinant RH5, CyRPA or RIPR protein has shown that RH5 induces the most potent growth inhibitory polyclonal IgG antibodies, while RIPR induces the least potent growth inhibitory polyclonal IgG antibodies (FIG. 2).

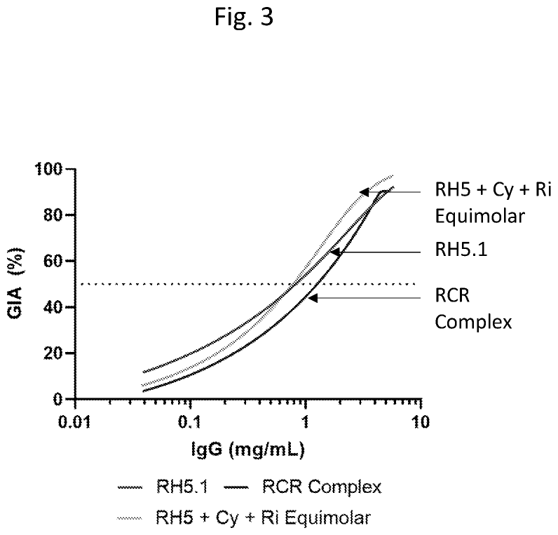

Attempts to improve RH5.1 vaccines by co-formulation with RIPR and CyRPA (either as a mixture or RCR complex) have resulted in no improvement in the parasite growth inhibitory activity (GIA) (FIG. 3).

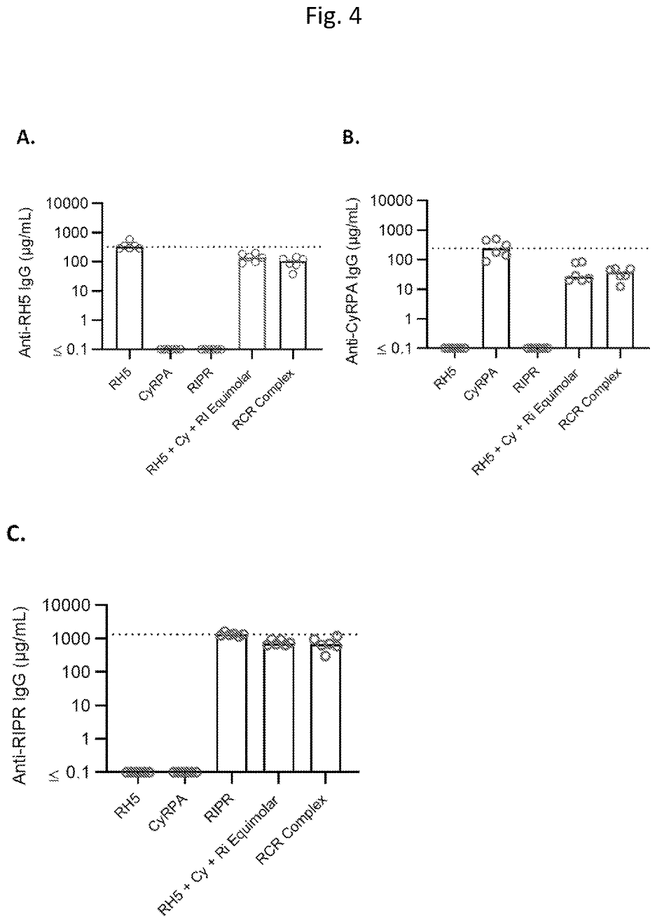

The inventors identified that the lack of improvement in GIA is due to the immunodominance of RIPR, and the subsequent reduction in antibody response to CyRPA and RH5 (FIG. 4).

### Example 2: Antibodies Raised Following Immunisation with Soluble RIPR EGF Domains 5 to 8 Demonstrate Better GIA than Full Length RIPR

To reduce the presence of immunodominant non-inhibitory RIPR antibodies, depletion assays were run using protein fragments of RIPR. These assays identified that all growth inhibitory antibodies raised to full-length RIPR bind to RIPR EGF domains 5-8. In support of this, antibodies raised following immunisation with soluble RIPR EGF domains 5-8 demonstrate better GIA than full length RIPR (FIG. 5).

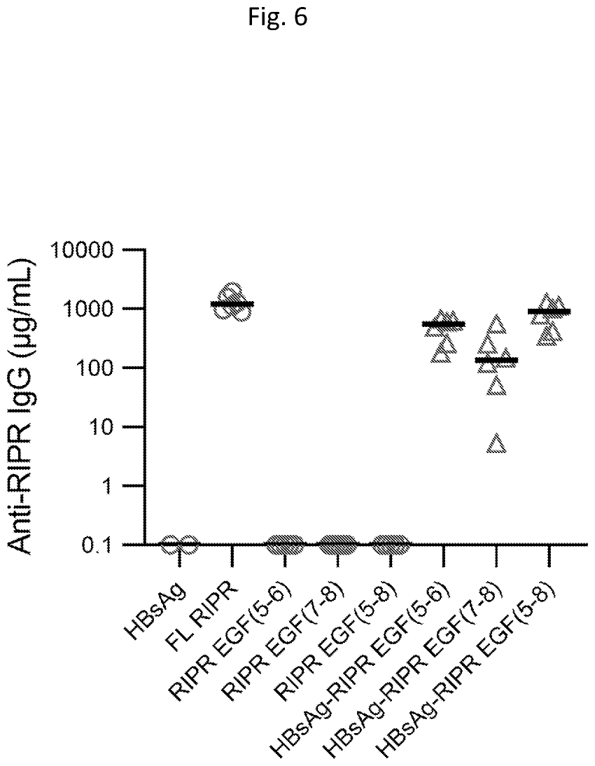

However, vaccination with purified RIPR fragments targeting a single EGF domain or multiple EGF domains from this region are often poorly immunogenic when administered alone or at low doses, unless conjugated to a virus-like particle (VLP) (FIG. 6).

### Example 3: Fusions Comprising a RIPR Fragment and CyRPA have Improved Immunogenicity

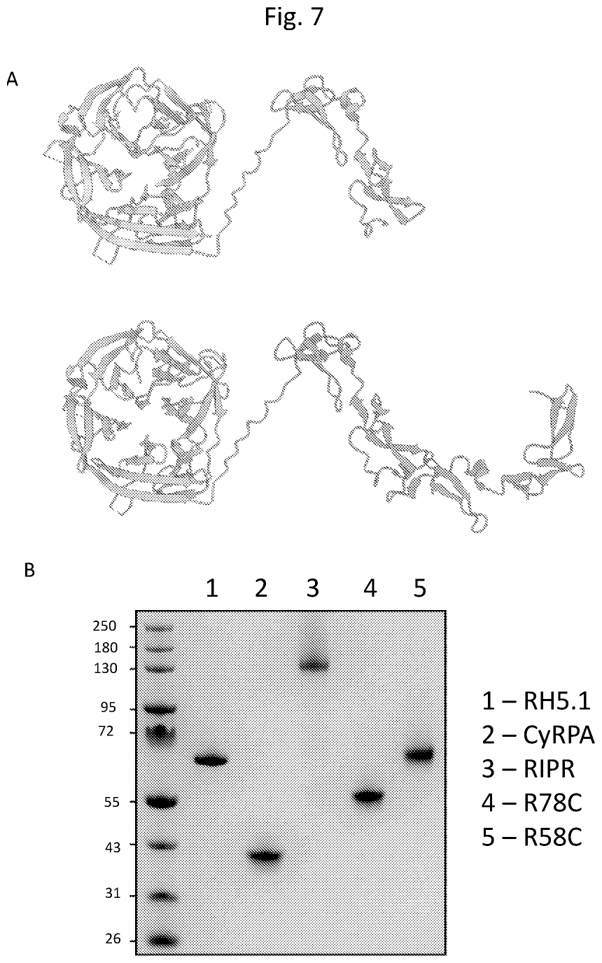

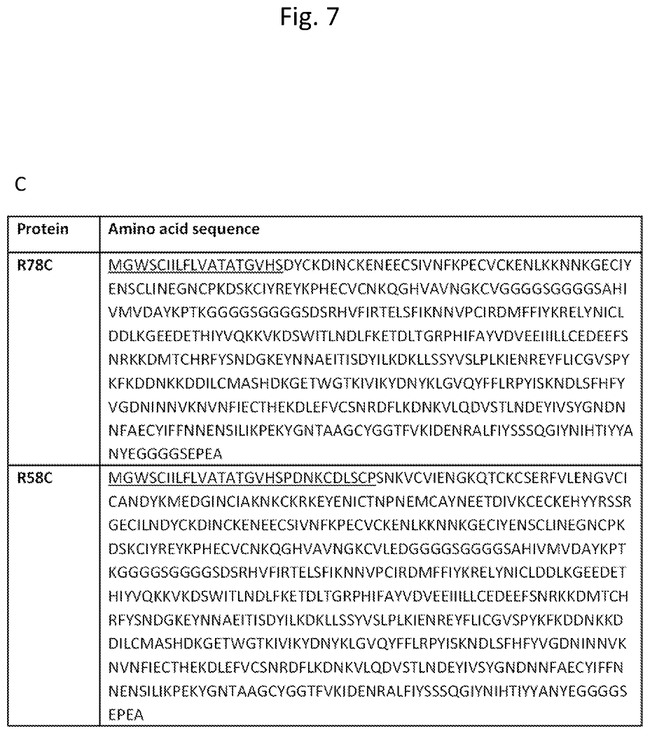

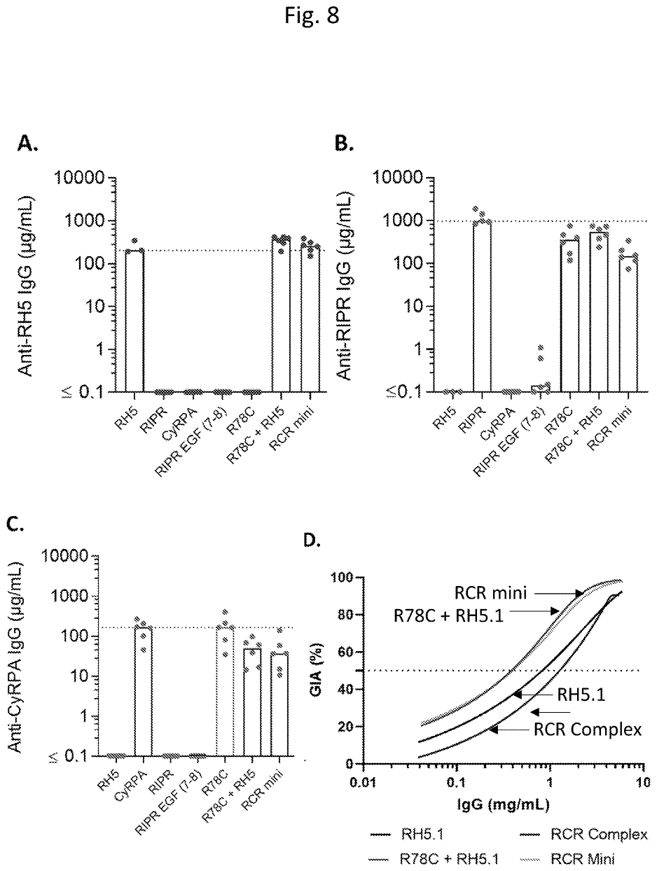

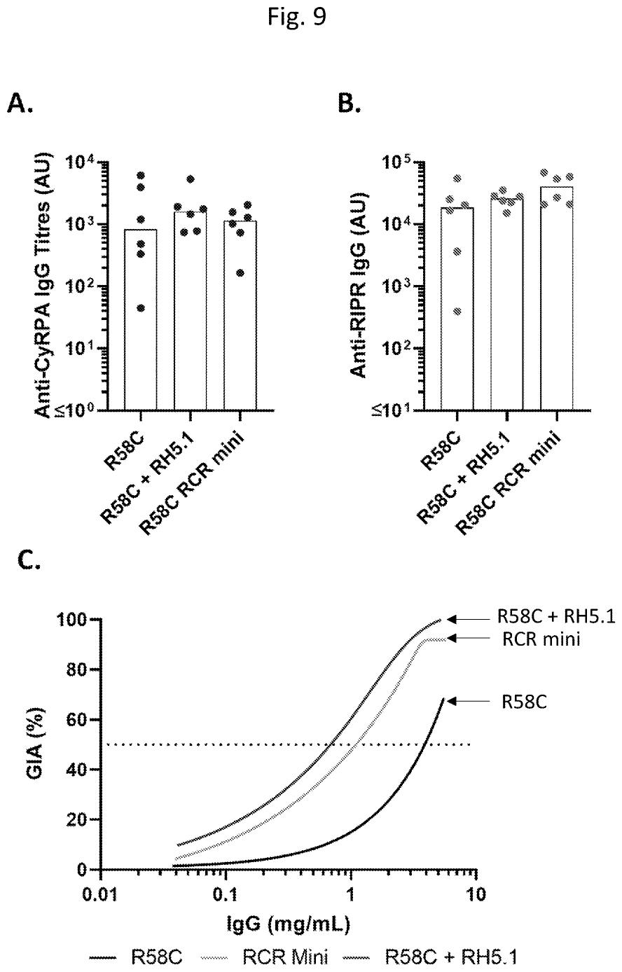

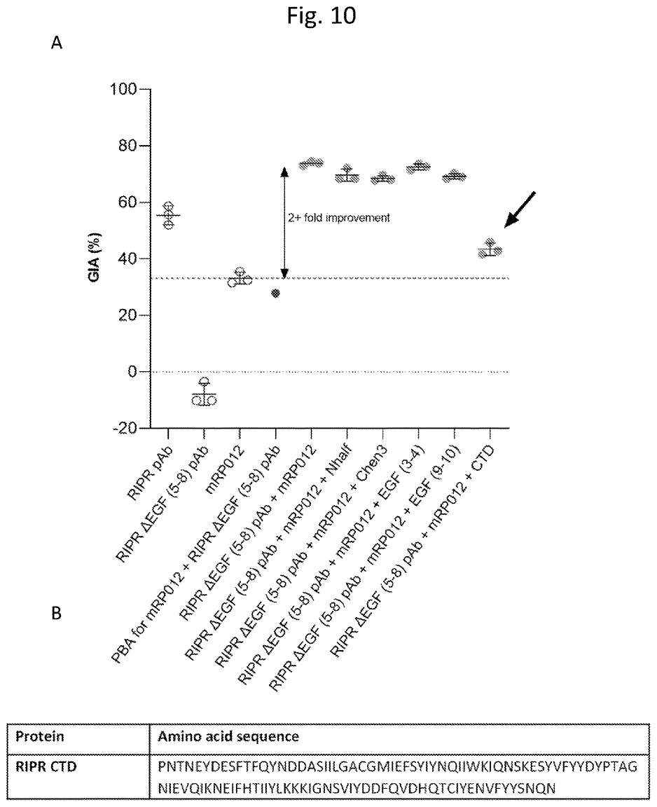

To improve the immunogenicity of the RIPR antigens, RIPR fragments comprising either EGF domains 5 to 8 or EGF domains 7 to 8 were fused to CyRPA. The RIPR antigens and CyRPA antigens were connected using a peptide linker. Specifically, the linker was designed to include a 13 amino acid SpyTag enabling future conjugation to SpyCatcher-based vaccine platforms and facilitating dual antigen display (FIG. 7).

Of the proteins, the fusion comprising RIPR EGF domains 7 and 8 and CyRPA demonstrates enhanced antibody responses when compared to an antigen comprising RIPR EGF domains 7 and 8 alone (FIG. 8B,C). In addition, upon co-formulation of with RH5.1 (as a mixture or complex), the fusion comprising RIPR EGF domains 7 and 8 and CyRPA reduced immune interference (FIG. 8A-C). Surprisingly, a three-fold improvement in the GIA of the purified IgG with respect to RH5.1 alone was also demonstrated.

Similarly, the fusion of RIPR EGF domains 5-8 and CyRPA was shown to be immunogenic and exhibit GIA alone and in combination with RH5.1 (FIG. 9).

Thus, the present inventors have shown that CyRPA and RIPR EGF domain fusion proteins can be used as a novel combination vaccine (FIG. 8D).

### Example 4: Fusion of the CTD to R78C or R58C (to Make R78CC or R58CC) is Likely to Improve the Potency of the Vaccines

The C-terminal domain (CTD) of RIPR was previously shown to have no neutralising activity (FIG. 5B). However using RIPR EGF (5-8) depleted anti-RIPR full-length polyclonal IgG (RIPR delta EGF (5-8) pAb) that has no neutralising GIA activity, the inventors have surprisingly demonstrated that the non-neutralising IgG potentiates the activity of an anti-RIPR monoclonal antibody (mRP012) (FIG. 10A). The sequence of the RIPR CTD is shown in (FIG. 10B). The potentiating effect was due to the action of antibodies against the CTD of RIPR. A variety of protein fragments were tested to determine whether they could reverse the potentiating GIA (Nhalf, Chen3, EGF (3-4), EGF (9-10) and CTD). Only CTD reversed the GIA (arrow). These data suggest that fusion of the CTD to R78C or R58C (to make R78CC or R58CC) has the potential to improve the potency of the vaccines of the invention.

1. A vaccine composition comprising a RH5-Interacting Protein (RIPR) antigen, wherein said antigen is a RIPR fragment comprising one or more RIPR epidermal-growth factor like (EGF) domains.

2. The composition according to claim 1, comprising a contiguous sequence of no more than 400 amino acids from the C-terminal portion of RIPR; optionally comprising no more than 300 amino acids, no more than 200 amino acids or no more than 100 amino acids from the C-terminal portion of RIPR.

3. The composition according to claim 1 or 2, wherein the RIPR antigen comprises (or consists of):
(a) an amino acid sequence having at least 80% sequence identity to SEQ ID NO: 2;
(b) EGF domains 5-8 of RIPR (e.g., SEQ ID NO: 1); and/or
(c) SEQ ID NO: 2.

4. The composition according to any one of the preceding claims, wherein the RIPR antigen comprises (or consists of):
(a) an amino acid sequence having at least 80% sequence identity to SEQ ID NO: 3;
(b) EGF domains 7-8 or RIPR (e.g., SEQ ID NO: 1); and/or
(c) SEQ ID NO: 3.

5. The composition according to any one of the preceding claims, wherein the RIPR antigen lacks:
(i) at least 500 amino acids; preferably 600 amino acids; more preferably 700 contiguous amino acids from SEQ ID NO: 1;
(ii) EGF domains 1-4 of RIPR (e.g., SEQ ID NO: 1); and/or
(iii) EGF domains 9-10 of RIPR (e.g., SEQ ID NO: 1).

6. The composition according to any one of the preceding claims, wherein:
(a) the RIPR antigen comprises a RIPR C-terminal domain (CTD) or fragment thereof, or
(b) the RIPR antigen is fused to a RIPR C-terminal domain (CTD) or fragment thereof.

7. The composition according to claim 6, wherein the CTD comprises (or consists of):
(a) at least 80 amino acids, at least 90 amino acid or at least 100 amino acid from SEQ ID NO: 4;
(b) a sequence having at least 80% sequence identity to SEQ ID NO: 4; and/or
(c) SEQ ID NO: 4.

8. The composition according to any one of the preceding claims, wherein the RIPR antigen comprises (or consists of):
(a) a sequence having at least 80% sequence identity to SEQ ID NO: 5, or SEQ ID NO: 6; preferably SEQ ID NO: 6; or
(b) SEQ ID NO: 5 or SEQ ID NO: 6; preferably SEQ ID NO: 6.

9. The composition according to any one of the preceding claims, wherein the RIPR antigen induces:
(a) antibodies that have reduced immunodominance compared to antibodies raised against full-length RIPR;
(b) a higher proportion of inhibitory antibodies compared to full-length RIPR; and/or
(c) antibodies that have increased growth inhibitory activity (GIA) against the blood-stage Plasmodium parasite compared to antibodies raised against full-length RIPR.

10. The composition according to any one of the preceding claims, wherein the RIPR antigen induces antibodies that have increased growth inhibitory activity (GIA) of at least 50% against the blood-stage Plasmodium parasite; wherein optionally:
(a) the growth inhibitory activity (GIA) of at least 50% is against a plurality of genetic strains of the blood-stage Plasmodium parasite; and/or
(b) the Plasmodium parasite is Plasmodium falciparum.

11. A vaccine composition comprising a fusion protein, the fusion protein comprising the RH5-interacting Protein (RIPR) antigen as defined in any one of the preceding claims and a CyRPA antigen.

12. The composition of claim 11, wherein the CyRPA antigen:
(a) comprises (or consists) of at least 200 amino acids, at least 250 amino acids, or at least 300 amino acids from SEQ ID NO: 7
(b) comprises (or consists) of a sequence having at least 80% sequence identity to SEQ ID NO: 7;
(c) is N-terminal to or C-terminal to the RIPR antigen; and/or
(d) is (i) fused directly to the RIPR antigen; or (ii) attached to the RIPR antigen by a linker; optionally
wherein the linker:
i. is a peptide linker;
ii. is capable of ligating to another polypeptide; and/or
iii. comprises (or consists of) SEQ ID NO: 8 or SEQ ID NO: 9.

13. The composition of claim 11 or 12, wherein the sequence of the fusion protein comprises (or consists of):
(a) a sequence having at least 80% sequence identity to any one of SEQ ID NO: 10 to SEQ ID NO: 13, SEQ ID NO: 15, SEQ ID NO: 16, SEQ ID NO: 18 and SEQ ID NO: 19; or
(b) any one of SEQ ID NO: 10 to SEQ ID NO: 13, SEQ ID NO: 15, SEQ ID NO: 16, SEQ ID NO: 18 and SEQ ID NO: 19.

14. A vaccine composition comprising a virus-like particle (VLP), wherein the VLP comprises the RH5-interacting Protein (RIPR) antigen as defined in any one of claims 1 to 10 or the fusion protein as defined in any one of claims 11 to 13.

15. The vaccine composition of any one of the preceding claims further comprising one or more additional Plasmodium merozoite antigen, wherein optionally:
(a) the one or more additional Plasmodium merozoite antigen is Reticulocyte-binding protein Homologue 5 (PfRH5) antigen;
(b) the one or more additional Plasmodium merozoite antigen is expressed as a fusion protein;
(c) the one or more additional Plasmodium merozoite antigen is comprised in a VLP; and/or
(d) the one or more additional Plasmodium merozoite antigen is expressed as a single polypeptide with the RH5-interacting Protein (RIPR) antigen as defined in any one of claims 1 to 10 or the fusion protein as defined in any one of claims 11 to 13.

16. A viral vector, RNA vaccine or DNA plasmid comprising a polynucleotide sequence encoding a RH5-interacting Protein (RIPR) antigen as defined in any one of claims 1 to 10 or the fusion protein as defined in any one of claims 11 to 13.

17. The viral vector of claim 16, wherein the viral vector is a human or simian adenovirus, or a pox virus, optionally wherein the viral vector is an AdHu5, ChAd63, ChAdOX1, ChAdOX2 or modified vaccinia Ankara (MVA) vector.

18. The RNA vaccine or DNA plasmid of claim 16, wherein the RNA vaccine or DNA plasmid is capable of expression in an immunised mammalian cell.

19. The DNA plasmid of claim 16 or 18, wherein the DNA plasmid is capable of expression in a heterologous protein expression system.

20. The viral vector, RNA vaccine or DNA plasmid of any one of claims 16 to 19, wherein:
(a) the viral vector, RNA vaccine or DNA plasmid further expresses a Reticulocyte-binding protein Homologue 5 (PfRH5) antigen; optionally wherein (i) the PfRH5 antigen has at least 90% sequence identity to any one of SEQ ID NO: 20 to 33; and/or (ii) the RIPR antigen and the PfRH5 antigen are expressed as a fusion protein; and/or
(b) the viral vector, RNA vaccine or DNA plasmid further expresses one or more antigens selected from PfAMA1, PfCSS, PHPTRAMP, PfEBA175, PfRH1, PfRH2a, PfRH2b, PfRH4 and/or PfAARP, or a fragment thereof.

21. The viral vector, RNA vaccine or DNA plasmid of any one of claims 16 to 20, in combination with:
(a) (i) a viral vector, RNA vaccine or DNA plasmid that expresses a Reticulocyte-binding protein Homologue 5 (PfRH5) antigen; (ii) a PfRH5 antigen or a fusion protein comprising a PfRH5 antigen; or (iii) a VLP comprising a PfRH5 antigen or PfRH5 fusion protein; and/or
(b) (i) a viral vector, RNA vaccine or DNA plasmid that expresses one or more Plasmodium merozoite antigens selected from PfRH5, PfAMA1, PfCSS, PfPTRAMP, PfEBA175, PfRH1, PfRH2a, PfRH2b, PfRH4 and/or PfAARP, or a fragment thereof; (ii) said Plasmodium merozoite antigen or a fusion protein comprising said Plasmodium merozoite antigen; or (iii) a VLP comprising a Plasmodium merozoite antigen or Plasmodium merozoite antigen fusion protein.

22. An antibody, or binding fragment thereof, that specifically binds to a RIPR antigen as defined in any one of claims 1 to 10, a fusion protein as defined in any one of claims 11 to 13 or a VLP as defined in claim 14, optionally wherein the antibody is:
(a) a monoclonal or polyclonal antibody; and/or
(b) Fab, F(ab′)2, Fv, scFv, Fd or dAb.

23. A vaccine composition comprising:
